(19) United States
(12) Patent Application Publication
(10) Pub. No.: US 2008/0050429 A1
(43) Pub. Date: Feb. 28, 2008

(54) Low flush niacin formulation

(76) Inventors: Jose G. Rocca, Miami, FL (US); Eugenio Cefali, Fort Lauderdale, FL (US); Yucun Zhu, Weston, FL (US)

Correspondence Address: KOS PHARMACEUTICALS, INC., 1 Cedar Brook Drive, Cranbury, NJ, 08512, US

(21) Appl. No.: 11/705,675

(22) Filed: Feb. 13, 2007

Related U.S. Application Data
(60) Provisional application No. 60/774,339, filed on Feb. 17, 2006.

(51) Int. Cl. A61K 31/455; A61K 38/16; A61K 9/14; A61K 9/22; A61P 7/00; A61P 9/08

(52) U.S. Cl. 424/465; 424/489; 514/12; 514/356

(57) ABSTRACT

The invention relates to an extended-release matrix formulation capable of being directly compressed into tablets comprising niacin, a release-retarding agent, and other excipients. The resulting tablets of the invention demonstrate favorable release characteristics and a reduction in the severity, duration and incidences of cutaneous flushing commonly associated with niacin treatment.

### FIELD OF THE INVENTION

The invention relates to an extended-release matrix formulation capable of being directly compressed into tablets comprising niacin, a release-retarding agent, and other excipients. The resulting tablets of the invention demonstrate improved manufacturing characteristics, favorable release characteristics and a reduction in the duration, severity and the incidence of cutaneous flushing commonly associated with niacin treatment.

### BACKGROUND OF THE INVENTION

Niacin (nicotinic acid, also known as 3-pyridinecarboxylic acid, chemical formula C₆H₅NO₂) is known to have benefits associated with the treatment of hypercholesterolemia because it increases levels of high-density lipoproteins (HDL) and lowers levels of total serum cholesterol low-density lipoproteins (LDL) and triglycerides.

Although niacin is known to provide a very beneficial effect on blood lipids, with the exception of NIASPAN® (Kos Pharmaceuticals, Inc., Cranbury, N.J.), widespread use of niacin is limited due to the high incidence of “flush” that often occurs with the higher doses of niacin needed for effective lipid treatment. Flushing is a term generally used to describe niacin-induced vasodilatation. As a result, an individual experiencing flushing may develop a visible, uncomfortable hot or flushed feeling upon administration of niacin. While certain materials and/or formulations have been suggested for avoiding or reducing cutaneous flushing (see U.S. Pat. Nos. 4,956,252, 5,023,245 and 5,126,145), this unwanted side-affect remains a problem for wide scale utilization of niacin products.

Further, the current release retarding agent (also commonly referred to as a “swelling agent”) in the commercial NIASPAN® formulations is highly variable in quality, thereby leading to the need for special batch production from a commercial supplier to met internal specifications.

Therefore, there is a need in the pharmaceutical arts for an extended-release nicotinic acid formulation that provides reduced levels of cutaneous flushing over existing niacin formulations, while also allowing for a robust manufacturing process characterized by improved physical, chemical and mechanical properties.

### SUMMARY OF THE INVENTION

The present invention provides for an extended-release (ER) tablet formulation comprising niacin and a release-retarding agent. In one embodiment, the invention provides a 1000 mg ER niacin tablet formulation with improved flowability, compressibility, compactability and hardness than existing 1000 mg prescription niacin formulations. In addition, the 1000 mg ER niacin tablets of the current invention demonstrate an ability to duplicate the release rate and/or absorption rate of commercially available 500 mg NIASPAN® tablets, without any reduction in manufacturing robustness (a robust process is one that has the ability to reproduce a target endpoint under varying circumstances or conditions, such as small changes in raw materials or manufacturing processes) or commercial desirability (e.g., size). Because two 500 mg NIASPAN® tablets are believed to be characterized by less flushing than one 1000 mg NIASPAN® tablet, one object of the invention is to provide a 1000 mg ER niacin tablet formulation that is bioequivalent to two 500 mg NIASPAN® tablets.

In particular, the present invention provides a pharmaceutical composition comprising:

(a) about 70% to about 92% w/w of niacin;

(b) about 7% to about 25% w/w of a release-retarding agent;

(c) about 0.1% to about 4.3% w/w of a binder, and

(d) about 0.5% to about 1.5% w/w of a lubricant.

In one embodiment the pharmaceutical tablet is a direct compression tablet.

Further, the present invention provides methods of preparing the extended-release niacin tablets which comprises the steps of:
(a) blending a mixture of about 70% to about 92% w/w of niacin, about 7% to about 25% w/w of a release-retarding agent, about 0.1% to about 4.3% w/w of a binder, and about 1.3% to about 4.3% w/w of a lubricant; and
(b) compressing the mixture of step (a) into a tablet.

In a preferred embodiment, the extended-release niacin tablet is prepared by blending granular niacin.

Also provided is a method of reducing flushing associated with niacin treatment therapy in a patient, wherein said method comprises administering the extended-release niacin tablets forms of the present invention to a patient in need of niacin treatment. In a preferred embodiment, a niacin formulation according to the present invention is administered once-daily in the evening or at night.

One embodiment of the invention comprises a reformulated 1000 mg extended-release niacin pharmaceutical composition which when administered to subjects in a bioequivalence study comparing a single dose of four 500 mg NIASPAN® tablets to a single dose of to of said reformulated 1000 mg extended-release niacin compositions provides 90% CI's for a natural-log transformed ratio of the appropriate bioavailability parameters within a 80% to 125% interval.

According to the present invention, flushing can be further reduced by administering an extended-release niacin formulation of the present invention in combination with a non-steroidal anti-inflammatory drug (NSAID). In a preferred embodiment, the NSAID is aspirin.

A pharmaceutical composition according to the present invention can include an immediate-release flush-inhibiting agent component and a delayed-release niacin component, wherein the niacin has a delayed-release (i.e., the niacin is released after a lag time). In a preferred embodiment, the niacin is released at least about 30 minutes to about 40 minutes after release of the flush-inhibiting agent.

### BRIEF DESCRIPTION OF THE DRAWINGS

FIG. 1 is a graph showing mean niacin dissolution from 1000 mg niacin extended-release tablets containing various levels of METHOCEL® K-15M Premium.

FIG. 2 is a graph showing the effect of varying the viscosity of METHOCEL® K-15 MP CR on niacin dissolution from 1000 mg niacin extended-release tablets (1240 mg total weight).

FIG. 3 is a graph showing the niacin dissolution profiles from 1000 mg niacin extended-release tablets produced using bulk and 40 mesh PVP K-90.

FIG. 4 is a graph showing the niacin dissolution profiles from 1000 mg niacin extended-release tablets (1240 mg total weight) produced using different mixing steps.

FIG. 5 is a flow diagram showing the direct compression manufacturing process.

FIG. 6 is a flow diagram of the clinical study described in Example 3.

FIG. 7 is a bar graph showing the incidence of flushing following administration of two film coated 1000 mg extended-release niacin formulations of the present invention (Test) and two non-coated 1000 mg NIASPAN® tablets (Reference).

FIG. 8 is a bar graph showing the median intensity of the first flushing event following administration of two film coated 1000 mg extended-release niacin formulations of the present invention (Test) and two non-coated 1000 mg NIASPAN® tablets (Reference).

FIG. 9 is a bar graph showing the median duration of the first flushing event following administration of two film coated 1000 mg extended-release niacin formulations of the present invention (Test) and two non-coated 1000 mg NIASPAN® tablets (Reference).

FIG. 10 is a bar graph showing the incidence of individual flushing symptoms in the first flushing event following administration of two film coated 1000 mg extended-release niacin formulations of the present invention (Test) and two non-coated 1000 mg NIASPAN® tablets (Reference).

FIG. 11 is a graph showing the mean plasma concentration of niacin after administration of two 1000 mg extended-release formulations of the present invention (“Test” or “Reformulated”) and two 1000 mg NIASPAN® tablets (“Ref”).

FIG. 12 is a graph showing the mean plasma concentration of NUA after administration of two 1000 mg extended-release formulations of the present invention (“Test” or “Reformulated”) and two 1000 mg NIASPAN® tablets (“Ref”).

FIG. 13 is a bar graph showing the mean urinary recovery of niacin and its metabolites (as a percent of niacin dose) 96 hours after administration of two 1000 mg extended-release formulations of the present invention (“Test” or “Reformulated”) and two 1000 mg NIASPAN® tablets (“Ref”).

FIG. 14a is a graph showing the linear mean plasma niacin profile for three test extended-release niacin formulations (ERN-1, ERN-2, ERN-3) and a reference extended-release niacin formulation (NSP); 14b is a graph showing the semi-log mean plasma niacin profile for the three test and one reference formulations.

FIG. 15a is a graph showing the linear mean plasma NUA profile for three test extended-release niacin formulations (ERN-1, ERN-2, ERN-3) and a reference extended-release niacin formulation (NSP); 15b is a graph showing the semi-log mean plasma NUA profile for the three test and one reference formulations.

FIG. 16 is a bar graph showing the mean urinary recovery of niacin and its metabolites as a percent of niacin dose for three test extended-release niacin formulations (ERN-1, ERN-2, ERN-3) and a reference extended-release niacin formulation (NSP).

FIG. 17a is a graph showing the linear mean plasma niacin profile for two coated 1000 mg extended-release niacin formulations of the present invention (Test) and two uncoated 1000 mg extended-release niacin formulations of the present invention (Ref); 17b is a graph showing the log-transformed mean plasma niacin profile for the test and reference formulations.

FIG. 18a is a graph showing the linear mean plasma NUA profile for two coated 1000 mg extended-release niacin formulations of the present invention (Test) and two uncoated 1000 mg extended-release niacin formulations of the present invention (Ref); 18b is a graph showing the log-transformed mean plasma NUA profile for the test and reference formulations.

FIG. 19 is a bar graph showing the mean urinary recovery of niacin and its metabolites 96 hours after administration of two coated 1000 mg extended-release niacin formulations of the present invention (Test) and two uncoated 1000 mg extended-release niacin formulations of the present invention (Ref).

FIG. 20 is a flow diagram showing the Example 6 study design.

FIG. 21 is a bar graph showing the incidence of individual flushing symptoms for the first flushing event following administration of two 1000 mg extended-release niacin formulations of the present invention (“NIASPAN® CF”) when: (1) the subjects were pretreated with aspirin (ASA), (2) ASA was administered with the niacin formulation, and (3) the niacin formulation was administered alone.

FIG. 22 is a bar graph illustrating the incidence of flushing events for both Example 3 and Example 8.

FIG. 23 is a bar graph illustrating the intensity of flushing events for both Example 3 and Example 8.

### DETAILED DESCRIPTION

The extended-release matrix tablet formulations of the present invention include (1) niacin as an active ingredient and (2) a hydrophilic polymer matrix for achieving extended-release of the active ingredient, i.e., a release-retarding agent. As used herein, an “extended release” formulation means a formulation that provides effective treatment for dyslipidemia in a patient with once-daily dosing.

Extended-release niacin formulations of the present invention can result in an improved lipid profile in a patient. For example, administration of an extended-release niacin formulation of the present invention to a patient can lower total cholesterol, low density lipoprotein (LDL), triglycerides, and lipoprotein A (Lp(a)), and increase high density lipoprotein (HDL) in the patient's bloodstream. A condition, which requires treatment to lower total cholesterol, LDL, triglycerides, and/or lipoprotein A (Lp(a)); and/or increase in HDL in a patient's bloodstream is herein referred to as a “dyslipidemia.” Accordingly, the present invention encompasses the treatment of dyslipidemias by administering an extended-release niacin formulation of the present invention to a patient in need of such treatment.

Bioequivalence is the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. Typically, it is sufficient to demonstrate that the 90% confidence intervals for Test/Reference treatment ratios of natural log-transformed Cₘₐₓ and AUC or any appropriate substitute for these calculated bioequivalence parameters fall between 80% and 125%, inclusive, to conclude that the two formulations are bioequivalent.

Formulations within the scope of the invention are those that are deemed bioequivalent to formulations of the invention when 90% CI's for test/reference treatment ratios of natural log-transformed bioavailability parameters fall within standard 80% to 125% intervals (See for example, Guidance for Industry: Bioavailability and Bioequivalence Studies for Orally Administered Drug Products-General Considerations, U.S. Department of Health & Human Services, Food and Drug Administration, CDER, March 2003; Guidance for Industry Food-Effect Bioavailability and Fed Bioequivalence Studies, December 2002; the contents of both publications are hereby incorporated by reference). As is known to those skilled in the art, such formulations are compared to reference formulations (such as those described herein or the embodiments of the invention described herein) under the same analytical conditions (e.g., analytical and technical conditions analysis) using relevant bioequivalent parameters wherein the reference formula is used as a control.

### Niacin

Niacin, a water-soluble medicament, is commercially available as fine white crystals, granules, or white crystalline powder. Pharmaceutical compositions of the present invention can be produced using niacin crystals, granules or powder. In a preferred embodiment, the pharmaceutical compositions are produced using granular niacin, which has greater flowability compared to niacin powder. Flowability is a critical processing parameter for tablet manufacturing. The use of granular niacin according to the present invention improves flowability and renders direct compression of niacin tablets feasible at production scale. Any granular niacin particle size is suitable for preparing a niacin tablet according to the present invention. A preferred particle size for niacin granular is NLT 85% (w/w) for sieve fraction such that the granules are in the range of 100-425 μm and NMT 10% (w/w) for dust <100 μm. The flowability of niacin powder can be increased using a dry granulation or wet granulation process.

Niacin will typically be present in the tablets of the present invention at a concentration of about 70% to about 95% w/w, preferably about 76% to about 90% w/w, more preferably about 78% to about 82% w/w. Niacin can be present in the extended-release formulations of the present invention in an amount from about 100 mg to 3000 mg. In certain embodiments, a formulation of the present invention includes about 500 mg, about 750 mg, or about 1000 mg of niacin. Preferred daily dosages of niacin are about 1000 mg, about 1500 mg or about 2000 mg. Thus, for example, a daily dosage of niacin can be provided to a patient by administering two 1000 mg tablets to a patient once-daily.

### The Release-Retarding Agent

Extended-release from a polymer matrix system will typically involve polymer wetting, polymer hydration, gel formation, swelling and polymer dissolution. With respect to soluble drugs, these drugs become wet, dissolve and diffuse out of the gel layer formed by the polymer matrix. Although the mechanisms by which soluble drugs are released in matrix tablets are dependent on many variables, the general principle is that a water-soluble polymer, present throughout the tablet, hydrates on the outer tablet surface to form a gel layer. As water permeates into the tablet, the gel layer increases in thickness and the soluble drug diffuses through the gel layer. During the life of the ingested tablet, the rate of drug release is determined by diffusion of the soluble drug through the gel and by the rate of tablet erosion.

The release-retarding component of the present invention may be any agent known to those skilled in the art demonstrating favorable swelling and gelling properties. Examples of suitable release-retarding agents include, but are not limited to, hydroxypropyl cellulose (HPC), hydroxypropyl methyl cellulose (commonly also referred to as HPMC or hypromellose), methylcellulose (MC), hydroxyethyl cellulose (HEC) and polyvinyl pyrrolidone (PVP), xanthan gum, and methacrylate copolymers with trimethylammonioethylmethacrylate (EUDRAGIT RS®, EUDRAGIT RL®), as well as mixtures of these release-retarding agents. In one embodiment, the release-retarding agent is a hydrophilic, water-soluble polymer, Preferred hydrophilic polymers are medium-viscosity hydroxypropyl methyl cellulose and medium-viscosity polyvinyl alcohol.

The release-retarding agent will typically be present in the tablets of the present invention at a concentration of about 7.0% to about 25.0% w/w (percent weight relative to total weight of the formulation), preferably about 11.0% to about 20.0% w/w, more preferably about 14% to about 18% w/w.

In one embodiment, the release-retarding agent is hydroxypropyl methyl cellulose. HPMC has a polymeric backbone of cellulose, a natural carbohydrate that contains a basic repeating structure of anhydroglucose units. The solubility of (e.g., hydration rate), and strength of the gel layer formed by, HPMC is influenced by the proportion of two chemical substituents, hydroxypropoxyl (sometimes referred to as hydroxypropyl) and methoxyl (sometimes referred to as methyl) substitution, attached to the cellulose backbone (cellulose being a natural carbohydrate that contains a basic repeating structure of anhyroglucose units) of HPMC. The hydroxypropoxyl substitution is relatively hydrophilic in nature and greatly contributes to the rate of hydration, while the methoxyl substitution is relatively hydrophobic in nature. The amount of substituent groups on the anhydroglucose units of cellulose can be designated by the average number of substituents groups attached to a single anhydroglucose ring, a concept commonly known to those skilled in the art as “degree of substitution”. See METHOCEL® Cellulose Ethers Technical Handbook, Dow Chemical Company (Published September 2002, Form No. 192-01062-0902 AMS); and Using METHOCEL® Cellulose Eithers for Controlled Release of Drugs in Hydrophilic Matrix Systems (Published July 2002, Form No. 198-02075-0702 AMS). In one embodiment of the invention, the HPMC release-retarding agent has a methoxyl degree of substitution of about 1.2 to about 2.0 and a hydroxypropoxyl molar substitution of about 0.1 to about 0.3, preferably a methoxyl degree of substitution of about 1.4 to about 1.9 and a hydroxypropoxyl molar substitution of about 0.19 to about 0.24, more preferably a methoxyl degree of substitution of about 1.39 to about 1.41 and a hydroxypropoxyl molar substitution of about 0.20 to about 0.22, more preferably a methoxyl degree of substitution of about 1.4 and a hydroxypropoxyl molar substitution of about 0.21. METHOCEL® K-15M (available from Dow Chemical Company, including specific K-15M sub-brands such as K-15M premium and K-15M premium CR) is a preferred release-retarding agent.

Additionally, hydroxypropyl methyl cellulose polymers are commercially available in different viscosity grades. These include, for example, 4000 and 15000 mPas (1 Centipoise (cps)=1 mPas (Millipascal Second)) viscosity grades of METHOCEL® K, i.e. METHOCEL® K4M and METHOCEL® K K15M, available from the Dow Chemical Co, USA; and 4000, 15,000 and 39000 mPas viscosity grades of Metalose 90 SH, available from Shin Etsu Ltd, Japan. In an embodiment of the invention, HPMC viscosity (measured at a 2% concentration in water at 20° C. e.g., ASTM D2363) is about 11,000 to about 22,000 mPas, preferably about 13,000 to about 18,000 mPas.

To determine the specific characteristics necessary for substitution of suitable polymers other than HPMC, one of skill in the art can vary the degree of substitution of the polymer (e.g., hydroxypropyl cellulose) and identify a substitute that matches the dissolution profile of a formulation utilizing HPMC according to the invention (e.g., a formulation according to Example 1 or 2).

### Excipients

The tablets of the present invention further comprise a binder. The binder may be any conventionally known pharmaceutically acceptable binder, such as polyvinylpyrrolidone (also known as PVP, povidone, polyvidone), hydroxypropyl cellulose, hydroxyethyl cellulose, ethylcellulose, polymethacrylate, waxes and the like. Mixtures of the aforementioned binding agents may also be used. In an embodiment of the invention, the binder comprises about 0.1% to 4.3% w/w of the total weight of the tablet, preferably about 0.2% to 3.25% w/w, more preferably about 2.5% to 3.0% w/w.

In addition, the tablets of the present invention comprise a lubricant. The lubricant can be hydrophobic or hydrophilic and include lubricants commonly known to those in the art, such as, but not limited to talc, magnesium stearate, calcium stearate, stearic acid, hydrogenated vegetable oils and the like. Preferably, the lubricant is stearic acid. Addition of a lubricant to the formulation reduces friction between the die wall and tablet formulation during compression, aids in the flow of powder (i.e., the flow of mixed formulation into the hopper and die), and helps prevent adhesion of tablet material to the processing equipment. In one embodiment, the tablet formulations of the invention comprise about 0.5% to 1.5% w/w of a lubricant, preferably about 0.75% to 1.25% w/w, more preferably, about 0.85% to 1.15% w/w, more preferably, about 0.95% to 1.05% w/w.

### Coatings

The extended-release tablet formulations of the invention may further include a coating, as are known in the field of pharmaceutical solid dosage forms to provide a color coat, enhanced visual characteristics, act as a moisture or odor barrier, protect against deterioration by environmental factors like sunlight, temperature variations, or to taste mask the tablets. Such coatings, as are known to those skilled in the art, may contain a polymer, plasticizer and/or color pigment. Examples include OPADRY® coatings The coating can be applied from solution (e.g., aqueous), solvent or suspension using any known means, such as a fluidized bed coater (e.g., Wurster coating) or pan coating system. In one embodiment of the invention, the coating is a color coating, specifically an OPADRY® coating. In a further embodiment, the color coating is applied to the tablet in an amount from about 1.5 to about 8.0% weight gain, and preferably from about 1.75 to about 5.0% weight gain.

### Equivalency to NIASPAN® 500 mg Tablets

A review of previous clinical studies revealed that two (2) NIASPAN® 1000 mg tablets (tablet weight 1203.6 mg) were not bioequivalent to four (4) NIASPAN® 500 mg tablets, and niacin released faster from the NIASPAN® 1000 mg tablets than from the NIASPAN® 500 mg tablets. Further study demonstrated that niacin ER 1000 mg tablets (tablet weight 1419.0 mg) with double the amount of components in the NIASPAN® 500 mg tablet were also not bioequivalent. In the latter case, niacin dissolution was slower from the 1000 mg tablets than from the NIASPAN® 500 mg tablets in vitro, and niacin was absorbed more slowly from the niacin ER 1000 mg tablets than the reference product (500 mg) in vivo. A further study demonstrated that reformulated niacin ER 1000 mg tablets with the tablet weights of 1300.0 mg and 1280.0 mg were also not bioequivalent to NIASPAN® 500 mg tablets due to their slower release rates.

In order to formulate niacin ER 1000 mg tablets bioequivalent to two NIASPAN® 500 mg tablets, the inventors prepared and tested multiple niacin ER 1000 mg formulations in vitro to predict in vivo release and absorption characteristics. The test niacin ER 1000 mg tablets were further reformulated based on the fact that dissolution decreased with increased polymer (release regarding agent) levels in the tablet (w/w). Accordingly, evaluation included new ingredients (such as different types of polymer), and analysis of alternative manufacturing technology (such as direct compression or roller compaction)

Table 1 illustrates various test formulas for 1000 mg tablets having varying total tablet weight.

TABLE 1

| Component | Weight/Tablet (mg) | | | | | |
|---|---|---|---|---|---|---|
| Niacin | 1000.0 | 1000.0 | 1000.0 | 1000.0 | 1000.0 | 1000.0 |
| Granular, USP | | | | | | |
| Release- | 153.5 | 173.3 | 193.1 | 212.9 | 232.7 | 252.5 |
| retarding agent | | | | | | |
| Povidone, USP | 34.5 | 34.5 | 34.5 | 34.5 | 34.5 | 34.5 |
| Stearic Acid | 12.0 | 12.2 | 12.4 | 12.6 | 12.8 | 13.0 |
| NF | | | | | | |
| Formulation | 1200.0 | 1220.0 | 1240.0 | 1260.0 | 1280.0 | 1300.0 |
| Tablet Weight | | | | | | |
| (mg) | | | | | | |

After primary evaluation of multiple variables, the four formulations described below were selected for further evaluation. Variations to the formulations below were analyzed based on dissolution profile using 500 mg NIASPAN® as a reference and employing a USP Type 3 Apparatus in 250 ml of simulated gastric fluid maintained at a pH of 1.2, 37° C. for 60 minutes followed by 250 ml simulated intestinal fluid maintained at a pH of 6.8, 37° C., for all time points.

### (i) METHOCEL® E10M prepared using wet granulation (WG)

Niacin granular, METHOCEL® E10M, Povidone K90, and stearic acid were weighed according to the formulas designated for 1240 mg, 1260 mg, 1280 mg and 1300 mg formulations, and then granulated in a high shear granulator utilizing deionized water as the granulating solution. The wet granules were dried, milled, and then blended with extragranular METHOCEL® E10M and stearic acid. The final well-blended mixture was compressed into tablets using a BWI Manesty Beta Press (Thomas Eng, Hoffman Estate, Ill.) at the speed of 500 tablets per minute for a target tablet hardness of 16 to 18 Kp.

### (ii) METHOCEL® E10M prepared using direct compression (DC) method

Niacin granular, METHOCEL® E10M, Povidone K90, and stearic acid were weighed according to the designated formulas outlined in Table 1 and then added into an 8 qt blender (LB-9322, Petterson Kelly, East Stroudsburg, Pa.), and blended 10 min. The well-blended mixture was compressed into tablets using a BWI Manesty Beta Press (Thomas Eng, Hoffman Estate, Ill.) at the speed of 500 tablets per minute for a target tablet hardness of 16 to 18 Kp.

### (iii) METHOCEL® K15M prepared using WG method

Niacin USP, METHOCEL® K15M, and Povidone K90 were weighed according to the designated formulas outlined in Table 1 and granulated in a high shear granulator utilizing deionized water as the granulating solution. The wet granules were dried, milled, and then blended with extragranular METHOCEL® K15M and stearic acid. The final well-blended mixture was compressed into tablets using a BWI Manesty Beta Press (Thomas Eng, Hoffman Estate, Ill.) at the speed of 500 tablets per minute for a target tablet hardness of 16 to 18 Kp.

### (iv) METHOCEL® K15M prepared using DC method

Niacin granular, METHOCEL® K15M, Povidone K90, and stearic acid were weighed according to the designated formulas outlined in Table 1 and then added into an 8 qt blender (LB-9322, Petterson Kelly, East Stroudsburg, Pa.), and blended 10 min. The well-blended mixture was compressed into tablets using a BWI Manesty Beta Press (Thomas Eng, Hoffman Estate, Ill.) at the speed of 500 tablets per minute for a target tablet hardness of 16 to 18 Kp.

Analysis included process machine tooling changes; variation in polymer levels (see Table 1); interchange of wet granulation, direct compression and roller compaction methods; variation in PVP levels; changes in tablet-hardness; weight variation (+/−5%); reproducibility; tableting speed variation and tablet stability (release rate after storage, moisture absorption, etc.) Targeted drug release profile was achieved for the three following formulations: (i) METHOCEL® E-10M wet granulation, (iii) METHOCEL® K-15M wet granulation, and (iv) METHOCEL® K15M direct compression. The three formulations further demonstrated favorable stability results following a three month stability study.

Direct compression tablets using METHOCEL® K-15M were selected as a preferred embodiment for further analysis due to economic and stability advantages identified in the analysis described above. Accordingly, with respect to the reformulated 1000 mg niacin DC tablet, evaluation was further made with respect to the impact of granulation size; particle size distribution of each component, bulk and tap density of each component; different lots of each component; content uniformity; Hauser and Carr indices; flowability; compressibility and friability. Table 2 outlines the specific primary materials used in various experimental formulations. DMF is a Drug Master File.

TABLE 2
Materials Used in Niacin ER 1000 mg DC Tablets

| Material | Regulatory Status | Manufacturer |
|---|---|---|
| Niacin Granular, USP | DMF | Lonza, Ltd, |
| Methocel ® K-15M | No DMF - material | The Dow Chemical |
| | conforms to USP/NF | Company |
| Plasdone ® K-90 | No DMF - material | ISP,, New Jersey 07470 |
| | conforms to USP/NF | USA |
| Stearic acid | No DMF - material | Witco Corporation |
| | conforms to USP/NF | |

Table 3 illustrates reformulated test 1000 mg DC tablets containing various levels of excipients and associated physical qualities. These formulations were prepared as described above with the w/w % of each component as described in Table 3.

TABLE 3

| | | w/w (%) | | | | |
|---|---|---|---|---|---|---|
| Item No. | Component | 1280 mg | 1260 mg | 1240 mg | 1220 mg | 1200 mg |
| 1 | Niacin Granular | 78.13 | 79.37 | 80.6 | 82 | 83.33 |
| 2 | Methocel ® K-15M | 18.18 | 16.9 | 15.6 | 14.2 | 12.79 |
| | Premium | | | | | |
| 3 | Povidone | 2.7 | 2.74 | 2.8 | 2.8 | 2.88 |
| 4 | Stearic acid | 1 | 1 | 1 | 1 | 1 |
| | Total | 100 | 100 | 100 | 100 | 100 |

Niacin ER 1000 mg Tablets

| | | | | | |
|---|---|---|---|---|---|
| Hardness (kP) | 18 (16-22) | 18 (16-22) | 18 (16-22) | 18 (16-22) | 18 (16-22) |
| (Target hardness) | | | | | |
| Thickness (mm) | 8.4 (8.0-9.0) | 8.4 (8.0-9.0) | 8.4 (8.0-9.0) | 8.4 (8.0-9.0) | 8.4 (8.0-9.0) |
| (Target thickness) | | | | | |
| Friability (%) | <1 (0-0.5) | <1 (0-0.5) | <1 (0-0.5) | <1 (0-0.5) | <1 (0-0.5) |
| (Target friability) | | | | | |

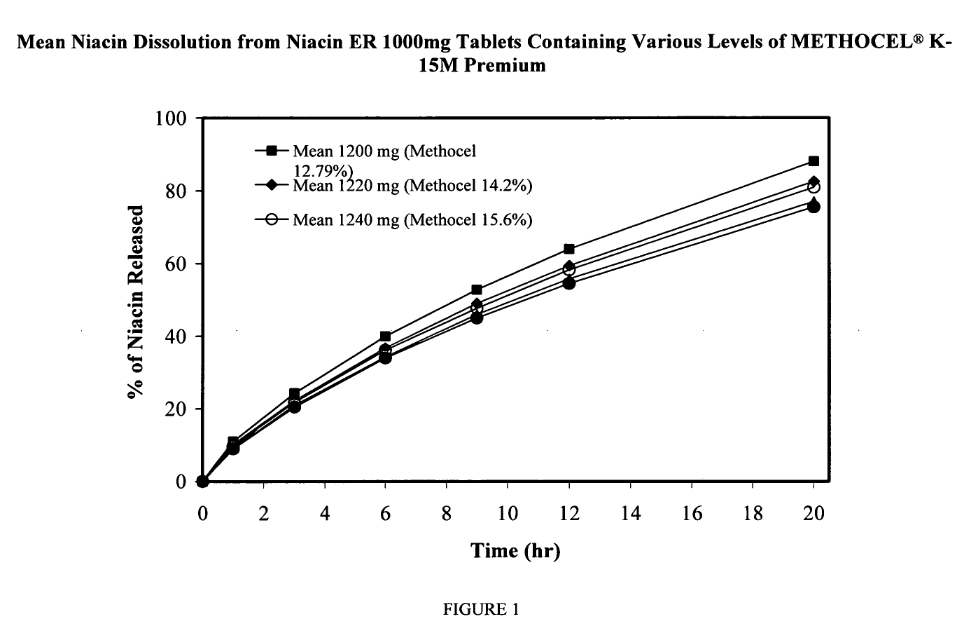

FIG. 1 provides an example comparison of the dissolution profiles for the formulations illustrated in Table 3.

Table 4 illustrates dissolution and bioavailability data of multiple 1000 mg experimental niacin formulations versus 500 mg Niaspan® in clinical studies. Dissolution was calculated using USP Apparatus 1, with 900 mL deionized water, at 100 rpm (basket method) at 37° C.

TABLE 4

| | | Dissolution time | | | | | | BE results for NUA | | | |
|---|---|---|---|---|---|---|---|---|---|---|---|
| Dose | Batch No. | 1 hr | 3 hr | 6 hr | 9 hr | 12 hr | 20 hr | Ratio | Confidence interval | | BE |
| Edison | | | | | | | | | | | |
| Niaspan 500 | reference | 10.6 | 23 | 36.7 | 48.6 | 60 | 83 | | | | |
| DC1220 | ERN-3 | 9.2 | 21 | 36.7 | 49.2 | 60 | 84 | 123.09* | 112.52 | 134.65 | Fail |
| DC1240 | ERN-1 | 9.9 | 22 | 36.6 | 48.5 | 59 | 84 | 110.5* | 101.1 | 120.78 | Pass |
| DC1240 | ERN-2 | 9.3 | 21 | 35.6 | 47 | 57 | 75 | 105.06* | 96.06 | 114.9 | Pass |
| Niaspan 500 | | 10.9 | 24 | 39.4 | 51.1 | 61 | 81 | | | | |
| Hollywood | reference | | | | | | | | | | |
| DC1280 | Test 1 | 8 | 19 | 32 | 43 | 52 | 74 | 80.18** | 72.54 | 88.63 | Fail |
| WG1300 | Test 2 | 9 | 19 | 32 | 44 | 54 | 74 | 80.78** | 73.1 | 89.27 | Fail |
| WG1280 | Test 3 | 10 | 21 | 34 | 45 | 56 | 77 | 80.73** | 72.97 | 89.32 | Fail |

Hollywood niacin repeated dissolution results

| | | | | | | | |
|---|---|---|---|---|---|---|---|
| DC1220 | ERN-3 | 9.3 | 21.0 | 35.0 | 47.1 | 57.6 | 80.7 |
| DC1240 | ERN-1 | 9.3 | 21.2 | 35.1 | 46.8 | 56.9 | 78.0 |
| DC1240 | ERN-2 | 8.8 | 19.9 | 33.3 | 44.8 | 54.9 | 75.5 |

All clinical doses were 2000 mg i.e 4 × 500 mg or 2 × 1000 mg

Reproducibility of the reformulated 1000 mg niacin ER direct compression tablets was investigated by varying the following parameters:

Formulation parameters:

Viscosities and hydroxypropoxyl content of METHOCEL® K-15 MP CR

Particle size of METHOCEL® K-15M

Particle size of niacin granular

Stearic acid content

Sieving of PVP K-90

Processing parameters

Mixing sequences and time

Tablet hardness

Tableting speed

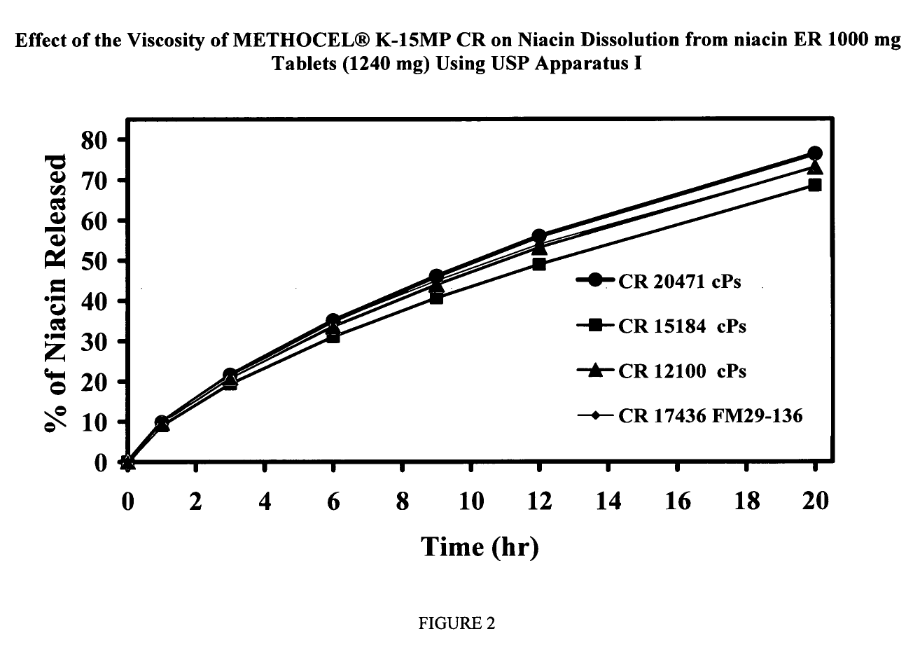

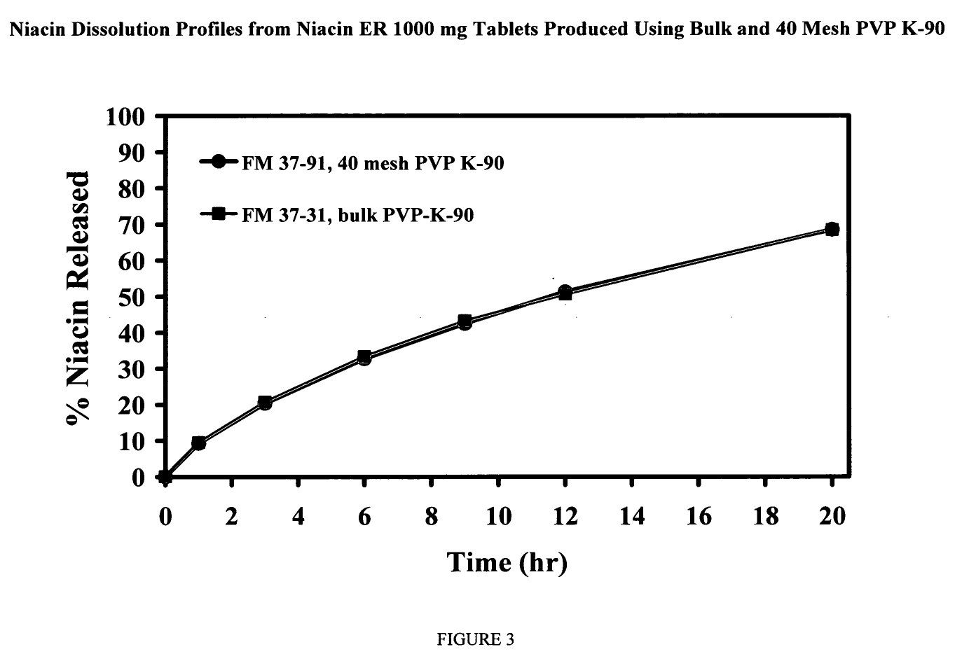

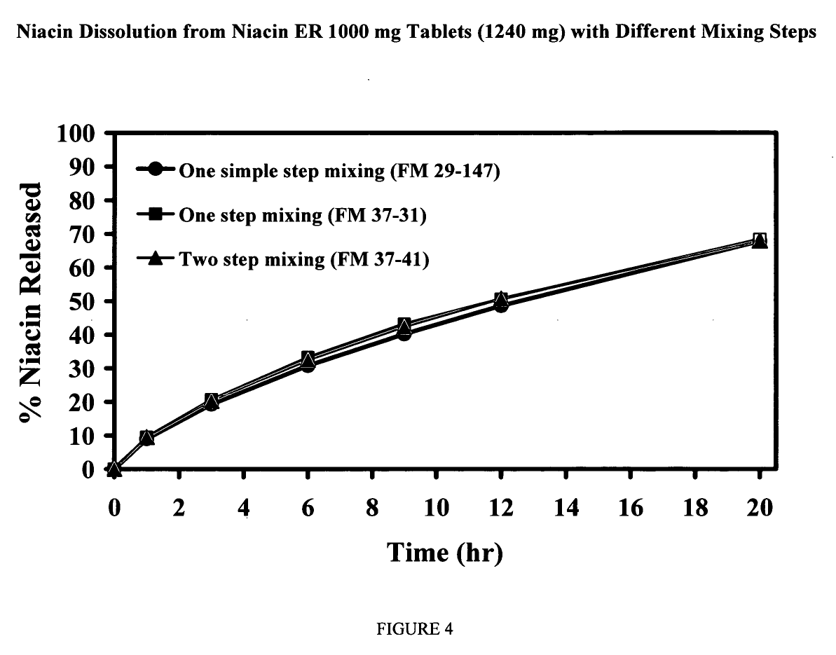

Table 5 below and FIGS. 2-4 illustrate data generated during the reproducibility studies described above.

TABLE 5
Niacin Dissolution from 1000 mg niacin ER tables containing
Various Sizes of Niacin Granular (1240 mg) using USP Apparatus
1 (specifications described above).

| Niacin | | | | | | | |
|---|---|---|---|---|---|---|---|
| Granular | Batch No. | 1 | 3 | 6 | 9 | 12 | 20 |
| | Niaspan 500 | 10.3 | 23 | 38.1 | 51.3 | 62.7 | 86.6 |
| 40-60 mesh | | 9.1 | 20.3 | 32.9 | 43.1 | 51.4 | 69.8 |
| 60-80 mesh | | 10.1 | 21 | 33.2 | 43.4 | 52 | 70.6 |
| 80-100 mesh | | 10.5 | 21 | 32.8 | 42.6 | 51.1 | 70.1 |
| 100-270 mesh | | 11 | 22 | 34.2 | 44.4 | 53.2 | 72.3 |

Upon completing the analysis of the variables above, applicants found no significant difference in niacin dissolution from the tablets made when the following variables were changed: the viscosity and hydroxypropoxyl substitution of METHOCEL® K-15M premium (CR), niacin granular with different particle sizes, sieving PVP K-90 through 40 mesh screen, stearic acid content from 0.5% to 2.0%, mixing steps, and mixing time. The larger particle size of METHOCEL® K-15M premium (CR) and tablet hardness (especially lower than 8 kp) increased the niacin dissolution. The smaller particle size of niacin granular and METHOCEL® K-15M premium CR exhibited higher compressibility. The ejection force decreased significantly as the stearic acid content increased in the formulations. A higher tablet hardness was achieved with higher compression force and ejection force and a higher compression force was needed to get the target tablet hardness (18 Kp) when the tableting speed was increased.

According to the above, the present invention encompasses a wet granulation or direct compression 1000 mg niacin extended-release (ER) tablet formulation comprising:
(a) about 70% to about 92% w/w of niacin;
(b) about 7% to about 25% w/w of a release-retarding agent having a methoxyl degree of substitution of about 1.2 to about 2.0 and a hydroxypropoxyl molar substitution of about 0.1 to about 0.3;
(c) about 0.1% to about 4.3% w/w of a binder, and
(d) about 0.5% to about 1.5% w/w of a lubricant.

In a preferred embodiment, the formulation is made using a direct compression method.

Because the 1000 mg extended-release niacin formulations of the present invention are bioequivalent to two 500 mg NIASPAN® tablets, they would be expected to share both the same efficacy and toxicity profile. Thus, administration of 1000 mg extended-release niacin formulations of the present invention can provide similar treatment benefits to that of two 500 mg NIASPAN® without giving rise to treatment-limiting hepatotoxicity or treatment-limiting elevations in uric acid or glucose levels to an extent which would require the use of the formulation of the invention to be discontinued. Toxicity problems associated with sustained release niacin formulations are well known to those skilled in the art. See for example “A comparison of the Efficacy and Toxic Effects of Sustained- v. Immediate-Release Niacin Hypercholesterolemic Patients”, McKenney et al., JAMA Vol. 271, No. 9, Mar. 2, 1994; and “Hepatic Toxicity of Unmodified and Time-Release Preparations of Niacin”, Rader, et al., The Am. Jour. Of Med., Vol. 92, January 1992, page 77.

Accordingly, one embodiment of the invention comprises administration of the pharmaceutical compositions of the invention to treat a patient in need thereof, wherein the treatment can reduce a serum lipid without generally causing treatment-limiting (i) hepatotoxicity and (ii) elevations in uric acid levels or glucose levels or both, following administration to said patient that would require such treatment to be discontinued when said composition is ingested by said patient once per day. In a further embodiment, administration is once per day, during the evening or at night (for example, after dinner or before bedtime).

### Combination Treatment

The once daily niacin formulations of the present invention can be combined with an HMG-CoA reductase inhibitor. As used herein, “combination therapy” and “combination treatment” encompass administration of a niacin formulation of the present invention and at least one additional active agent in the same or separate pharmaceutical dosage forms. Combination treatment, as used herein, includes simultaneous administration of the active agents and sequential administration of the active agents as part of a treatment regimen.

Examples of HMG-CoA reductase inhibitors include, but are not limited to, lovastatin and related compounds as disclosed in U.S. Pat. No. 4,231,938, pravastatin and related compounds as reported in U.S. Pat. Nos. 4,346,227 and 4,448,979, mevastatin and related compounds as disclosed in U.S. Pat. No. 3,983,140, velostatin and simvastatin and related compounds as discussed in U.S. Pat. Nos. 4,448,784 and 4,450,171, fluvastatin, atorvastatin, rivastatin and fluindostatin (Sandoz XU-62-320). Other HMG-CoA reductive inhibitors include, but are not limited to, pyrazole analogs of mevalonolactone derivatives as disclosed in U.S. Pat. No. 4,613,610, indent analogs of mevalonolactone derivatives as disclosed in PCT application WO 86/03488, 6-[2-(substituted-pyrrol-1-yl)alkyl]pyran-2-ones and derivatives thereof as disclosed in U.S. Pat. No. 4,647,576, Searle's SC45355 (a 3-substituted pentanedioic acid derivative) dichloracetate, imidazole analogs of mevalonolactone as disclosed in PCT application WO 86/07054, 3-carboxy-2-hydroxy-propane-phosphoric acid derivatives as disclosed in French Patent No. 2,596,393, 2,3-di-substituted pyrrole, furan and thiophene derivatives as disclosed in European Patent Application No. 0221025 A14, naphthyl analogs of mevalonolactone as disclosed in U.S. Pat. No. 4,686,237, octahydro-naphthelenes such as disclosed in U.S. Pat. No. 4,499,289, keto analogs of lovastatin as disclosed in European Patent Application No. 0142146 A2, as well as other known HMG-CoA reductase inhibitors, such as those disclosed in GB Patent Nos. 2,205,837 and 2,205,838; and in U.S. Pat. Nos. 5,217,992; 5,196,440; 5,189,180; 5,166,364; 5,157,134; 5,110,940; 5,106,992; 5,099,035; 5,081,136; 5,049,696; 5,049,577; 5,025,017; 5,011,947; 5,010,105; 4,970,221; 4,940,800; 4,866,058; 4,686,237.

Optionally, the pharmaceutical formulations of the present invention can also be administered in combinations with other anti-lipidemic agents. Specific examples of anti-lipidemic agents include, but are not limited to, bile acid sequestrants, e.g., cholestyramine, colestipol DEAESephadex (Secholex.RTM. and Polidexide.RTM.), probucol and related compounds as disclosed in U.S. Pat. No. 3,674,836, lipostabil (Rhone-Poulanc), Eisai E5050 (an N-substituted ethanolamine derivative), imanixil (HOE-402) tetrahydrolipstatin (THL), isitigmastanylphosphorylcholine (SPC Roche), aminocyclodextrin (Tanabe Seiyoku), Ajinomoto A J-814 (azulene derivative), melinamide (Sumitomo), Sandoz 58-035, American Cyanimid CL-277,082 and CL-283,546 (disubstituted urea derivatives), neomycin, p-aminosalicylic acid, aspirin, quartemary amine poly(diallyldimethylamm-onium chloride) and ionenes such as disclosed in U.S. Pat. No. 4,027,009, poly(diallylmethylamine) derivatives such as disclosed in U.S. Pat. No. 4,759,923, omega-3-fatty acids found in various fish oil supplements, fibric acid derivatives, e.g., gemfibrozil, clofibrate, bezafibrate, fenofibrate, ciprofibrate and clinofibrate, and other known serum cholesterol lowering agents such as those described in U.S. Pat. No. 5,200,424; European Patent Application No. 0065835A1, European-Patent No. 164-698-A, G.B. Patent No. 1,586,152 and G.B. Patent Application No. 2162-179-A.

Further, a pharmaceutical formulation of the present invention can be administered in combination with a flush-inhibiting agent. Flush-inhibiting agents include, but are not limited to, nonsteroidal anti-inflammatory drugs such as aspirin and salicylate salts; propionic acids such as ibuprofen, flurbiprofen, fenoprofen, ketoprofen, naproxen, sodium naproxen, carprofen and suprofen; indoleacetic acid derivatives such as indomethacin, etodolac and sulindac; benzeneacetic acids such as aclofenac, diclofenac and fenclofenac; pyrroleacetic acids such as zomepirac and tolmectin; pyrazoles such as phenylbutazone and oxyphenbutazone; oxicams such as piroxicam; and anthranilic acids such as meclofenamate and mefenamic acid.

A flush-inhibiting agent can also be a prostaglandin D2 receptor antagonist including, but not limited to, the compounds disclosed in the U.S. patent Publication Nos. 2004/0229844 and 2005/0154044. A preferred prostaglandin D2 receptor antagonist is MK-0524 (Merck & Co.).

### Delayed-Release

The present invention encompasses delayed-release dosage forms. As used herein, “delayed-release” means that little or no release occurs for a period of time after administration to a patient (i.e., a lag time). A niacin formulation of the present invention can be provided in delayed-release form as the only active agent in a pharmaceutical composition or as one of a plurality of active agents in a pharmaceutical dosage form (the other active agent(s) may or may not be delayed-release). Thus, for example, a pharmaceutical composition can compromise an immediate-release flush-inhibiting agent component combined with a delayed-release niacin component. For example, upon administration of a pharmaceutical composition of the invention to a patient, the immediate-release flush-inhibiting agent releases immediately and the delayed-release niacin component releases after a lag time (e.g., at least about 30 minutes to about 40 minutes).

Delayed-release can be provided using materials and methods well-known in the art. These materials and methods include the following. Single-unit, capsular drug delivery systems, which include an insoluble capsule housing a drug and a plug. The plug is removed after a predetermined lag time due to swelling, erosion, or dissolution. The Pulsincap® system (Scherer DDS, Ltd) is an example of such a system, wherein the body is closed at the open end with a swellable hydrogel plug. Upon contact with dissolution medium or gastro-intestinal fluids, the plug swells, pushing itself out of the capsule after a lag time. This is followed by a rapid drug release. The lag time can be controlled by manipulating the dimension and the position of the plug. See, e.g., WO 90/09168; Wilding et al, Pharm Res. 1992; 9:654-657. The plug material can be made of insoluble, but permeable and swellable polymers (e.g., polymethacrylates) (see Krögel I, Bodmeier R, Pharm Res. 1998; 15(3):474-481; Krögel I, Bodmeier R, Pharm Res. 1999; 16(9):1424-1429) erodible compressed polymers (e.g., hydroxypropyl methyl cellulose, polyvinyl alcohol, polyethylene oxide), congealed melted polymers (e.g., saturated polyglycolated glycerides, glyceryl monooleate), and enzymatically controlled erodible polymers (e.g., pectin). The potential problem of variable gastric residence time can be overcome by enteric coating the system such that dissolution only occurs in the higher pH region of small intestine. Saeger H, Virley P. Pulsincap & Mac226: Pulsed-Release Dosage Form. Product information from Scherer DDS, Ltd; 2004.

The Port® System (Port Systems, LLC) is a capsular system based on osmosis, which consists of a gelatin capsule coated with a semipermeable membrane (e.g., cellulose acetate) housing an insoluble plug (e.g., lipidic) and an osmotically active agent along with the drug formulation. Crison et al., Proceed Intern Symp Control Rel Bioact Mater. 1995; 22:278-279. Upon contact with aqueous medium, water diffuses across the semipermeable membrane, resulting in increased inner pressure that ejects the plug after a lag time. The lag time is controlled by coating thickness.

To deliver the drug in liquid form, an osmotically driven capsular system can be used wherein the liquid drug is absorbed into highly porous particles, which release the drug through an orifice of a semipermeable capsule supported by an expanding osmotic layer after the barrier layer is dissolved. See U.S. Pat. No. 5,318,558. The capsular system delivers drug by osmotic infusion of moisture from the body. The capsule wall is made up of an elastic material and possesses an orifice. As the osmosis proceeds, the pressure within the capsule rises, causing the wall to stretch. The orifice is small enough so that when the elastic wall relaxes, the flow of the drug through the orifice essentially stops, but when the elastic wall is distended beyond threshold value, the orifice expands sufficiently to allow drug release at a required rate. Elastomers, such as styrene-butadiene copolymer can be used. See U.S. Pat. No. 5,221,278; U.S. Pat. No. 5,209,746.

The Time Clock® system (West Pharmaceutical Services Drug Delivery & Clinical Research Centre) is a solid dosage form coated with lipidic barriers containing carnuba wax and bees wax along with surfactants, such as polyoxyethylene sorbitan monooleate. Wilding et al., Int J Pharm. 1994; 111:99-102; Niwa et al., J Drug Target. 1995; 3:83-89. This coat erodes or emulsifies in the aqueous environment in a time proportional to the thickness of the film, and the core is then available for dispersion. In a study of human volunteers, it was shown that the lag time was independent of gastric residence time, and the hydrophobic film redispersion did not appear to be influenced by the presence of intestinal enzymes or mechanical action of stomach or gastro-intestinal pH. Gazzaniga et al., Int J Pharm. 1994; 2(108):77-83. The lag time increased with increasing coating thickness.

The Chronotropic® system is a drug-containing core coated by hydrophilic swellable hydroxypropyl methyl cellulose (HPMC), which is responsible for a lag phase prior to release. Gazzaniga et al., Eur J Biopharm. 1994; 40(4):246-250; Gazzaniga et al., Proceed Intern Symp Control Rel Bioact Mater. 1995; 22:242-243; EP 0 572 942. The application of an outer gastric-resistant enteric film can overcome problems relating to the variability in gastric emptying time. Sangalli et al., J Contr Rel. 2001; 73:103-10. The lag time is controlled by the thickness and the viscosity grades of HPMC. The system is suitable for both tablets and capsules. Conte et al., Drug Dev Ind Pharm. 1989; 15(14-16):2583-2596.

A multilayered tablet, containing two active agents, can be formed from a three-layered tablet construction including two active agent-containing layers separated by a drug-free gellable polymeric barrier layer. U.S. Pat. No. 4,865,849; Conte et al., Eur J Pharm. 1992; 38(6):209-212; Krögel I, Bodmeier R, Int J Pharm. 1999; 187:175-184. This three-layered tablet is coated on three sides with in impermeable ethyl cellulose, and the top portion is uncoated. Upon contact with dissolution medium, the dose incorporated into the top layer releases rapidly from the non-coated surface. The second dose releases from the bottom layer after the gelling barrier layer of HPMC erodes and dissolves. The rate of gelling and/or dissolution of the barrier layer controls the appearance of the second dose. The gelling polymers can include cellulose derivatives like HPMC, methyl cellulose, or polyvinyl alcohols of various molecular weights, and coating materials including ethyl cellulose, cellulose-acetate-propionate, methacrylic polymers, acrylic and methacrylic co-polymers, and polyalcohols.

Pulsatile systems with rupturable coatings depend on disintegration of the coating for release of the drug. The pressure necessary for rupture of the coating can be achieved by effervescent excipients, swelling agents, or osmotic pressure. An effervescent mixture of citric acid and sodium bicarbonate can be incorporated in a tablet core coated with ethyl cellulose. Carbon dioxide produced after penetration of water into the core results in release of drug after rupture of the coating. Bussemer T, Bodmeier R, AAPS Pharm Sci. 1999; 1(4 suppl):434 (1999). Lag time increases with increasing coating thickness and increasing hardness of the core tablet.

Highly swellable agents, also called superdisintegrants, can be used to design a capsule-based system comprising a drug, swelling agent, and rupturable polymer layer. U.S. Pat. No. 5,229,131. Examples of superdisintegrants include cross carmellose, sodium starch glycollate, and low substituted hydroxypropyl cellulose. The swelling of these materials results in a complete film rupture followed by drug release. Lag time is a function of the composition of the outer polymer layer. The presence of a hydrophilic polymer such as HPMC reduces the lag time. The system can be used for delivery of both solid and liquid drug formulations.

Multiparticulate systems (e.g., beads or pellets in a capsule) can be used to provide delayed-release of one active agent and delayed, or other type (e.g., immediate) release of a second active agent. See, e.g., U.S. Pat. No. 4,871,549.

The Time-Controlled Explosion System (Fujisawa Pharmaceutical Co., Ltd.) is a multiparticulate system in which drug is coated on non-pareil sugar seeds followed by a swellable layer and an insoluble top layer. Ueda et al., J Drug Targeting. 1994; 2:35-44; Ueda et al., Chem Pharm Bull. 1994; 42(2):359-363; Ueda et al., Chem Pharm Bull. 1994; 42(2):364-367; Hata et al., Int J Pharm. 1994; 110:1-7. The swelling agents can include superdisintegrants like sodium carboxymethyl cellulose, sodium starch glycollate, L-hydroxypropyl cellulose, polymers like polyvinyl acetate, polyacrylic acid, polyethylene glycol, etc. Alternatively, an effervescent system comprising a mixture of tartaric acid and sodium bicarbonate can be used. Upon ingress of water, the swellable layer expands, resulting in rupture of film with subsequent rapid drug release. The release is independent of environmental factors like pH and drug solubility. The lag time can be varied by varying coating thickness or adding high amounts of lipophilic plasticizer in the outermost layer. U.S. Pat. No. 5,508,040.

A Permeability Controlled System is based on a combination of osmotic and swelling effects. The core contains the drug, a low bulk density solid and/or liquid lipid material (e.g., mineral oil) and a disintegrant. The core is then coated with cellulose acetate. Upon immersion in aqueous medium, water penetrates the core displacing lipid material. After the depletion of lipid material, internal pressure increases until a critical stress is reached, which results in rupture of the coating. U.S. Pat. No. 5,229,131.

Another system is based on a capsule or tablet composed of a large number of pellets consisting of two or more pellets or parts (i.e., populations). Schultz P, Kleinebudde P. J Contr Rel. 1997; 47:181-189. Each pellet has a core that contains the therapeutic drug and a water-soluble osmotic agent. Water-permeable, water-insoluble polymer film encloses each core. A hydrophobic, water-insoluble agent that alters permeability (e.g., a fatty acid, wax, or a salt of fatty acid) is incorporated into the polymer film. The rate of water influx and drug efflux causes the film coating of each population to differ from any other pellet coating in the dosage form. The osmotic agents dissolve in the water causing the pellets to swell, thereby regulating the rate of drug diffusion. The effect of each pellet population releasing its drug content sequentially provides a series of releases of drug from a single dosage form. The coating thickness can be varied amongst the pellets.

Osmotically active agents that do not undergo swelling can also be used to provide delayed-release. Schultz et al., J Contr Rel. 1997; 47:191-199; U.S. Pat. No. 5,260,069. The pellet cores consist of drug and sodium chloride. The cores are coated with a semipermeable cellulose acetate polymer. This polymer is selectively permeable to water and is impermeable to the drug. Lag time increases with increase in coating thickness and higher amounts of talc or lipophilic plasticizer in the coating. Sodium chloride facilitates fast release of drug. In absence of sodium chloride, a sustained release can be obtained after the lag time due to a lower degree of core swelling that resulted in generation of small fissures.

A system containing a core of drug and osmotically active agent (sodium chloride) coated with an insoluble permeable membrane can be used to provide delayed-release. U.S. Pat. No. 5,260,068 The coating materials include different types of poly (acrylate-methacrylate) co-polymers and magnesium stearate, which reduces water permeability of the membrane, thus allowing for use of thinner films. Thicker films are to be avoided because they may not rupture completely. Using ethyl cellulose as a coating material, it is possible to affect a lag time for the enteric polymer to achieve rupture after a predetermined time. Bodmeier et al., Pharm Res. 1996; 13(1):52-56.

The permeability and water uptake of acrylic polymers with quaternary ammonium groups can be influenced by the presence of different counter-ions in the medium. Beckert et al., Proceed Int'l Symp Control Rel Bioact Mater. 1999; 26:533-534. Several delivery systems based on this ion exchange have been developed. Eudragit RS 30D is a preferred polymer for this purpose because it contains a positively polarized quaternary ammonium group in the polymer side chain, which is accompanied by negative hydrochloride counter-ions. The ammonium group is hydrophilic and facilitates the interaction of polymer with water, thereby changing its permeability and allowing water to permeate the active core in a controlled manner. The pellets can be coated with EUDRAGIT RS30D® (10% to 40% weight gain) in four different layer thicknesses. Lag time correlates with film thickness. The drug permeability of the EUDRAGIT film depends on the amount of sodium acetate in the pellet core. After the lag time, interaction between the acetate and polymer increases the permeability of the coating such that the entire active dose is liberated within a few minutes. Guo X. Physicochemical and Mechanical Properties Influencing the Drug Release From Coated Dosage Forms. Doctoral Thesis. The University of Texas at Austin; 1996.

A Sigmoidal Release System includes pellet cores comprising drug and succinic acid coated with ammonio-methacrylate copolymer USP/NF type B. Narisawa et al., Pharm Res. 1994; 11(1):111-116. The lag time is controlled by the rate of water influx through the polymer membrane. The water dissolves succinic acid and the drug in the core. The acid solution in turn increases permeability of the hydrated polymer film. In addition to succinic acid, acetic acid, glutaric acid, tartaric acid, malic acid, or citric acid can be used. The increased permeability can be explained by improved hydration of film, which increases free volume. These findings were used to design a coated delivery system with an acid-containing core. Narisawa et al., Pharm Res. 1994; 11(1): 111-116; Narisawa et al., J Contr Rel. 1995; 33:253-260. The in-vitro lag time correlated well with in-vivo data when tested in beagle dogs. Narisawa et al., J Contr Rel. 1995; 33:253-260.

The present invention encompasses a pharmaceutical composition comprising a niacin formulation of the present invention in a delayed-release form combined with a flush-inhibiting agent. The delayed-release niacin and the flush-inhibiting agent can be provided in one dosage from or separate dosage forms. Thus, for example, the pharmaceutical composition can comprise a solid dosage form having an outer, immediate-release flush-inhibiting agent component and an inner, delayed-release niacin component. In a preferred embodiment, the flush-inhibiting agent is released about 30 minutes to about 40 minutes before the niacin is released.

The following examples serve to better illustrate, but not limit, multiple embodiments of the invention.

### EXAMPLE 1

The following formulation was used in this example:

TABLE 6

| Ingredient | Mg/Tablet | % w/w | Functionality |
|---|---|---|---|
| Niacin granular, USP | 1000 mg | 80.65 | Active drug |
| (NLT 85% (w/w) for | | | |
| sieve fraction 100-425 μm | | | |
| and NMT | | | |
| 10%(w/w) for dust | | | |
| <100 μm) | | | |
| METHOCEL ® K- | 193.1 mg | 15.57 | Release-retarding |
| 15M Premium | | | agent |
| Povidone K-90, USP | 34.50 mg | 2.78 | Binder |
| Stearic Acid, NF | 12.4 mg | 1.00 | Lubricant |
| Total | 1240 mg | 100.0 | — |

Preferably, where METHOCEL® K-15M Premium is employed, the particle size specification for METHOCEL® K-15M Premium is that a minimum of 90% passes through a 100 mesh US standard sieve. For METHOCEL® K-15M Premium CR, preferably a minimum of 99% passes through a 40 mesh US standard sieve, and a minimum of 90% passes through a 100 mesh US standard sieve.

For a 20 kg batch size, delumped niacin granular and the excipients were weighed according to the above formula and then added into an 8-quart blender and blended for 10 minutes at 24 rpm. In particular, a 12 mesh (1.68 mm) screen was selected to delump the METHOCEL® K-15M and stearic acid and a 16 mesh (1.19 mm) screen was selected to delump the niacin granular and Povidone K-90 (optionally sieved, milled, or both). The resultant granular composition was directly compressed into tablets using a BWI Manesty Beta Press with a 19 mm length oval tooling at 30 kN. Tablet hardness (i.e., the compressive strength of the tablet, as measured by standard compression testing methods known to those skilled in the art) was controlled within a range of 16 kP (kilopound) to 22 kP using a standard tablet hardness tester for a target tablet hardness of 18 kP. Optionally, the stearic acid or povidone can be screened through a mesh screen, such as a 40 mesh screen, and mixing steps (one or two) and mixing time (10, 15 or 20) can be varied in alternate embodiments.

The resulting compressed tablets were coated with a 2% weight gain color coat of OPADRY® Orange 03B93199. The coating conditions were as follows:

TABLE 7

| Control/Test Characteristic | Batch Characteristics |
|---|---|
| Batch Size | 12,097 tablets |
| Starting Core Weight (mg) | 1236.3 mg |
| Final Coated Tablet Weight (mg) | 1286.3 mg |
| Vector Hi Coater (Vector Corp., | HC-48/60 |
| Marion, IA) Model # | |
| Equipment # | 002852 |
| Gun to Bed Distance | 6½″ |
| # of Spray Guns | 2 |
| Nozzle Size | 1.2 mm |
| Atomization Air | 150 L/min |
| Pattern Air | 75 L/min |
| Process Air Volume | 170 CFM |
| Spray Rate 60 g/min | 59-64 g/min |
| Pan Speed (10 RPM) | 10 rpm |
| Inlet Temperature TBD | 67.9-72.2° C. |
| Exhaust Temperature 43° C. | 42.0-44.8° C. |
| Weight Gain: 50 mg | 51.1 |
| Spray Time (Report) | 87 minutes |

The coated niacin 1000 mg direct compression tablets were found to be stable for three months at 40° C./75% relative humidity (RH) and 25° C./60% RH by comparing the niacin assay, niacin dissolution, moisture of the tablets and the physical appearance of the coated tablets prior to and following the stability study.

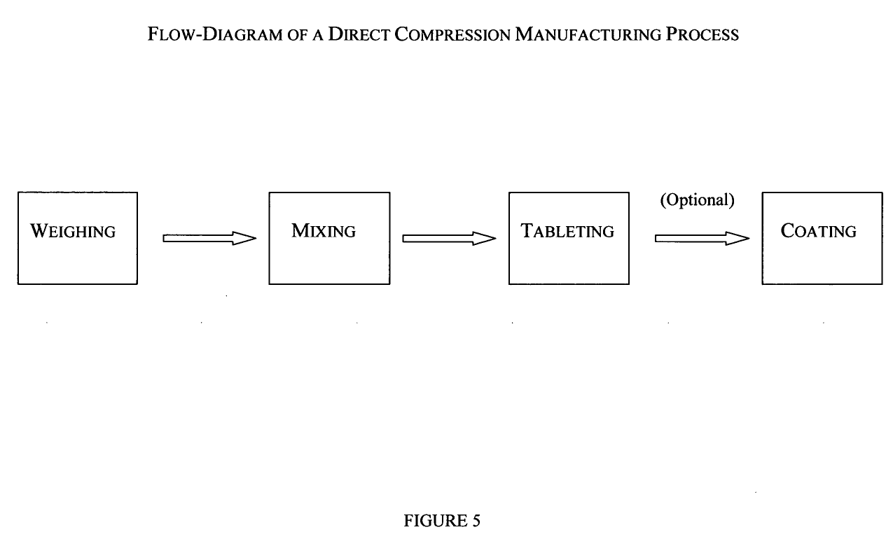

FIG. 5 illustrates a flow-diagram of a direct compression manufacturing process for preparing the tablet formulations in accordance with an embodiment of the invention.

Unless otherwise indicated, the 1000 mg extended-release niacin formulations of the present invention described in the following examples were prepared in accordance with Example 1.

### EXAMPLE 2

Using the processes described herein, 500 mg and 750 mg extended-release direct compression tablets (coated or uncoated) can be prepared having content concentrations illustrated in Tables 8 and 9 below.

TABLE 8
500 MG TABLETS

| Ingredient | Mg/Tablet | % w/w | Functionality |
|---|---|---|---|
| Niacin granular, USP | 500 mg | 70.47 | Active drug |
| (NLT 85% (w/w) for | | | |
| sieve fraction 100-425 μm | | | |
| and NMT | | | |
| 10%(w/w) for dust | | | |
| <100 μm) | | | |
| METHOCEL ® K- | 185.2 mg | 26.1 | Release-retarding |
| 15M | | | agent |
| Povidone K-90, USP | 17.2 mg | 2.42 | Binder |
| Stearic Acid, NF | 7.1 mg | 1.00 | Lubricant |
| Total | 709.5 mg | 100.0 | — |

TABLE 9
750 MG TABLETS

| Ingredient | Mg/Tablet | % w/w | Functionality |
|---|---|---|---|
| Niacin granular, USP | 750 mg | 77.43 | Active drug |
| (NLT 85% (w/w) for | | | |
| sieve fraction 100-425 μm | | | |
| and NMT | | | |
| 10%(w/w) for dust | | | |
| <100 μm) | | | |
| METHOCEL ® K- | 183.1 mg | 18.9 | Release-retarding |
| 15M | | | agent |
| Povidone K-90, USP | 25.8 mg | 2.66 | Binder |
| Stearic Acid, NF | 9.7 mg | 1.00 | Lubricant |
| Total | 968.6 mg | 100.0 | — |

For the 500 mg and 750 mg tablets, delumped niacin granular and the excipients are weighed according to the component concentrations illustrated in Tables 8 and 9 and then blended in a suitable blender or mixer for an appropriate time to adequately mix the components. The resultant granular compositions can then be directly compressed into tablets using a suitable press, such as the BWI Manesty Beta Press described above, to form a 500 mg or 750 mg tablet strength as desired. Optionally, the 500 mg and 750 mg tablet strength can be coated, such as with a color coat, as is known in the art.

### EXAMPLE 3

### Comparative Incidence of Flushing Between Coated, Extended-Release 1000 mg Niacin Direct Compression Matrix Tablets and 1000 mg NIASPAN®

Method

The study was a randomized, double-blind, double-dummy, single-dose, placebo-controlled, three-way crossover, flush provocation study conducted at a single center. Subjects were also precluded from using aspirin or NSAIDs during the study.

The study included healthy, non-smoking male volunteers between 18 and 70 years old with a body mass index (BMI) of 22 to 31. Subjects were confirmed as healthy by a complete physical exam, medical history, electrocardiogram, and results from clinical laboratory testing conducted at the screening visit or at the first study period admission visit. Subjects were excluded if they had allergy or hypersensitivity to niacin or related derivatives; substance abuse or dependency within the last 3 years; history of migraine headaches, diabetes, gallbladder disease, liver disease, severe hypertension or hypotension, cardiac abnormality, renal disease, or drug-induced myopathy. Subjects could not have taken any prescription medications within 21 days or over-the-counter medications, vitamins, or herbals within 10 days prior to entering the study.

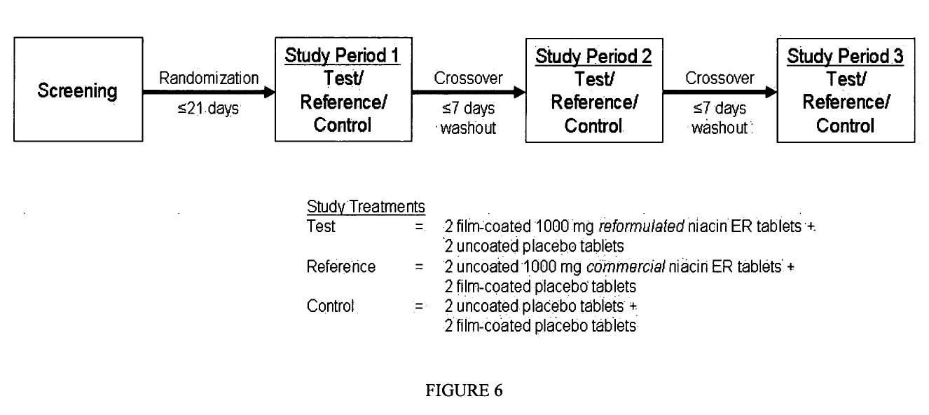

Screening procedures were completed within 21 days prior to clinical admission into Study Period 1 (FIG. 6). For each of the three study periods, subjects remained sequestered from approximately 7:00 AM on Day 1 until the completion of all study procedures on the morning of Day 2 (between 7:00 AM and 10:00 AM). Meal composition and start time was the same for each study period. During each study period, subjects received meals according to specific menus that controlled for niacin and fat content. No concomitant medications, vitamins, or herbals and/or nutritional supplements were permitted during the study.

Study Treatments

The formulations administered in the three study periods are described in FIG. 6. The test treatment used two film-coated 1000 mg tablet formulations of the invention (see Example 1) (Test—reformulated niacin ER tablets), while the reference treatment used two non-coated 1000 mg commercial niacin ER tablets (Reference-NIASPAN®). The control treatment used two non-coated placebo tablets (Control). As this study focused on subject-reported flushing, it was important to completely blind the subjects and study personnel as to the identity of the formulations administered in the treatments. Blinding was accomplished through several methods. In each active treatment, two film-coated placebo or uncoated placebo tablets resembling the active tablets were co-administered with the active tablets so that all subjects received two film-coated and two uncoated tablets regardless of the treatment. Also, study medication was administered to subjects from non-transparent dosing cups and subjects were blindfolded during study drug administration. The placebo-control treatment was included in the study to correct the flush results for an anticipated placebo response.

A single dose of study medication was administered at each study period at approximately 11:00 PM on Day 1, in a crossover manner according to the randomization schedule. There was a minimum washout period of 7 days between each treatment period. Investigators and site personnel were blinded to the treatment assignment scheme, and any site personnel involved in treatment assignment preparation and/or administration was prohibited from collecting or assessing treatment-emergent adverse events.

Each dose was administered orally with 240 mL of water after a low-fat snack. The snack was consumed in its entirety within a 15-minute period before study drug administration. Tablets were either taken together at-once or one immediately following the other, and each subject was instructed to take no longer than 1 minute to complete dosing. Chewing or biting of tablets was prohibited. If a subject required additional water in order to swallow the tablets, additional water was provided in increments of 120 mL. Each subject's mouth was inspected after administration of the study dose to verify consumption of the dose.

Flushing Variables

The primary flushing variable was the occurrence of a subject-reported flushing event or episode. A flushing event or episode was described as one or more of the following concurrent flushing symptoms: redness, warmth, tingling, and itching. During each study period, subjects were prompted to assess the presence or absence of flushing symptoms on an hourly basis for up to 8 hours after study drug administration. The subject was prompted to record start and stop times of the flushing symptom and to rate the intensity (severity) of each symptom by marking a vertical line on a horizontal, 10-centimeter visual analog scale (VAS), anchored from “none” (0) on the left to “intolerable” (100) on the right. The information was recorded in an electronic flushing diary.

Secondary flushing variables included the number of flushing episodes, intensity, and duration of flushing for both overall flushing events and for individual symptoms of flushing (redness, warmth, tingling, and itching). Each subject rated overall intensity of the first flushing event or episode, defined as beginning at the start time of the first of one or more concurrent flushing symptoms to occur in a study period. The end time of the flushing episode was defined as the last stop time of one or more concurrent flushing symptoms occurring in that episode that was also followed by a symptom-free period lasting a minimum of 30 minutes.

Statistical Analysis

It was determined that a sample size of 144 subjects would be required to demonstrate a statistically significant difference in flush incidence between treatments at an alpha (α) of 5% using the McNemar's test (nQuery Advisor®, version 5.0). In order to assure that an adequate number of subjects would complete the study and provide evaluable data from at least two treatments, subjects that discontinued early were replaced.

The primary efficacy assessment (incidence of flush) was compared between treatment groups using McNemar's test of equality of paired proportions. The primary comparison was between the Test and Reference formulations of niacin ER among subjects who received at least one dose of study medication in at least two study periods. Comparisons between niacin and placebo were also performed. Secondary assessments were compared using either McNemar's test (for categorical variables) or the matched pair t-test. All comparisons were two-tailed and conducted at alpha (α)=0.05.

Results

A total of 156 subjects were enrolled in this study and received at least one dose of study medication. Their mean age was 33.5 years, and their mean BMI was 26.2. A summary of subject demographics is presented below in Table 10.

TABLE 10
BASELINE SUBJECT DEMOGRAPHICS

| | | Subjects |
|---|---|---|
| Parameter | | (N = 156) |
| Gender | Male | 156 (100%) |
| Race/Ethnicity | Caucasian | 124 (79.5%) |
| | Black | 14 (9.0%) |
| | Hispanic | 10 (6.4%) |
| | Asian | 2 (1.3%) |
| | Other | 6 (3.8%) |
| Age (y) | Mean | 33.5 |
| | SE | 13.1 |
| Height (in) | Mean | 71.2 |
| | SE | 2.8 |
| Weight (lbs) | Mean | 188.8 |
| | SE | 26.9 |
| BMI | Mean | 26.2 |
| | SE | 2.9 |

BMI = body mass index

All 156 subjects received study medication in Period 1, 143 subjects (92%) received study medication in Period 2, and 131 subjects (84%) received study medication in Period 3. A total of 130 subjects (83%) completed dosing in all three periods. Twenty-six subjects (17%) prematurely discontinued from the study: 8 (5%) withdrew consent, 3 (2%) were lost to follow-up, 2 (1%) had an adverse event, 2 (1%) had protocol violations, 1 (1%) had a positive drug screen, and the remaining 10 (6%) withdrew for “other” reasons. Of the subjects who prematurely discontinued from the study, 11 were replaced in order to ensure sufficient power.

Flushing

As intended, flush provocation was achieved, as the flush incidence in the active treatments was approximately four times higher than that of the control treatment. Table 11 depicts the incidence, intensity, and duration of the first flushing event in the intent-to-treat (ITT) population, defined as the subjects who received at least one dose of study medication and completed at least one study period, but did not include subjects that were replaced. The placebo response seen in this study is typical of placebo responses in general.

TABLE 11
INCIDENCE AND OVERALL INTENSITY AND DURATION
OF THE FIRST FLUSHING EVENT IN ITT POPULATION.

| Treatment | | |
|---|---|---|
| | Commercial | |
| Reformulated | Niacin ER | |
| Niacin ER (Test) | (Reference) | Control |

Incidence of Flushing

| | | | |
|---|---|---|---|
| N | 145 | 140 | 140 |
| Incidence (%) | 127 (88%) | 137 (98%) | 33 (24%) |

Intensity of First Flushing Event (VAS)

| | | | |
|---|---|---|---|
| N | 124 | 137 | 33 |
| Mean (SD) | 35.4 (21.67) | 50.1 (24.24) | 17.3 (14.06) |
| Median | 33 | 54 | 16 |
| Min, Max | 0.0, 99.0 | 0.0, 95.0 | 0.0, 60.0 |

Duration of First Flushing Event (min)

| | | | |
|---|---|---|---|
| N | 127 | 137 | 33 |
| Mean (SD) | 125.3 (94.07) | 184.1 (133.24) | 106.5 (119.87) |
| Median | 98 | 168 | 60 |
| Min, Max | 9.0, 473.0 | 5.0, 984.0 | 4.0, 432.0 |

Incidence and overall intensity and duration of the first flushing event in subjects who received at least one dose of study medication in at least two study periods for Test (reformulated niacin ER) vs Reference (commercial niacin ER) (Reference): A) Incidence of first flushing event (p=0.0027); B) Intensity of first flushing event based on VAS; median values depicted [mean values were 35.6±22.78 (min, max: 0.0, 99.0) with Test and 52.8±23.86 (min, max: 0.0, 95.0) with Reference, p<0.001]; C) Duration of first flushing event (min); median values depicted [mean values were 130.3±95.01 (min, max: 9.0, 473.0) with Test and 195.7±136.32 [min, max: 5.0, 984.0] with Reference, p<0.0001].

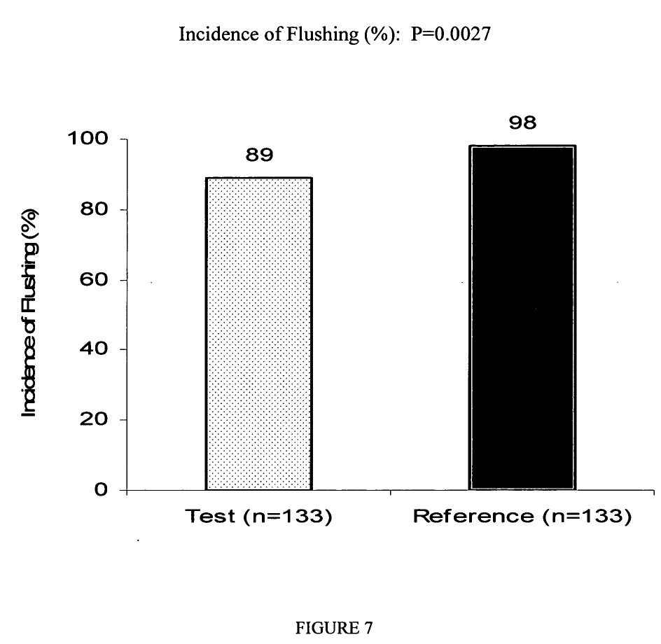

As shown in FIG. 7, for the primary efficacy assessment, among subjects who received at least one dose of study medication in at least two study periods, 118 (89%) subjects experienced flushing during treatment with the Test formulation and 130 (98%) subjects experienced flushing during treatment with the Reference formulation. This difference was statistically significant with a p value of 0.0027.

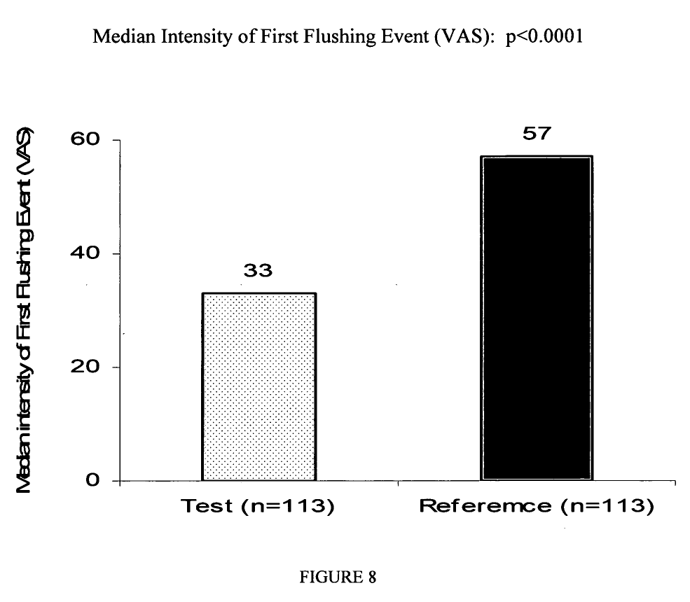

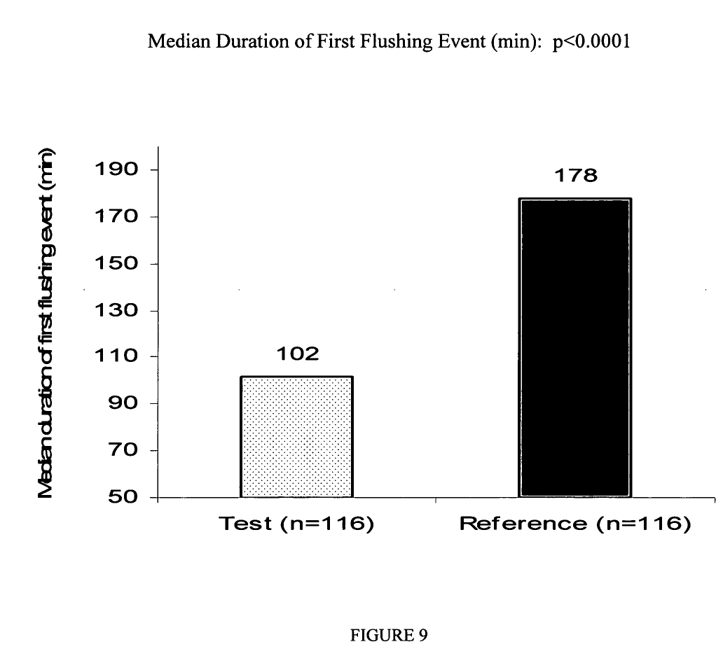

FIGS. 8 and 9 depict the median intensity and duration of the first flushing event. Comparison of the mean values for intensity and duration with their respective medians suggested that the underlying distribution of these data was skewed. The Test treatment resulted in a 42% reduction in median flush intensity (33% reduction in mean flush intensity) and a 43% reduction in median flush duration (33% reduction in mean duration of flush) relative to the Reference treatment. The paired t-test demonstrated statistically significant improvements with the Test treatment for both mean intensity (p<0.0001) and mean duration (p<0.0001) of the first flushing event.

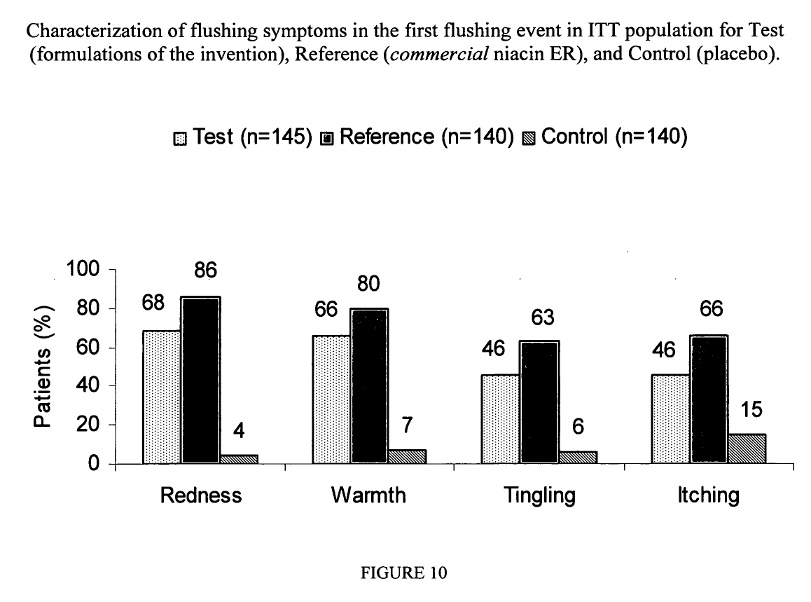

There was a lower incidence of each of the four flushing symptoms (redness, warmth, tingling, and itching) with the Test versus the Reference formulation (FIG. 10). The comparison of the two formulations was significantly different, in favor of the Test formulation for each of the four individual flushing symptoms for the first flushing event using the McNemar's test. Redness occurred in 71% with the Test versus 86% with the Reference formulation (p=0.0016); warmth occurred in 68% with the Test versus 80% with the Reference formulation (p=0.0163); tingling occurred in 47% with the Test versus 62% with the Reference formulation (p=0.0039); itching occurred in 48% with the Test versus 65% with the Reference formulation (p=0.0015).

The data illustrate that the formulations of the invention decrease the incidence, intensity (severity), and duration of flushing compared with the commercially available formulation. Overall, there was a statistically significant 9% reduction in the incidence of flushing with the formulations of the invention (89%) compared with the commercial niacin ER formulation-NIASPAN® (98%), even though the study was designed to provoke flushing by administering a single large (2000 mg) dose to subjects who were treatment-naïve to niacin. Administration of the formulations of the invention in this flush provocation study also resulted in highly statistically significant decreases in flush intensity and duration. Median flush intensity and duration were decreased by 42% and 43%, respectively, relative to the commercial niacin ER treatment. Also, the duration of first flushing event was more than 1 hour shorter with the formulations of the invention.

### EXAMPLE 4

The purpose of this study was to determine the bioequivalence (BE) of the 1000 mg extended-release niacin tablets of the invention (referred to hereinafter as “reformulated” tablets) (Test) versus commercially available 1000 mg NIASPAN® tablets (REF) when administered as a single dose of 2000 mg.

Study Design

The study was a randomized, single-center, open-label, single-dose, two-way crossover study in 44 healthy, nonsmoking male and female volunteer subjects, 40 to 70 years-of-age, inclusive. Drop-outs were not replaced. Each subject received two niacin formulations, Test and REF, in the same single dose of 2000 mg on two separate occasions, with a washout period of at least 10 days between doses. The Test product was reformulated 1000 mg extended-release niacin tablet and the reference product (REF) was 1000 mg NIASPAN® tablet. Each dose was administered with 240 mL of water after a low-fat snack beginning at approximately 22:00 hours (hrs) on Day 1 of each period. Subjects were housed in the study site during each study period (5 days for period 1 and 6 days for period 2) and received meals according to sponsor-provided menus. No other medications, vitamins, herbal or nutritional supplements were permitted during the study.

Serial blood samples were collected from within 30 min prior to dosing out through 24 hrs post dose after dosing at the intervals: −30 min (pre-dosing), 1, 2, 3, 4, 4.5, 5, 6, 7, 8, 10, 12, 14, 16, and 24 hrs (post-dosing). Urine was collected from 24 hrs prior to dosing until 96 hrs after dosing at the intervals: −24 to −18, −18 to −12, −12 to −6, and −6 to 0 hrs (pre-dosing); 0 to 6, 6 to 12, 12 to 18, 18 to 24, 24 to 48, 48 to 72, and 72 to 96 hrs (post-dosing). Plasma was analyzed for niacin, and nicotinuric acid (NUA). Urine was analyzed for niacin, and its metabolites: NUA, N-methylnicotinamide (MNA), and 2-PY (N-methyl-2-pyridone-5-carboxamide).

Niacin is extensively metabolized and plasma concentrations show much higher variability compared to NUA, one of its major metabolites. Hence maximum plasma concentration (Cₘₐₓ) for NUA has been used to determine the rate of niacin absorption. As demonstrated in the NIASPAN® NDA, total urine recovery is a more accurate measure of the extent of absorption than AUC, as AUC is more susceptible to non-linear pharmacokinetics. Therefore the total amount of niacin excreted as niacin and three of its metabolites NUA, MNA and 2PY in urine serves as a measure of the extent of niacin absorption. The primary variables to evaluate NUA bioequivalence defined in the protocol are hence the Cₘₐₓ for NUA and total urinary recovery of niacin and three metabolites (NUA, MNA, and 2PY).

The Test medication consisted of two tablets of reformulated 1000 mg extended-release tablets of the invention. The REF medication consisted of two tablets of 1000 mg NIASPAN® tablets. Treatments were separated by at least 10 days.

Subjects began meals at the same times of each day when they were confined to the clinic during each period. Meals were held at the same for each period, and the entire contents of each meal were required to be consumed. Breakfast, lunch, dinner, and an evening snack began at approximately 07:00, 12:00, 18:00, and 21:45, respectively. The actual meal or snack time for each subject was scheduled relative to the actual dosing time. Subjects were required to drink a minimum of 720 mL of water on Day −1 and 1440 mL of water on Day 1 through 5 in addition to the 240 mL of water given with the study medication on Day 1.

On Day −1, dinner and an evening snack were served. On Days 1 through 5, breakfast, lunch, dinner, and an evening snack were served. The evening snack was consumed within 15 minutes just prior to dosing on Day 1 in each period. On Day 6 in Period 2, no meals were served as subjects were discharged from the clinic after the completion of all clinical procedures.

Evaluation of Pharmacokinetics

a. Plasma Collection and Analysis

Serial blood samples were collected within 30 min prior to dosing through 24 hrs after dosing in each period (15 samples/treatment). Each blood sample was collected into one 10-mL vacutainer containing sodium heparin and was allowed to cool in an ice-chip and water bath for a minimum of 5 min after collection. Samples were centrifuged at 4° C. at approximately 3000 rpm for 15 min to separate the plasma. Each plasma sample was divided into two aliquots, Aliquot A and B, and transferred into two pre-chilled, appropriately labeled polypropylene tubes. Samples were then stored frozen at approximately −20° C.

Niacin and NUA concentrations were analyzed by validated liquid chromatography tandem mass spectroscopy (LC/MS/MS). Niacin and NUA concentrations were obtained from the same injection. The lower limit of quantitation (LLQ) for both niacin and NUA was 2 ng/mL in plasma. Quality control samples were evaluated with each analytical run.

b. Urine Collection and Analysis

Urine was collected for the following intervals: −24 to −18, −18 to −12, −12 to −6, −6 to 0 hrs (prior to dosing), and 0 to 6, 6 to 12, 12 to 18, 18 to 24, 24 to 48, 48 to 72, 72 to 96 hrs after dosing (for a total of 11 collections).

Urine was collected and transferred into plastic containers equipped with tightly fitting lids. Collected urine was kept refrigerated or in an ice-water bath during the collection interval. The collection containers were labeled to identify the subject number and initials, collection interval, and protocol number. The empty containers were weighed to the nearest tenth of a gram (e.g. 100.1 g) and this was written on the container and documented on the lab's source document worksheets. At the end of each interval, the total weight of the container and the collected urine was measured to the nearest tenth of a gram recorded. The weight of the urine was derived by subtracting the weight of the empty container from the total weight of the container plus urine. In some cases, the volume of urine during a given collection interval exceeded the capacity of a single container; therefore a second container was required to obtain a complete urine collection. The start and stop date(s) and times of each urine collection interval were also recorded. Two aliquots (approximately 2.5 mL each) from each collection interval were transferred into two appropriately labeled polypropylene tubes. If more than one container was required during a particular collection interval, the urine from both containers was mixed together before the aliquots were taken. Samples were stored frozen at approximately −20° C. until analysis.

Urine samples were analyzed for concentrations of niacin, NUA, MNA and 2-PY by validated LC/MS/MS. Urine niacin and NUA concentrations were obtained from the same injection while MNA and 2-PY concentrations were obtained from the same injection. In urine the LLQ values were 20 ng/mL for niacin and 200 ng/mL for NUA. MNA and 2PY had LLQ values of 500 ng/mL and 2500 ng/mL respectively. Quality control samples were evaluated with each analytical run.

c. Plasma Pharmacokinetic Parameters and Urinary Recovery

Data from subjects providing sufficient information to calculate PK parameters for at least one treatment were included in the PK analysis. The following PK parameters were calculated for each subject following administration of each treatment:
Cₘₐₓ: the maximum concentration observed
Tₘₐₓ: the time of the maximum observed concentration
AUCₗₐₛₜ: the area under the concentration-time profile from time 0 to the last measurable (non-zero) concentration by the linear trapezoidal rule
AUC_{inf}: the area under the plasma concentration-time profile from time 0 to infinity; calculated as the sum of AUCₗₐₛₜ and Cₜ over λ where Cₜ is the last observed concentration and % is the terminal elimination rate constant obtained from the plot of natural-log concentration versus time plots
T_{1/2}: the apparent terminal half-life; calculated as a ratio of 0.693 over the λ

From the urine data of niacin and its metabolites (NUA, MNA, and 2-PY) the following parameters were computed:
CumXᵤ: cumulative amount of each metabolite recovered from urine from 0 to 96 hrs after dosing.
% Fe: fraction of each metabolite excreted in the urine relative to dose of niacin after correction for baseline recovery and molecular weight in 96 hrs after dosing.
Total % Fe: total fraction of the four metabolites in 96 hrs after dosing.
The % Fe, for each analyte in urine calculated as:

$$\% Fe = \frac{CumXu}{Dose} ⨯ \frac{MW\_of \_Niacin}{MW\_of \_Analyte} ⨯ 100$$

Concentrations below the limit of quantitation were treated as zero. For plasma analysis actual sample collection times were used to compute PK parameters. The amount of niacin and its metabolites recovered in the urine was determined by multiplying each metabolite concentration by the volume of urine collected for each interval. The total amount recovered in urine for each 24 hour interval after dosing was adjusted for baseline by subtracting the amount recovered in the 24 hour pre-dose interval. If any post-dosing measurement was less than baseline the amount was set to zero. The molecular weights of niacin and its metabolites are 123.1, 180.2, 137.1, and 153.1 for niacin, NUA, MNA, and 2-PY, respectively. The sum of % Fe from the four urine analytes, was calculated and designated as total % Fe.

Bioavailability parameters (as described above) were calculated using WinNonlin Linear Mixed Effects Modeling/bioequivalence, Version 5.0.1 (Jul. 26, 2005).

Statistical Analysis

Statistical analyses of the bioavailability parameters calculated above were performed using a SAS® System for Windows™, version 8.2.

Plasma pharmacokinetic parameters (Cₘₐₓ, Tₘₐₓ, T_{1/2}, AUCₗₐₛₜ and AUC_{inf}), their natural log-transformed value (except for Tₘₐₓ and T_{1/2}), and summary statistics (m, mean, std, median, min, max, CV %) were calculated by treatment and period. Plasma concentrations of niacin and NUA are summarized by time and treatment.

For the niacin and NUA PK analysis, it is assumed that the data of the natural log-transformed Cₘₐₓ and AUCₗₐₛₜ follow a normal distribution and are independent between the two treatments. The data were fitted to an ANOVA model with mixed effects using SAS PROC MIXED with treatment, period, and sequence as fixed effects and subject within sequence as a random effect. The Test/REF ratios of Cₘₐₓ and AUCₗₐₛₜ and their corresponding 90% confidence intervals were estimated based on this model.

The mean recovery of niacin and its metabolites from urine was calculated and summarized by treatment and by interval. The CumXᵤ and % Fe of individual components and the total in 96 hrs after dosing were calculated and summarized by treatment.

The 90% confidence intervals (CIs) for the Test/REF mean ratios of total % Fe was calculated by fitting the same ANOVA model as used for plasma PK analysis.

Subjects' demographic variables (age, gender, race, weight, height, and elbow breadth) were summarized by gender. The mean, standard deviation (SD), median, minimum, and maximum of the continuous demographic variables were computed.

Results

Subject disposition is summarized in Table 12. A total of 44 subjects were enrolled in the study after they met the protocol inclusion and exclusion criteria. All the subjects received at least one dose of study medication, and 41 of them completed the study. Forty-four subjects received study medication in Period 1 according to the randomized treatment assignment in the protocol; whereas 41 subjects received study medication in period 2. A total of 3 subjects discontinued from the study. Subject 0012 and 0039 were discontinued in period 2. Subject 0038 withdrew consent in period 2. The numbers of subjects who discontinued from the study were in the range of pre-allowed 10% dropout, and were not considered to affect the results or conclusions of this study.

TABLE 12
Summary of Subject Disposition

| | Subject numbers (N) | Percent (%) |
|---|---|---|
| Enrolled | 44 | 100 |
| Completed study | 41 | 93.2 |
| Received at least one dose | 44 | 100 |
| Received medication at Period 1 | 44 | 100 |
| Received medication at Period 2 | 41 | 93.2 |
| Discontinuation | 3 | 6.8 |

Of the enrolled forty-four subjects, 25 subjects were men and 19 were women. The mean age was 54.5 years; the mean weight was 169.8 pounds; the mean height was 68.0 inches; and the mean elbow breadth was 2.6 inches. Thirty-seven of the subjects were Caucasian, 6 Black, and one was American Indian. The detailed demographics are summarized in Table 13.

TABLE 13
Summary of Subject Demographics

| | | | By Gender | |
|---|---|---|---|---|
| | | All Subjects | Males | Females |
| Characteristic | | (N = 44) | (N = 25) | (N = 19) |
| Age | | | | |
| N | | 44 | 25 | 19 |
| Mean (SD) | | 54.5 (8.1) | 52.8 (8.1) | 56.6 (7.8) |
| Median | | 56 | 52 | 58 |
| Minimum, | | 40.0, 69.0 | 41.0, 69.0 | 40.0, 68.0 |
| Maximum | | | | |
| Gender | | | | |
| Male | N (%) | 25 (56.8) | 25 (100.0) | 0 (0.0) |
| Female | N (%) | 19 (43.2) | 0 (0.0) | 19 (100.0) |
| Race | | | | |
| Caucasian | N (%) | 37 (84.1) | 23 (92.0) | 14 (73.7) |
| Black | N (%) | 6 (13.6) | 2 (8.0) | 4 (21.1) |
| Hispanic | N (%) | 0 (0.0) | 0 (0.0) | 0 (0.0) |
| Asian | N (%) | 0 (0.0) | 0 (0.0) | 0 (0.0) |
| Other | N (%) | 1 (2.3) | 0 (0.0) | 1 (5.3) |
| Height (in) | | | | |
| N | | 44 | 25 | 19 |
| Mean (SD) | | 68.0 (3.9) | 70.2 (3.6) | 65.2 (2.1) |
| Median | | 68 | 70 | 65 |
| Minimum, | | 61.0, 76.0 | 61.0, 76.0 | 61.0, 70.0 |
| Maximum | | | | |
| Weight (lb) | | | | |
| N | | 44 | 25 | 19 |
| Mean (SD) | | 169.8 (20.1) | 180.9 (15.7) | 155.1 (15.2) |
| Median | | 166.5 | 180 | 154 |
| Minimum, | | 133.0, 207.0 | 155.0, 207.0 | 133.0, 190.0 |
| Maximum | | | | |
| Elbow | | | | |
| breadth (in) | | | | |
| N | | 44 | 25 | 19 |
| Mean (SD) | | 2.6 (0.3) | 2.7 (0.2) | 2.5 (0.3) |
| Median | | 2.6 | 2.7 | 2.5 |
| Minimum, | | 2.1, 3.1 | 2.3, 3.1 | 2.1, 3.0 |
| Maximum | | | | |

### Table 13. Summary of Subject Demographics All Subjects By Gender

Males Females

Characteristic (N=44) (N=25) (N=19)

Age

N 44 25 19

Mean (SD) 54.5 (8.1) 52.8 (8.1) 56.6 (7.8) Median 56. 52 58 Minimum, Maximum 40.0, 69.0 41.0, 69.0 40.0, 68.0

Gender

Male N (%) 25 (56.8) 25 (100.0) 0 (0.0) Female N(%) 19 (43.2) 0 (0.0) 19(100.0)

Race

Caucasian N (%) 37 (84.1) 23 (92.0) 14 (73.7) Black N (%) 6 (13.6) 2 (8.0) 4 (21.1) Hispanic N (%) 0 (0.0) 0 (0.0) 0 (0.0) Asian N (%) 0 (0.0) 0 (0.0) 0 (0.0)

Other N (%) 1 (2.3) 0 (0.0) 1 (5.3)

Height (in)

N 44 25 19

Mean (SD) 68.0 (3.9) 70.2 (3.6) 65.2 (2.1) Median 68 70 65 Minimum, Maximum 61.0, 76.0 61.0, 76.0 61.0, 70.0

Weight (lb)

N 44 25 19

Mean (SD) 169.8 (20.1) 180.9 (15.7) 155.1 (15.2) Median 166.5 180 154 Minimum, Maximum 133.0, 207.0 155.0, 207.0 133.0.,190.0

Elbow breadth (in)

N 44 25 19

Mean (SD) 2.6 (0.3) 2.7 (0.2) 2.5 (0.3) Median 2.6 2.7 2.5 Minimum, Maximum 2.1, 3.1 2.3, 3.1 2.1, 3.0

a. Assessment of Bioequivalence

For urine analyses, a specific gravity of 1 g/mL was used to convert urine weights to volumes. This was based on a previous study with NIASPAN® where the mean specific gravity measured in 962 samples was 1.009 g/mL and the maximum specific gravity measured in 962 samples was 1.025 g/mL.

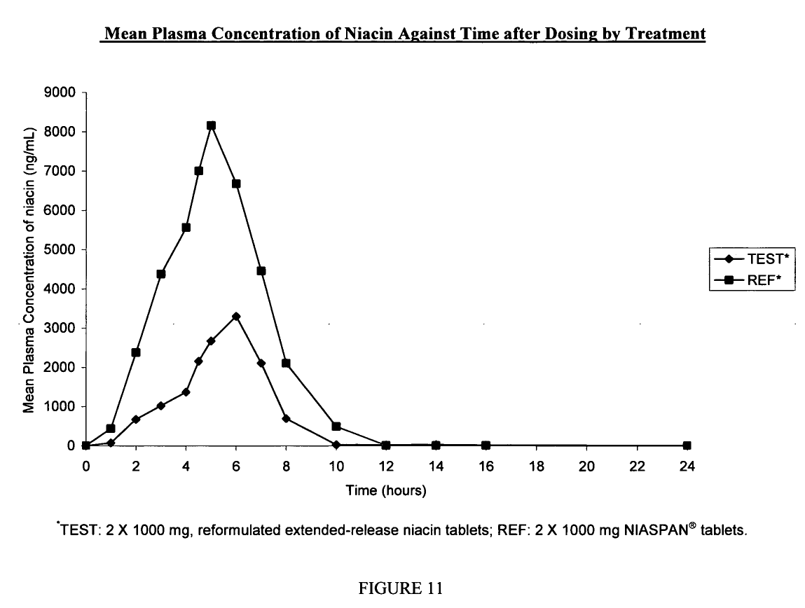

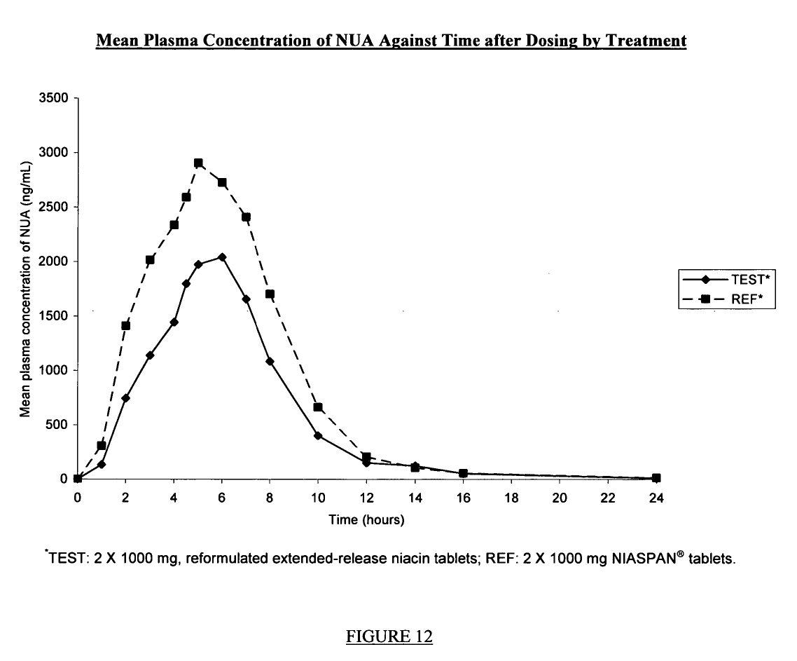

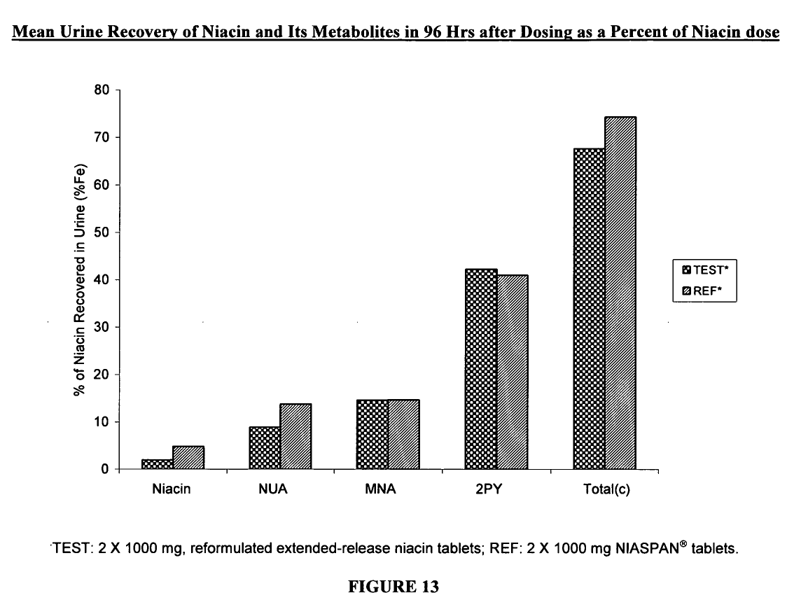

The plots of mean plasma concentrations of niacin and NUA by treatment are shown in FIGS. 11 and 12, respectively. Mean urinary recovery data is shown in FIG. 13.

b. Plasma NUA and Total Amount Excreted in Urine

Table 14 shows the mean (SD) and statistical results for the two primary variables (Cₘₐₓ for NUA and total urinary recovery of niacin and three metabolites) and for NUA AUCₗₐₛₜ. The table gives results of BE analysis with and without the reference treatments for subjects 0001, 0003, and 0014 who had episodes of vomiting following dosing.

Subject 0001 had a vomiting at 7 hrs and 20 min after dosing REF product in period 2. Subject 0003 had two episodes of vomiting at 8 hrs and 34 min, and at 9 hrs and 20 min after dosing REF product in period 2, respectively. Subject 0014 had vomiting at 11 hrs and 20 min after dosing REF product in period 1. The vomiting onset time for all the three subjects was at least 7 hrs and 20 min after dosing. The Tₘₐₓ, for both NUA and niacin were within 6 hrs after doing. Therefore, the vomiting was not considered to affect the PK parameters of these subjects.

TABLE 14
Summary of NUA Plasma Parameters and Total Urinary Recovery

| | | | Excluding subjects | |
|---|---|---|---|---|
| | All subjects | | 0001, 0003, 0014, | |
| Pa- | (N_{Test} = 42; N_{REF} = 43) | | (N_{Test} = 42; N_{REF} = 40) | |
| ram- | | % Ratio | | % Ratio |
| eter | Mean (SD) | (90% CI) | Mean (SD) | (90% CI) |

NUA Cₘₐₓ^{a} (ng/mL)

| | | | | |
|---|---|---|---|---|
| Test | 2621.0 (1335.6) | 66.68 | 2621.0 (1335.6) | 67.41 |
| REF | 3776.2 (1606.2) | (60.41, 73.61) | 3729.7 (1625.8) | (60.87, |
| | | | | 74.64) |

Total Recovery^{a,b} (%)

| | | | | |
|---|---|---|---|---|
| Test | 67.7 (8.4) | 90.93 | 67.7 (8.4) | 90.02 |
| REF | 74.3 (8.3) | (87.62, 94.37) | 74.9 (8.3)^{d} | (86.84, |
| | | | | 93.32) |

NUA AUCₗₐₛₜ^{c} (ng * hr/mL)

| | | | | |
|---|---|---|---|---|
| Test | 12468.3 (6731.8) | 63.99 | 12468.3 (6731.8) | 64.78 |
| REF | 18917.3 (8502.5) | (59.24, 69.12) | 18790.4 (8576.8) | (60.10, |
| | | | | 69.82) |

^{a}Parameters used to define Niacin bioequivalence
^{b}Recovery of niacin, NUA, MNA, and 2PY combined
^{c}N = 42;
^{d}N = 39

As shown in the above table the 90% CI for the mean Test/REF ratio of NUA Cₘₐₓ was out of the bioequivalent range of 80-125%, but the 90% CI for the Test/REF mean ratio of niacin and metabolites recovered from urine were within 80-125%. The results were similar with and without the REF treatments for subjects 0001, 0003, and 0014.

The terminal elimination rate was calculated for each subject by treatment. Mean NUA T_{1/2} were 3.16 and 3.47 hrs, mean NUA Tₘₐₓ were 5.55 and 5.80 hrs, and mean NUA AUC_{inf} were 12510.8 and 18980.8 ng*hr/ml, for Test and REF, respectively.

c. Plasma Niacin

Mean PK parameters for plasma niacin along with statistical analyses are presented in Table 15. The table gives results of BE analysis with and without the REF treatment for subjects 0001, 0003, and 0014. The Test/REF mean ratios of niacin Cₘₐₓ and AUCₗₐₛₜ were less than 100%. The corresponding 90% CI for the ratios were outside the 80-125% interval due to high variability. The results were similar with and without the REF treatments for subjects 0001, 0003, and 0014.

TABLE 15
Summary of Niacin Plasma Parameters

| | | | Excluding subjects | |
|---|---|---|---|---|
| | All subjects | | 0001, 0003, 0014, | |
| Pa- | (N_{Test} = 42; N_{REF} = 43) | | (N_{Test} = 42; N_{REF} = 40) | |
| ram- | | % Ratio | | % Ratio |
| eter | Mean (SD) | (90% CI) | Mean (SD) | (90% CI) |

Niacin Cₘₐₓ (ng/mL)

| | | | | |
|---|---|---|---|---|
| Test | 5210.3 (4969.5) | 34.01 | 5210.3 (4969.5) | 34.93 |
| REF | 12568.5 (9228.5) | (26.22, | 12253.1 (9294.4) | (26.58, |
| | | 44.11) | | 45.90) |

Niacin AUCₗₐₛₜ (ng * hr/mL)

| | | | | |
|---|---|---|---|---|
| Test | 12637.4 (14810.9) | 33.02 | 12637.4 (14810.9) | 33.74 |
| REF | 36307.8 (32486.7) | (26.94, | 35503.2 (32645.0) | (27.31, |
| | | 40.46) | | 41.68) |

Mean T_{1/2} of niacin were 5.46 and 4.42 hrs, mean Tₘₐₓ were 5.56 and 5.55 hrs, and mean AUC_{inf} were 13987.8 and 35296.6 ng*hr/ml, for Test and REF, respectively.

d. Urinary Recovery of Individual Analytes

The mean urine recovery of the individual analytes is given in Table 16.

TABLE 16
Summary of Urinary Excretion of Niacin and its Metabolites

| | | All Subjects | Excluding subjects |
|---|---|---|---|
| | | (N_{Test} = 42, | 0001, 0003, 0014 |
| | | N_{REF} = 42) | (N_{Test} = 42, N_{REF} = 39) |
| | | Mean (SD) | Mean (SD) |
| Niacin Recovery^{a} | Treatment A | 1.94 (1.73) | 1.94 (1.73) |
| | Treatment B | 4.84 (3.79) | 4.94 (3.80) |
| NUA Recovery^{a} | Treatment A | 8.88 (3.54) | 8.88 (3.54) |
| | Treatment B | 13.77 (5.53) | 14.12 (5.52) |
| MNA Recovery^{a} | Treatment A | 14.58 (3.27) | 14.58 (3.27) |
| | Treatment B | 14.67 (3.23) | 14.78 (3.27) |
| 2PY Recovery^{a} | Treatment A | 42.24 (7.08) | 42.24 (7.08) |
| | Treatment B | 41.01 (6.13) | 41.09 (6.31) |

^{a}Recovery as % of niacin dose

As showed in the above table, mean urinary recovery was the highest for 2PY followed by MNA, NUA and niacin.

e. Conclusions of the Bioequivalent Assessment

Bioequivalence was evaluated based on the 90% CIs for mean Test/REF ratios of the NUA Cₘₐₓ and urinary recovery of niacin and its metabolites (Total % Fe). The 90% CIs of Test/REF mean ratio for Total % Fe were within the required BE range of 80-125%, but for NUA Cₘₐₓ were out of the bioequivalent range. The 90% CI of Test/REF mean ratios for supportive measurements including NUA AUCₗₐₛₜ, also fell out of the 80-125% range. Thus. the reformulated 1000 mg ER niacin tablet (Test) shows a lower rate of absorption and a comparable extent of absorption as compared to the NIASPAN® 1000 mg tablet (REF). The Test treatment is not bioequivalent to the REF treatment.

### EXAMPLE 5

The study was designed to determine the bioequivalence of three formulations of 1000 mg extended-release niacin tablets of the invention (referred to hereinafter as “reformulated” tablets) relative to commercially available NIASPAN® 500 mg tablets after a single 2000 mg niacin dose.

Study Design

The study was a randomized, single-center, open-label, single-dose, four-way crossover study in 44 healthy, non-smoking female and male volunteer subjects, 40 to 70 years-of-age, inclusive. Dropouts were not replaced. Each subject received the same dose of oral study medication, 2000 mg niacin, on four separate occasions with a minimum washout period of 10 days between doses. Each subject received two tablets of a 1000 mg ER niacin formulation (ERN-1, ERN-2, ERN-3) and four 500 mg NIASPAN® tablets.

Each dose was administered with 300 mL of water after a low-fat snack beginning at approximately 2200 hours. Subjects received meals according to sponsor-provided menus during each treatment period. No other medications, vitamins, herbal or nutritional supplements were permitted during the study. Blood samples were obtained prior to dosing and at frequent intervals for up to 24 hours after dosing; urine was collected for 24 hours prior to and 96 hours after dosing. Plasma was analyzed for NUA and niacin. Urine was analyzed for niacin and its three major metabolites, NUA, MNA, and 2PY. Subjects were housed during the 5-day study period of each treatment.

Meals controlled for niacin content (breakfast, lunch, dinner, and evening snack) were provided during each treatment period.

The Reference treatment was the commercially available 500 mg NIASPAN® (NSP) formulation that consists of a high-potency granulation (niacin, povidone and hydroxypropyl methylcellulose [HPMC]) that is subsequently blended with stearic acid and additional HPMC before compression into tablets.

The test treatments were three different reformulated 1000 mg NIASPAN® formulations (ERN-1, ERN-2 AND ERN-3) made according to Table 17 below.

TABLE 17

| Component | ERN-3 | | ERN-2 | | ERN-1 | |
|---|---|---|---|---|---|---|
| Niacin Granular, USP | 1000.0 | | 1000.0 | mg | 1000.0 | mg |
| METHOCEL ®, | 173.3 | mg | 193.1 | mg | 193.1 | mg |
| K15M Premium, USP | | | | | | |
| Povidone, USP | 34.5 | mg | 34.5 | mg | 34.5 | mg |
| Stearic Acid NF | 12.2 | mg | 12.4 | mg | 12.4 | mg |
| Total Tablet Weight (mg) | 1220.0 | mg | 1240.0 | mg | 1240.0 | mg |

Niacin granular, METHOCEL® K15M, Povidone K90, and stearic acid were weighed according to the formulas designated in Table 17 above and then added into an 8 qt blender (LB-9322, Petterson Kelly, East Stroudsburg, Pa.), and blended for 10 min. The well-blended mixture was compressed into tablets using a BWI Manesty Beta Press (Thomas Eng, Hoffman Estate, Ill.) at the speed of 500 tablets per minute for a target tablet hardness of 16 to 18 Kp.

All meals and beverages were alcohol- and xanthine-free. Since niacin is available in the normal diet, the study diet was controlled to maintain a niacin intake of approximately 25 mg a day while subjects were confined to the clinic. Each dose was administered at approximately 2200 immediately following a low-fat snack.

Subjects began meals at the same time during each period on all days subjects and were confined to the clinic. Meals were the same for each period, and the entire contents of each meal were to be consumed. Breakfast, lunch, dinner, and an evening snack began at approximately 0700, 1200, 1700, and 2145, respectively. The actual meal or snack time for each subject was scheduled relative to the actual dosing time. Subjects were required to drink a minimum of 720 mL of water on Day −1 and 1440 mL of water on Days 1, 2, 3, 4 & 5 in addition to the 300 mL of water given with the study medication on Day 1.

On Day −1, dinner and an evening snack were served. On Days 1, 2, 3, 4 & 5 breakfast, lunch, dinner, and an evening snack were served. The evening snack was consumed within 15 minutes on Day 1 in each period. On Day 6 in each Period, no meals were served as subjects were released from the clinic after the completion of all clinical procedures.

Evaluation of Pharmacokinetics

a. Plasma Collection and Analysis

Blood samples were obtained within 30 minutes prior to dosing (i.e., pre-dose) and 1, 2, 3, 4, 4.5, 5, 6, 7, 8, 9, 10, 11, 12, 14, 16, and 24 hours after dosing in each period. Samples were drawn in the testing area with subjects seated upright in a chair. Blood was collected into 7-mL vacutainers containing sodium heparin and was allowed to cool in an ice chip and water bath for at least 5 minutes after collection. Samples were centrifuged at 4° C. at approximately 3000 rpm for 15 minutes to separate the plasma. The plasma fraction was transferred into two chilled, pre-labeled polypropylene tubes. Samples were stored frozen at approximately −70° C. until analysis.

Bioanalysis of plasma niacin and NUA concentrations were carried out by HPLC chromatography with MS/MS detection. Niacin and NUA concentrations were obtained from the same injection. The lower limit of quantitation (LLQ) for both niacin and NUA were 2 ng/mL in plasma. Quality control samples were evaluated with each analytical run.

b. Urine Collection and Analysis

Urine will was collected in the following intervals: −24 to −18, −18 to −12, −12 to −6, −6 to 0 hours (i.e., prior to dosing) and 0 to 6, 6 to 12, 12 to 18, 18 to 24, 24 to 48, 48 to 72 and 72 to 96 hours after dosing (for a total of 11 collections/treatment)

Urine was collected and transferred into plastic containers equipped with tightly fitting lids. Collected urine was kept refrigerated or in an ice-water bath during the collection interval. The collection containers were labeled to identify the subject number and initials, collection interval, and protocol number. The total weight of urine collected during each interval, measured to the nearest tenth of a gram (e.g. 100.1 g) was recorded. The start and stop date(s) and times of each urine collection interval were also recorded. Two aliquots (approximately 2.5 mL) from each collection interval were transferred into two appropriately labeled polypropylene tubes. The specimens for analysis were labeled to identify Kos, protocol number, subject number, date, collection interval, study day, and period. The aliquot was stored at approximately −20° C. until ready for shipment. An additional aliquot was obtained for specific gravity determination by the clinical site.

Urine samples were analyzed for niacin, NUA, MNA and 2-PY. Bioanalysis of urine niacin, NUA, MNA and 2-PY concentrations were carried out by HPLC chromatography with MS/MS detection. Niacin and NUA concentrations were obtained from the same injection. In urine the LLQ values were 20 ng/mL for niacin and 200 ng/mL for NUA. MNA and 2-PY had LLQ values of 500 ng/mL and 2500 ng/mL respectively. Quality control samples were evaluated with each analytical run.

c. Plasma Pharmacokinetic Parameters and Urinary Recovery

Data from subjects providing sufficient information to calculate pharmacokinetic parameters for at least one treatment was included in the pharmacokinetic analysis. The following pharmacokinetic parameters were calculated for each subject after administration of each treatment:

From plasma niacin and NUA data:
Cₘₐₓ: the maximum plasma concentration observed
Tₘₐₓ: the time of the maximum observed concentration
AUC₀₋ₗₐₛₜ: the area under the plasma concentration-time profile; calculated from time 0 to the last measurable concentration by the linear trapezoidal rule

Additional PK parameters were also calculated from the NUA data:

AUC_{0-inf}: the area under the plasma concentration-time profile; calculated from time 0 to infinity as AUC₀₋ₗₐₛₜ+Cₜ/Kₑₗ where, Cₜ is the last observed quantifiable concentration and Kₑₗ is the terminal elimination rate constant.
T_{1/2}: terminal elimination half-life calculated as 0.693/Kₑₗ

From niacin, NUA, MNA, and 2PY data in urine:
CumXᵤ: urine recovery for each analyte individually, (i.e., the amount of each analyte recovered in urine)
Fe: fraction excreted in the urine calculated as

$$\% Fe = \frac{CumXu}{Dose} ⨯ \frac{MW\_of \_Niacin}{MW\_of \_Analyte} ⨯ 100$$
Total % Fe: total recovery of niacin, NUA, MNA, and 2PY

Concentrations below the LLQ were treated as zero, and actual sample collection times were used in analysis. Individual plots of plasma niacin and NUA concentrations were generated using WinNonlin Professional Network Edition, Version 4.1. Mean plots of plasma niacin and NUA concentrations and urine recovery data were generated by WinNonlin 4.1 and Microsoft® Excel 2000. Plasma pharmacokinetic parameters were determined from each profile by WinNonlin. Terminal phase slope and apparent half-life were not calculated for plasma niacin data due to the small number of quantifiable niacin concentrations in each plasma profile and lack of clearly defined terminal phase.

All urinary pharmacokinetic parameters were determined using WinNonlin 4.1 and Excel 2000. The amount of each analyte recovered in the urine was determined by multiplying the analyte concentration by the volume of urine collected for each interval; the amount recovered in urine for the 24-hour intervals after dosing was then corrected for baseline recovery by subtracting the amount found in the 24-hour pre-dose interval.

The molecular weights of the analytes are: niacin, 123.1; NUA, 180.2; MNA, 137.1; 2PY, 153.1. Percent recovery of the individual analytes was summed to calculate the total percent of the dose recovered.

Statistical Analysis

Demographics were summarized using the SAS® System for Windows™, version 8.02. Continuous demographic data were summarized by mean, standard deviation (SD), median, minimum, and maximum values.

Bioequivalence parameters were evaluated by using the bioequivalence wizard built in WinNonlin 4.1 using the natural log-transformed data. The model included sequence, subjects within sequence, period, and treatment.

Bioequivalence was assessed by classical 90% confidence interval (CI) estimates for the ratio of the test to reference (ERN-1 NSP, ERN-2/NSP, ERN-3/NSP) of least square means, based on natural-log transformed data. Treatments were considered to be bioequivalent if the 90% CIs were within 80 to 125%. For bioequivalence determinations the parameters used were Cₘₐₓ for NUA in plasma and total amount of niacin and metabolites excreted in the urine. Confidence intervals were also determined for niacin plasma (Cₘₐₓ and AUC₀₋ₗₐₛₜ) and individual % Fe (Niacin, NUA, MNA, 2PY) for urine data.

Results

Forty-four healthy, nonsmoking women and men, 40 to 70 years-of-age, inclusive, who met the protocol inclusion and exclusion criteria, were enrolled in the study. Subjects were selected based on no tobacco use for at least 120 days prior to receiving the first dose of study medication and the absence of any clinically significant findings from the medical history, physical examination, electrocardiogram (ECG), and clinical laboratory evaluations.

Of the 44 subjects who were enrolled, 41 subjects completed the study. Twenty-eight subjects were men, and 16 subjects were women. The mean age was 51 years, the mean weight was 171 pounds, and the mean height was 68 inches. Thirty-six subjects were Caucasian, 6 were Black, and 2 were Hispanic. Detailed demographics are illustrated below in Table 18.

TABLE 18
Summary of Subject Demographics

| Parameter | Category | Statistic | All Subjects | Male | Female |
|---|---|---|---|---|---|
| Number of Subjects | | | 44 | 28 | 16 |
| Age | | Mean | 50.7 | 48.1 | 55.4 |
| | | SE | 1.11 | 1.25 | 1.60 |
| | | Median | 50 | 46 | 56 |
| | | Min, Max | 40, 67 | 40, 67 | 44, 67 |
| Gender | Male | n (%) | 28 (63.6%) | — | — |
| | Female | n (%) | 16 (36.4%) | — | — |
| Race/Ethnicity | Caucasian | n (%) | 36 (81.8%) | 23 (52.3%) | 13 (29.5%) |
| | Black | n (%) | 6 (13.6%) | 3 (6.8%) | 3 (6.8%) |
| | Hispanic | n (%) | 2 (4.5%) | 2 (4.5%) | 0 |
| Height (in) | | Mean | 68.3 | 70.3 | 64.9 |
| | | SE | 0.63 | 0.71 | 0.52 |
| | | Median | 68 | 71 | 65 |
| | | Min, Max | 61, 76 | 61, 76 | 62, 70 |
| Weight (lbs) | | Mean | 170.5 | 179.7 | 154.5 |
| | | SE | 3.36 | 3.26 | 5.35 |
| | | Median | 169 | 178 | 146 |
| | | Min, Max | 134, 206 | 155, 206 | 134, 204 |
| Frame Size | Small | n (%) | 6 (13.6%) | 5 (11.4%) | 1 (2.3%) |
| | Medium | n (%) | 31 (70.5%) | 21 (47.7%) | 10 (22.7%) |
| | Large | n (%) | 7 (15.9%) | 2 (4.5%) | 5 (11.4%) |
| Elbow Breadth (in) | | Mean | 2.7 | 2.8 | 2.5 |
| | | SE | 0.04 | 0.04 | 0.06 |
| | | Median | 3 | 3 | 3 |
| | | Min, Max | 2.13, 3.25 | 2.25, 3.25 | 2.13, 3.00 |

a. Assessment of Bioequivalence

Forty-three subjects provided plasma and urine data for the reference (NSP) treatment. Forty-two subjects provided plasma and urine data for the ERN-1 and ERN-2 test treatment. Forty-one subjects provided plasma and urine data for the ERN-3 test treatment. Nominal times were used for mean tables, mean plots, individual plots and concentration listings. For PK analyses the following rules were used:

For sampling times from 1-10 hours (inclusive): Actual times were used for deviations of 5 minutes or greater. For deviations less than 5 minutes, nominal times will be used.

For sampling times greater than 10 hours: Actual times were used for deviations of 10 minutes or greater. For deviations less than 10 minutes, nominal times will be used.

Summary statistics for plasma niacin and NUA pharmacokinetic parameters are shown in Table 18A.

TABLE 18a
Summary of Plasma Bioavailability Parameters and Statistics

| | Plasma Niacin | | | | | | Plasma NUA | | | | | |
|---|---|---|---|---|---|---|---|---|---|---|---|---|
| | | | | Statistics | | | | | | Statistics | | |
| | | | | | 90% | 90% | | | | | 90% | 90% |
| | | | % | | CI | CI | | | % | | CI | CI |
| Parameter | Mean | SD | CV | Ratio (%)^{a} | Lower | Upper | Mean | SD | CV | Ratio (%)^{a} | Lower | Upper |
| Cₘₐₓ (ng/mL) | | | | | | | | | | | | |
| ERN-1 | 5288.2 | 4848.3 | 92 | 138.88 | 113.29 | 170.26 | 2821.7 | 1429.9 | 51 | 110.50 | 101.10 | 120.78 |
| ERN-2 | 4223.2 | 3736.3 | 88 | 115.86 | 94.39 | 142.22 | 2616.0 | 1265.7 | 48 | 105.06 | 96.06 | 114.90 |
| ERN-3 | 5670.7 | 4295.6 | 76 | 165.68 | 134.89 | 203.48 | 3057.5 | 1474.3 | 48 | 123.09 | 112.52 | 134.65 |
| NSP | 4706.5 | 5882.7 | 125 | — | — | — | 2540.0 | 1374.2 | 54 | — | — | — |
| AUC₀₋ₗₐₛₜ | | | | | | | | | | | | |
| (ng * hr/mL) | | | | | | | | | | | | |
| ERN-1 | 13896.3 | 15737.1 | 113 | 136.84 | 114.32 | 163.80 | 13663.5 | 7651.5 | 56 | 103.70 | 96.46 | 111.48 |
| ERN-2 | 10207.0 | 11548.3 | 113 | 112.17 | 93.59 | 134.44 | 12068.6 | 6458.2 | 54 | 94.72 | 88.06 | 101.89 |
| ERN-3 | 13507.0 | 14409.4 | 107 | 150.68 | 125.67 | 180.67 | 13960.2 | 7411.3 | 53 | 109.27 | 101.57 | 117.55 |
| NSP | 12314.9 | 21077.0 | 171 | — | — | — | 13069.5 | 7599.8 | 58 | — | — | — |
| Tₘₐₓ (hr)^{b} | | | | | | | | | | | | |

| | | |
|---|---|---|
| ERN-1 | 6.00 (1.00-9.08) | 6.00 (2.00-9.08) |
| ERN-2 | 5.00 (2.00-8.00) | 5.50 (2.00-9.00) |
| ERN-3 | 6.00 (1.00-8.00) | 6.00 (1.00-8.00) |
| NSP | 5.00 (2.00-8.00) | 5.00 (2.00-9.00) |

Each treatment consists of 2000 mg niacin, N = 42 for ERN-1 and ERN-2, 41 for ERN-3, and 43 for NSP
NSP is the reference treatment
^{a}Ratio of the least square means of the natural-log transformed Niacin and NUA Cmax and AUC₀₋ₗₐₛₜ.
^{b}Median and range are presented for Tₘₐₓ

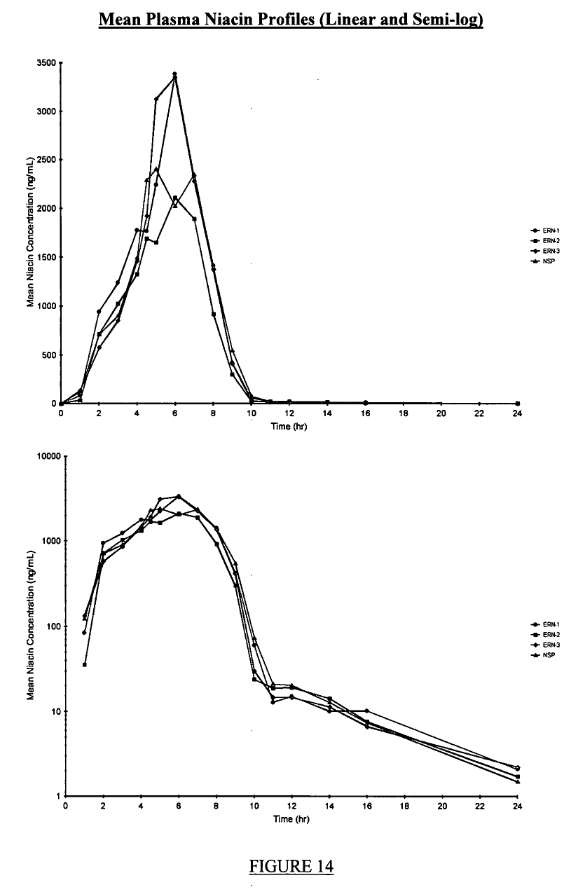

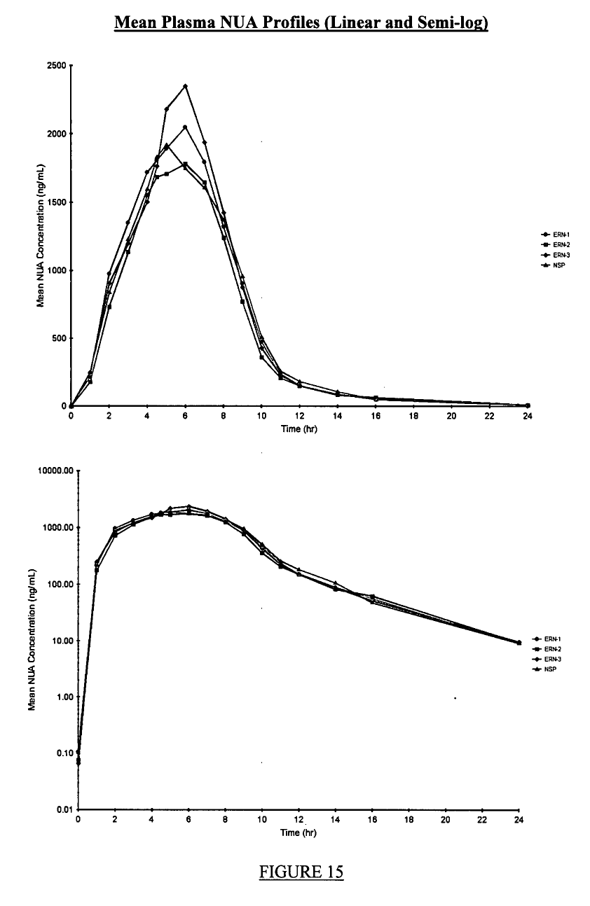

Mean plasma profiles for niacin and NUA are shown in FIGS. 14 and 15.

b. Plasma Data

Plasma Niacin

All subjects had a pre-dose values below the LLQ. All subjects had measurable niacin concentrations from 4.5 to 12 hours post dose after each treatment.

Mean niacin Cₘₐₓ was 5288, 4223, 5671 and 4707 ng/mL for ERN-1, ERN-2, ERN-3 and NSP, respectively. Mean niacin AUC₀₋ₗₐₛₜ was 13896, 10207, 13507 and 12315 ng*hr/mL for ERN-1, ERN-2, ERN-3 and NSP, respectively. Median Tₘₐₓ for niacin was 6.0 hours for ERN-1 and ERN-3, and 5 hours for ERN-2 and NSP.

The ratios for natural-log transformed Cₘₐₓ and AUC₀₋ₗₐₛₜ were greater than 100% for all three-test treatments when compared to NSP. The ratios for niacin Cₘₐₓ were 139%, 116% and 166% for ERN-1, ERN-2, and ERN-3, respectively. The ratios for niacin AUC_{0-last were} 137%, 112% and 151% for ERN-1, ERN-2, and ERN-3, respectively. The 90% CIs for natural-log transformed niacin Cₘₐₓ were 113 to 170%, 94 to 142% and 135 to 203% for ERN-1, ERN-2 and ERN-3, respectively. For natural-log transformed niacin AUC₀₋ₗₐₛₜ the 90% CIs were 114 to 164%, 94 to 134%, and 126 to 181% for ERN-1, ERN-2 and ERN-3, respectively. The 90% CIs for both the Cₘₐₓ and AUC₀₋ₗₐₛₜ were outside the equivalence range of 80-125%.

The niacin data was highly variable with CVs ranging from 76 to 171% for Cₘₐₓ and AUC₀₋ₗₐₛₜ for all four treatments.

Plasma Nicotinuric Acid

Three subjects had positive pre-dose NUA concentrations. These were subject 0028 (Period 2, ERN-1, concentration 4.47 ng/mL), subject 30 (Period 2, ERN-3, concentration 2.75 ng/mL), and subject 33 (Period 2, ERN-2, concentration 3.26 ng/mL). No correction was made to these plasma profiles since the pre-dose concentrations were only about 0.24%, 0.06% and 0.53% of the Cₘₐₓ for subjects 0028, 0030 and 0033 respectively. All subjects had measurable NUA concentrations from 3 to 16 hours post dose after each treatment.

Mean NUA Cₘₐₓ was 2822, 2616, 3058 and 2540 ng/mL for ERN-1, ERN-2, ERN-3 and NSP, respectively. Mean NUA AUC₀₋ₗₐₛₜ was 13664, 12069, 13960 and 13070 ng*hr/mL for ERN-1, ERN-2, ERN-3 and NSP, respectively. Median NUA Tₘₐₓ was 6.0 hours for ERN-1 and ERN-3, 5.5 hours for ERN-2 and 5.0 hours for NSP.

The terminal elimination rate was calculated for each subject and treatment when possible. Mean t_{1/2} was 3.4 hr for ERN-1, ERN-2 and ERN-3 respectively and 3.1 hr for NSP. Mean AUC_{inf} was 13602, 11913, 14136 and 13009 ng*hr/mL for ERN-1, ERN-2, ERN-3 and NSP, respectively

The ratios for natural-log transformed Cₘₐₓ and AUC₀₋ₗₐₛₜ were greater than 100% for treatments ERN-1 and ERN-3 when compared to NSP. For ERN-2, the Cₘₐₓ ratio was greater than 100% while the AUC₀₋ₗₐₛₜ ratio was less than 100%. The ratios for NUA Cₘₐₓ were 111%, 105% and 123% for ERN-1, ERN-2, and ERN-3, respectively. The ratios for NUA AUC₀₋ₗₐₛₜ were 104, 95% and 109% for ERN-1, ERN-2, and ERN-3, respectively. The 90% CIs for natural-log transformed NUA Cₘₐₓ were 101 to 121%, 96 to 115% and 113 to 135% for ERN-1, ERN-2 and ERN-3 respectively. For natural-log transformed NUA AUC₀₋ₗₐₛₜ the 90% CIs were 96 to 111%, 88 to 102% and 102 to 118% for ERN-1, ERN-2 and ERN-3 respectively. The 90% CIs for both the Cₘₐₓ and AUC₀₋ₗₐₛₜ were within the bioequivalence range of 80-125% for ERN-1 and ERN-2. For ERN-3, the 90% CI for Cₘₐₓ was outside the 80-125% range but that for AUC₀₋ₗₐₛₜ was within the 80-125% range.

The NUA data was highly variable with CVs about 48-58% for Cₘₐₓ and AUC₀₋ₗₐₛₜ for all four treatments.

c. Urinary Recovery of Niacin and Metabolites

A specific gravity of 1 was used to convert urine weights to volumes. This was based on a previous study with NIASPAN® where the mean specific gravity measured in 962 samples was 1.009 g/mL and the maximum specific gravity measured in 962 samples was 1.025 g/mL.

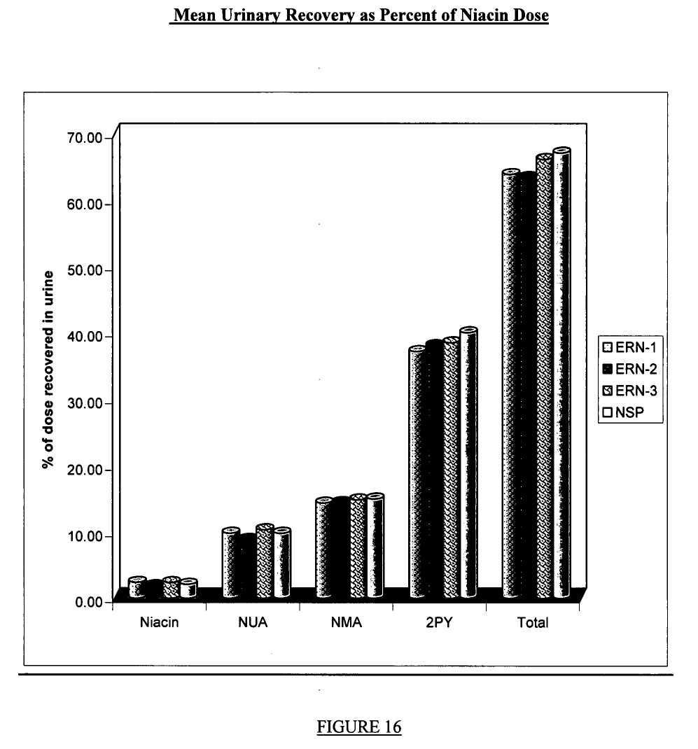

Mean urine recovery data are shown in Table 19 and depicted in FIG. 16.

TABLE 19
Summary of Urinary Bioavailability Parameters and Statistics

| | | Urine Recovery | | | Statistics | | |
|---|---|---|---|---|---|---|---|
| | | (% of dose) | | | | Lower 90% | Upper 90% |
| | | Mean | SD | % CV | Ratio (%)^{a} | CI | CI |
| Niacin | ERN-1 | 2.41 | 2.08 | 86.3 | 134.91 | 111.24 | 163.60 |
| Recovery | ERN-2 | 1.91 | 1.70 | 89.1 | 112.07 | 92.30 | 136.07 |
| | ERN-3 | 2.37 | 1.74 | 73.6 | 147.01 | 121.01 | 178.60 |
| | NSP | 2.11 | 2.73 | 129.3 | — | — | — |
| NUA | ERN-1 | 9.90 | 4.39 | 44.4 | 100.17 | 92.03 | 109.02^{c} |
| Recovery | ERN-2 | 8.96 | 4.16 | 46.4 | 92.01 | 84.49 | 100.19^{c} |
| | ERN-3 | 10.41 | 4.81 | 46.2 | 105.99 | 97.31 | 115.45^{c} |
| | NSP | 9.88 | 4.52 | 45.7 | — | — | — |
| MNA | ERN-1 | 14.42 | 4.05 | 28.1 | 94.22 | 89.04 | 99.69^{c} |
| Recovery | ERN-2 | 14.52 | 4.51 | 31.1 | 93.26 | 88.10 | 98.71^{c} |
| | ERN-3 | 14.90 | 5.18 | 34.8 | 96.35 | 91.01 | 102.01^{c} |
| | NSP | 15.05 | 3.50 | 23.3 | — | — | — |
| 2PY | ERN-1 | 37.17 | 6.41 | 17.2 | 93.16 | 88.90 | 97.64^{c} |
| Recovery | ERN-2 | 38.06 | 5.95 | 15.6 | 94.89 | 90.52 | 99.47^{c} |
| | ERN-3 | 38.49 | 7.91 | 20.5 | 94.91 | 90.52 | 99.50^{c} |
| | NSP | 40.01 | 7.84 | 19.6 | — | — | — |
| Total | ERN1 | 63.91 | 9.34 | 14.6 | 94.79 | 90.85 | 98.90^{c} |
| Recovery^{b} | ERN2 | 63.44 | 9.36 | 14.8 | 94.28 | 90.35 | 98.39^{c} |
| | ERN3 | 66.16 | 12.12 | 18.3 | 97.70 | 93.60 | 101.97^{c} |
| | NSP | 67.14 | 9.47 | 14.1 | — | — | — |

Each treatment consists of 2000 mg niacin, N = 42 for ERN-1, ERN-2 and NSP, and 41 for ERN-3.
NSP is the reference treatment
^{a}Ratio of the least square means of the natural-log transformed Recovery of Niacin, NUA, MNA, 2Py, and Total Recovery.
^{b}Recovery of niacin, NUA, MNA, and 2PY combined.
^{c}Suggests bioequivalence (i.e., 90% CI within 80-125% for natural-log transformed MNA, 2PY Recovery, and Total Recovery).

Total Recovery

Total recovery of niacin in the urine as niacin, NUA, MNA, and 2PY was 67.14% for NSP and 63.91%, 63.44% and 66.16% for ERN-1, ERN-2, and ERN-3, respectively. The least square means ratio of the logₑ transformed % Fe for total recovery were 95%, 94% and 98% respectively for ERN-1, ERN-2 and ERN-3. The 90% CI for the ratios were 91 to 99%, 90 to 98% and 94 to 102%, respectively for ERN-1, ERN-2 and ERN-3 indicating that the total amount excreted in urine by the 3 test formulations were equivalent to NSP based on the 80-125% confidence interval.

d. Conclusions of the Bioequivalent Assessment

Pharmacokinetic analysis of NUA data indicated that peak exposure measured by Cₘₐₓ was higher for all the 3 test formulations (ERN-1, ERN-2, ERN-3) as compared to the test formulation NSP by 5 to 23%. The 90% CI for the least square mean ratios for the logₑ transformed Cₘₐₓ were within the 80-125% range for ERN-1 and ERN-2 indicating that these formulations were bioequivalent to NSP with respect to NUA. For ERN-3, the 90% CI were outside the 80-125% range for Cₘₐₓ, indicating that formulation ERN-3 was not bioequivalent to NSP.

The mean total amount of niacin and metabolites excreted in urine was 63 to 66% for the 3 test formulations and 67% for NSP. Fraction excreted was smallest for the parent niacin followed by NUA, MNA and 2PY (37.2-40.0%). Total recovery measured was 2 to 6% lower for the test formulations as compared to NSP. The 90% CI for the least square mean ratios of the logₑ transformed total recovery was within the 80-125% range, and thus equivalent for the 3 test formulations as compared to NSP.

Accordingly, one embodiment of the invention comprises a reformulated 1000 mg extended-release niacin pharmaceutical composition which when administered to subjects in a bioequivalence study comparing a single dose of four 500 mg NIASPAN® tablets to a single dose of to of said reformulated 1000 mg extended-release niacin compositions provides 90% CI's for a natural-log transformed ratio of the appropriate bioavailability parameters within a 80% to 125% interval.

In a preferred embodiment, the bioavailability parameters are NUA Cmax (ng/ml) and Total Recovery, or Niacin Cmax (ng/ml) and Niacin AUC.

### EXAMPLE 6

The purpose of this study was to determine the bioequivalence (BE) of the coated versus uncoated, 1000 mg extended-release niacin tablets of the invention (referred to hereinafter as “reformulated” tablets), when administered as a single dose of 2000 mg.

Study Design

The study was a randomized, single-center, open-label, single-dose, two-way crossover study in 44 healthy, nonsmoking male and female volunteer subjects, 40 to 70 years-of-age, inclusive. Drop-outs were not replaced. Each subject received two niacin formulations, Test and REF, in the same single dose of 2000 mg on two separate occasions, with a washout period of 10 days between doses. The Test consisted of two tablets of coated reformulated 1000 mg extended-release niacin and the REF consisted of two tablets of uncoated reformulated 1000 mg extended-release niacin. Each dose was administered with 240 mL of water after a low-fat snack beginning at approximately 22:00 hours (hrs) on Day 1 of each period. Subjects were housed during the 6-day study period (day-1 to day 5) of each treatment and received meals according to sponsor-provided menus during each treatment period. No other medications, vitamins, herbal or nutritional supplements were permitted during the study.

Serial blood samples were collected from within 30 min prior to dosing out through 24 hrs post dose after dosing at the intervals: −30 min (pre-dosing), 1, 2, 3, 4, 4.5, 5, 6, 7, 8, 10, 12, 14, 16, and 24 hrs (post-dosing). Urine was collected from 24 hrs prior to dosing until 96 hrs after dosing at the intervals: −24 to −18, −18 to −12, −12 to −6, and −6 to 0 hrs (pre-dosing); 0 to 6, 6 to 12, 12 to 18, 18 to 24, 24 to 48, 48 to 72, and 72 to 96 hrs (post-dosing). Plasma was analyzed for niacin, and NUA. Urine was analyzed for niacin, and its metabolites: NUA, MNA, and 2-PY.

Subjects began meals at the same times of each day when they were confined to the clinic during each period. Meals were held at the same for each period, and the entire contents of each meal were required to be consumed. Breakfast, lunch, dinner, and an evening snack began at approximately 07:00, 12:00, 17:00, and 21:45, respectively. The actual meal or snack time for each subject was scheduled relative to the actual dosing time. Subjects were required to drink a minimum of 720 mL of water on Day −1 and 1440 mL of water on Day 1 through 5 in addition to the 240 mL of water given with the study medication on Day 1.

On Day −1, dinner and an evening snack were served. On Days 1 through 5, breakfast, lunch, dinner, and an evening snack were served. The evening snack was consumed within 15 minutes just prior to dosing on Day 1 in each period. On Day 6 in Period 2, no meals were served as subjects were discharged from the clinic after the completion of all clinical procedures.

Evaluation of Pharmacokinetics

a. Plasma Collection and Analysis

Serial blood samples were collected within 30 min prior to dosing through 24 hrs after dosing in each period (15 samples/treatment). Each blood sample was collected into one 17-mL vacutainer containing sodium heparin and was allowed to cool in an ice-chip and water bath for a minimum of 5 min after collection. Samples were centrifuged at 4° C. at approximately 3000 rpm for 15 min to separate the plasma. Each plasma sample was divided into two aliquots, Aliquot A and B, and transferred into two pre-chilled, appropriately labeled polypropylene tubes Samples were then stored frozen at approximately −20° C. until analysis.

Niacin and NUA concentrations were analyzed by validated liquid chromatography tandem mass spectroscopy (LC/MS/MS). Niacin and NUA concentrations were obtained from the same injection. The lower limit of quantitation (LLQ) for both niacin and NUA was 2 ng/mL in plasma. Quality control samples were evaluated with each analytical run.

b. Urine Collection and Analysis

Urine was collected for the following intervals: −24 to −18, −18 to −12, −12 to −6, −6 to 0 hrs (prior to dosing), and 0 to 6, 6 to 12, 12 to 18, 18 to 24, 24 to 48, 48 to 72, 72 to 96 hrs after dosing (for a total of 11 collections).

Urine was collected and transferred into plastic containers equipped with tightly fitting lids. Collected urine was kept refrigerated or in an ice-water bath during the collection interval. The collection containers were labeled to identify the subject number and initials, collection interval, and protocol number. The empty containers were weighed to the nearest tenth of a gram (e.g., 100.1 g) and this was written on the container and documented on the lab's source document worksheets. At the end of each interval, the total weight of the container and the collected urine was measured to the nearest tenth of a gram and recorded. The weight of the urine was derived by subtracting the weight of the empty container from the total weight of the container plus the urine. In some cases, the volume of urine during a given collection interval exceeded the capacity of a single container; therefore a second container was required to obtain a complete urine collection. The start and stop date(s) and times of each urine collection interval were also recorded. Two aliquots (approximately 2.5 mL each) from each collection interval were transferred into two appropriately labeled polypropylene tubes. If more than one container was required during a particular collection interval, the urine from both containers was mixed together before the aliquots were taken. Samples were stored frozen at approximately −20° C. until analysis.

Urine samples were analyzed for concentrations of niacin, NUA, MNA and 2-PY by validated LC/MS/MS. Urine niacin and NUA concentrations were obtained from the same injection while MNA and 2-PY concentrations were obtained from the same injection. In urine the LLQ values were 20 ng/mL for niacin and 200 ng/mL for NUA. MNA and 2PY had LLQ values of 500 ng/mL and 2500 ng/mL, respectively. Quality control samples were evaluated with each analytical run.

c. Plasma Pharmacokinetic Parameters and Urinary Recovery

Data from subjects providing sufficient information to calculate PK parameters for at least one treatment were included in the PK analysis. For niacin and NUA in plasma, the following PK parameters were calculated for each subject following administration of each treatment:
Cₘₐₓ: the maximum concentration observed
Tax: the time of the maximum observed concentration
AUCₗₐₛₜ: the area under the concentration-time profile from time 0 to the last measurable (non-zero) concentration by the linear trapezoidal rule
AUC_{inf}: the area under the plasma concentration-time profile from time 0 to infinity;
calculated as the sum of AUCₗₐₛₜ and Cₜ over λ_{z} where Cₜ is the last observed concentration and λ_{z} is the terminal elimination rate constant obtained from the plot of natural-log concentration versus time plots
T_{1/2}: the apparent terminal half-life; calculated as a ratio of 0.693 over the λ_{z}
From the urine data of niacin and its metabolites (NUA, MNA, and 2-PY) the following parameters were computed:
CumXᵤ: cumulative amount of each metabolite recovered from urine from 0 to 96 hrs after dosing
% Fe: fraction of each metabolite excreted in the urine relative to dose of niacin after correction for baseline recovery and molecular weight in 96 hrs after dosing
Total % Fe: total fraction of the four metabolites in 96 hrs after dosing
The % Fe, for each analyte in urine calculated as

$$\% Fe = \frac{CumXu}{Dose} ⨯ \frac{MW\_of \_Niacin}{MW\_of \_Analyte} ⨯ 100$$

Concentrations below the limit of quantitation were treated as zero. The amount of niacin and its metabolites recovered in the urine was determined by multiplying each metabolite concentration by the volume of urine collected for each interval. The total amount recovered in urine for each 24-hour interval after dosing was adjusted for baseline by subtracting the amount recovered in the 24-hour pre-dose interval. If any post-dosing measurement was less than baseline the amount was set to zero. The molecular weights of niacin and its metabolites are 123.1, 180.2, 137.1, and 153.1 for niacin, NUA, MNA, and 2-PY, respectively. The sum of % Fe from the four urine analytes, was calculated and designated as total % Fe.

Bioavailability parameters (as described above) were calculated using WinNonlin Linear Mixed Effects Modeling/bioequivalence, Version 5.0.1 (Jul. 26, 2005).

Statistical Analysis

Statistical analyses of the bioavailability parameters calculated above were performed using an SAS® System for Windows™, version 8.2, was used for data analysis.

Plasma pharmacokinetic parameters (Cₘₐₓ, Tₘₐₓ, T_{1/2}, AUCₗₐₛₜ and AUC_{inf}), their natural log-transformed value (except for Tₘₐₓ, and T_{1/2}), and summary statistics (m, mean, std, median, min, max, CV %) were calculated by treatment and period. Plasma concentrations of niacin and NUA are summarized by time and treatment.

For the niacin and NUA PK analysis, it is assumed that the data of the natural log-transformed Cₘₐₓ and AUCₗₐₛₜ follow a normal distribution and are independent between the two treatments. The data were fitted to an ANOVA model with mixed effects using SAS PROC MIXED with treatment, period, and sequence as fixed effects and subject within sequence as a random effect. The Test/REF ratios of Cₘₐₓ and AUCₗₐₛₜ and their corresponding 90% confidence intervals were estimated based on this model.

The mean recovery of niacin and its metabolites from urine was calculated and summarized by treatment and by interval. The CumXᵤ and % Fe of individual components and the total in 96 hrs after dosing were calculated and summarized by treatment.

The 90% confidence intervals (CIs) for the Test/REF mean ratios of total % Fe were calculated by fitting the same ANOVA model as used for plasma PK analysis.

Subjects' demographic variables (age, gender, race, weight, height, frame size, elbow breadth, and BMI) were summarized by gender. The mean, standard deviation (SD), median, minimum, and maximum of the continuous demographic variables were computed.

Results

Subject disposition is summarized in Table 20 A total of 44 subjects were enrolled in the study after they met the protocol inclusion and exclusion criteria. All the subjects received at least one dose of study medication, and 42 of them completed the study. Forty-four subjects received study medication in Period 1 according to the randomized treatment assignment in the protocol, whereas 42 subjects received study medication in period 2. A total of 2 subjects discontinued from the study. The numbers of subjects who discontinued from the study were in the range of pre-allowed 10% dropout, and were not considered to affect the results or conclusions of the study.

TABLE 20
Summary of Subject Disposition

| Subject numbers | (N) | Percent (%) |
|---|---|---|
| Enrolled | 44 | 100 |
| Completed study | 42 | 95.5 |
| Received at least one dose | 44 | 100 |
| Received medication at Period 1 | 44 | 100 |
| Received medication at Period 2 | 42 | 95.5 |
| Discontinuation | 2 | 4.5 |

Of the enrolled 44 subjects, 20 subjects were men and 24 were women. The mean age was 53.1 years; the mean weight was 161.5 pounds; the mean height was 65.6 inches; the mean elbow breadth was 2.7 inches; the mean BMI was 26.3 kg/m². Frame size was graded as small, medium and large. Nine subjects had a small frame size, 20 subjects had medium and 15 subjects had large frame sizes. Thirty-eight of the subjects were hispanic, 4 were caucasian, and 2 were black. The detailed demographics are summarized in Table 21.

TABLE 21
Summary of Subject Demographics

| | | | | By Gender | |
|---|---|---|---|---|---|
| | | Statistic | All Subjects | Males | Females |
| Number of Subjects | | | 44 | 20 | 24 |
| Age(yrs) | | Mean | 53.1 | 51.7 | 54.3 |
| | | SE | 7.4 | 9.4 | 5.2 |
| | | Median | 54 | 49 | 55.5 |
| | | Min, Max | 40.0, 70.0 | 40.0, 70.0 | 42.0, 65.0 |
| Gender | Male | N (%) | 20 (45.5) | 20 (100.0) | 0 (0.0) |
| | Female | N (%) | 24 (54.5) | 0 (0.0) | 24 (100.0) |
| Race/Ethnicity | Caucasian | N (%) | 4 (9.1) | 4 (20.0) | 0 (0.0) |
| | Black | N (%) | 2 (4.5) | 1 (5.0) | 1 (4.2) |
| | Hispanic | N (%) | 38 (86.4) | 15 (75.0) | 23 (95.8) |
| | Asian | N (%) | 0 (0.0) | 0 (0.0) | 0 (0.0) |
| | Other | N (%) | 0 (0.0) | 0 (0.0) | 0 (0.0) |
| Height (in) | | Mean | 65.6 | 68.7 | 63.1 |
| | | SE | 3.5 | 2.6 | 1.6 |
| | | Median | 65 | 69 | 63 |
| | | Min, Max | 60.0, 72.0 | 64.0, 72.0 | 60.0, 66.0 |
| Weight (lb) | | Mean | 161.5 | 177.3 | 148.3 |
| | | SE | 20.2 | 15.6 | 12.7 |
| | | Median | 159 | 177 | 144.5 |
| | | Min, Max | 130.0, 210.0 | 155.0, 210.0 | 130.0, 175.0 |
| Frame Size | Small | N (%) | 9 (20.5) | 5 (25.0) | 4 (16.7) |
| | Medium | N (%) | 20 (45.5) | 10 (50.0) | 10 (41.7) |
| | Large | N (%) | 15 (34.1) | 5 (25.0) | 10 (41.7) |
| Elbow breadth (in) | | Mean | 2.7 | 2.9 | 2.5 |
| | | SE | 0.3 | 0.2 | 0.2 |
| | | Median | 2.7 | 2.8 | 2.5 |
| | | Min, Max | 2.2, 3.1 | 2.5, 3.1 | 2.2, 2.8 |
| BMI (kg/m²) | | Mean | 26.3 | 26.5 | 26.2 |
| | | SE | 2.2 | 2.2 | 2.2 |
| | | Median | 26.5 | 26.5 | 26.4 |
| | | Min, Max | 22.2, 30.3 | 22.2, 30.3 | 22.6, 29.9 |

a. Assessment of Bioequivalence

Data from 42 subjects in Test treatment and 44 subjects in REF treatment were analyzed to determine the bioequivalence. Actual times relative to dosing time were used in all analyses.

For urine analyses, a specific gravity of 1 g/mL was used to convert urine weights to volumes. This was based on a previous study with NIASPAN® where the mean specific gravity measured in 962 samples was 1.009 g/mL and the maximum specific gravity measured in 962 samples was 1.025 g/mL.

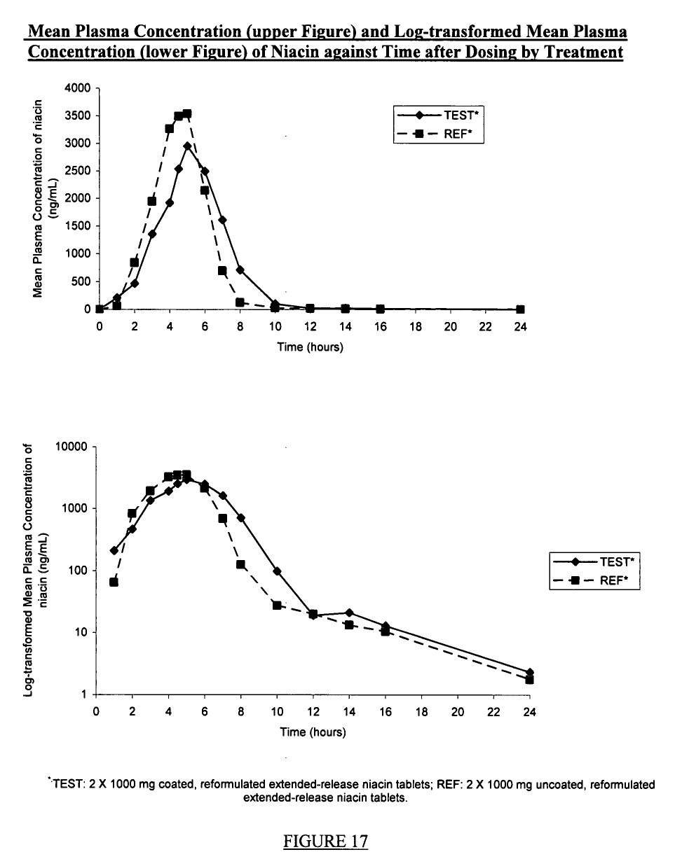

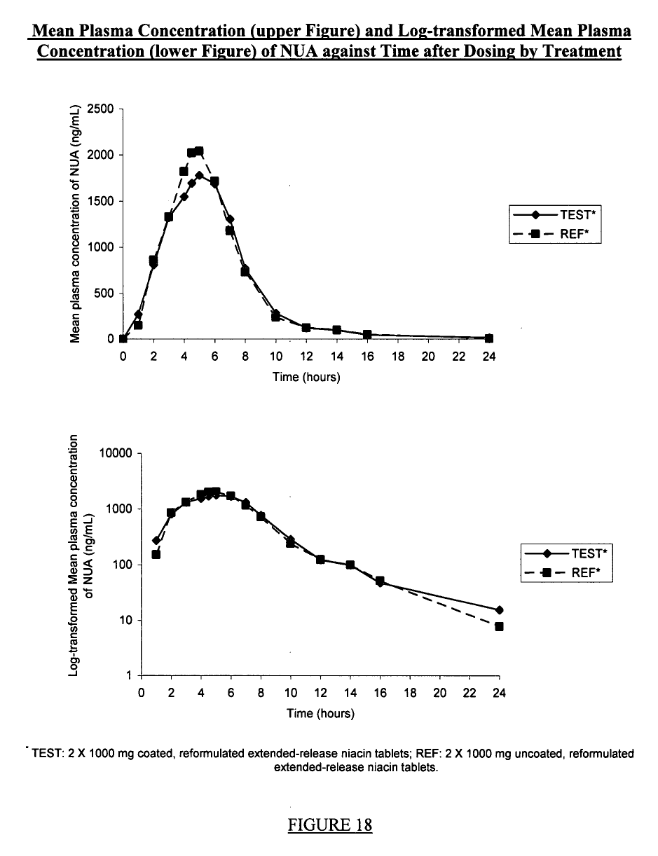

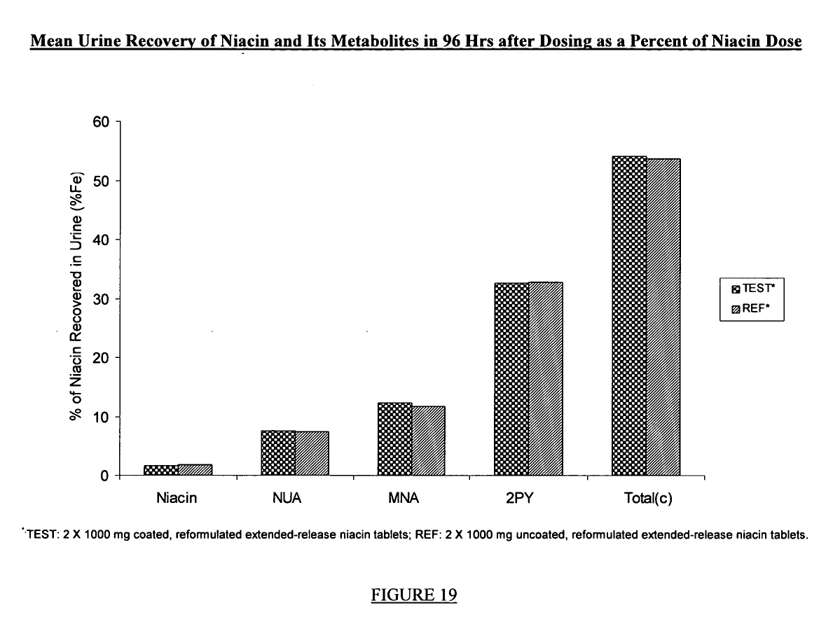

The plots of mean plasma concentrations of niacin and NUA by treatment are shown in FIGS. 17 and 18, respectively. Mean urinary recovery data is shown in FIG. 19.

b. Plasma NUA and Total Amount Excreted in Urine

The primary variables to evaluate Niacin bioequivalence were defined as Cₘₐₓ for NUA and total urinary recovery of niacin and three metabolites (NUA, MNA, and 2PY). Table 22 gives the mean (SD) and statistical results for these two variables. Table 22 also shows the mean (SD) values and statistical analyses for NUA AUCₗₐₛₜ.

TABLE 22
Summary of NUA Plasma Parameters and Total Urinary Recovery

All Subjects
(N_{Test} = 42, N_{REF} = 44)

| | | |
|---|---|---|
| Parameter | Mean (SD) | % Ratio (90% CI) |

NUA Cₘₐₓ^{a} (ng/mL)

| | | |
|---|---|---|
| Test | 2437.0 (1080.71) | 96.21 (89.28, 103.67) |
| REF | 2513.2 (1151.38) | |

Total Recovery^{a,b} (%)

| | | |
|---|---|---|
| Test | 54.04 (13.10) | 101.49 (93.89, 109.69) |
| REF | 53.64 (14.60) | |

NUA AUCₗₐₛₜ^{c} (ng * hr/mL)

| | | |
|---|---|---|
| Test | 11198.9 (6227.64) | 97.87 (89.81, 106.66) |
| REF | 11472.1 (6119.71) | |

^{a}Parameters used to define Niacin bioequivalence
^{b}Recovery of niacin, NUA, MNA, and 2PY combined.
^{c}Supportive data for bioequivalence

As shown in the above table the 90% CI for the natural-log transformed test to reference ratios of the primary BE variables, NUA Cₘₐₓ and total recovery of niacin and metabolites were within 80 to 25%. The test over reference ratios for natural-log transformed NUA AUCₗₐₛₜ was also within 80 to 125%.

The terminal elimination rate was calculated for each subject by treatment. Mean NUA T_{1/2} were 3.16 and 3.04 hrs, mean NUA Tₘₐₓ were 4.90 and 4.80 hrs, and mean NUA AUC_{inf} were 10914.7 and 11770.6 ng*hr/ml, for Test and REF, respectively.

c. Plasma Niacin

Mean PK parameters for plasma niacin along with statistical analyses are given in Table 23 The test over reference ratios for natural-log transformed niacin Cₘₐₓ and AUCₗₐₛₜ were less than 100%. The 90% CI for the ratios of natural-log transformed niacin Cₘₐₓ and AUCₗₐₛₜ, were outside the 80 to 125% interval due to high variability.

TABLE 23
Summary of Niacin Plasma Parameters

All Subjects
(N_{Test} = 42, N_{REF} = 44)

| | | |
|---|---|---|
| Parameter | Mean (SD) | % Ratio (90% CI) |

Niacin Cₘₐₓ (ng/mL)

| | | |
|---|---|---|
| Test | 5052.4 (5209.48) | 94.13 (76.66, 115.58) |
| REF | 5021.2 (5041.08) | |

Niacin AUCₗₐₛₜ (ng * hr/mL)

| | | |
|---|---|---|
| Test | 12444.2 (15616.99) | 91.99 (76.31, 110.88) |
| REF | 12887.8 (15170.37) | |

Mean T_{1/2} of niacin were 4.73 and 2.94 hrs, mean Tₘₐₓ were 4.68 and 4.64 hrs, and mean AUC_{inf} were 11553.1 and 16134.3 ng*hr/ml, for Test and REF, respectively.

d. Urinary Recovery of Individual Analytes

The mean urine recovery of the individual analytes is given in Table 24.

TABLE 24
Summary of Urinary Excretion of Niacin and its Metabolites

All Subjects
(N_{Test} = 42, N_{REF} = 44)

| | Treatment | Mean (SD) |
|---|---|---|
| Niacin Recovery^{a} | Test | 1.59 (1.63) |
| | REF | 1.81 (2.58) |
| NUA Recovery^{a} | Test | 7.59 (4.26) |
| | REF | 7.36 (4.09) |
| MNA Recovery^{a} | Test | 12.23 (4.07) |
| | REF | 11.75 (3.76) |
| 2-PY Recovery^{a} | Test | 32.63 (8.65) |
| | REF | 32.71 (8.64) |

^{a}Recovery as % of niacin dose

Mean urinary recovery was the highest for 2PY followed by MNA, NUA and niacin.

e. Conclusions of the Bioequivalent Assessment

Bioequivalence was evaluated based on the 90% CIs for mean Test/REF ratios of the NUA Cₘₐₓ and urinary recovery of niacin and its metabolites (Total % Fe). The 90% CIs of Test/REF mean ratios of natural-log transformed rate (NUA Cₘₐₓ) and extent (Total % Fe in urine) of niacin absorption were within the required BE range of 80 to 125% and indicate that the Test and REF formulations are bioequivalent. The 90% CI for NUA AUCₗₐₛₜ were also within the 80 to 125% range supporting the BE conclusion.

For niacin Cₘₐₓ and AUCₗₐₛₜ the upper limits of 90% CIs of Test/REF mean ratios for both niacin Cₘₐₓ and AUCₗₐₛₜ fell within the bioequivalence range and the lower limits were both very close to the lower limit of the bioequivalence range, 80%.

### EXAMPLE 7

For the 1000 mg formulations of the invention analyzed in Examples 4, 5 and 6, the average mean for Cₘₐₓ for NUA (ng/ml), total urine recovery (%), Niacin Cₘₐₓ (ng/ml) and Niacin AUC are illustrated in Table 25 below (excluding ERN-3).

TABLE 25
Mean Bioequivalence Variables for 1000 mg Formulations of the Invention

| | | Ex. 5 (n = 44) | | Ex 6 (n = 44) | | |
|---|---|---|---|---|---|---|
| Parameter | Ex. 4 (n = 44) | ERN-1 | ERN-2 | Coated | Uncoated | Average |
| NUA Cmax | 2621.0 | 2821.7 (1430)* | 2616.0 | 2437.0 (1080.7) | 2513.2 | 2601.8 |
| (ng/ml) | | | | | | |
| Total Rec. | 67.7 (8.4) | 63.91 | 63.44 | 54.04 (14.60) | 53.64 | 60.5 |
| (%) | | | | | | |
| Niacin | 5210.3 | 5288.0 (4848) | 4223.0 (3736) | 5052.4 | 5021.2 | 4958.9 |
| Cmax | | | | | | |
| (ng/ml) | | | | | | |
| Niacin AUC | 12637.4 | 13896.0 (15737) | 10207.0 (11548) | 12444.2 | 12887.8 | 12414.5 |

*( ) = standard deviation

Accordingly, one embodiment of the invention comprises a 1000 mg extended-release niacin pharmaceutical composition which when administered to a patient in need thereof as a single dose of two 1000 mg tablets, provides an in vivo plasma profile with a 90% CI for a natural-log transformed ratio within 80% to 125% for at least one of the following bioavailability parameters:

(a) NUA Cₘₐₓ of 2601.8 ng/mL;

(b) total recovery of urinary niacin 60.5%;

(c) niacin Cₘₐₓ of 4958.9 ng/mL; and

(d) niacin AUC of 12414.5 ng/mL.

Table 25a below illustrates the upper and lower limits of selected bioavailability parameters from Table 25 taking into consideration standard error (shown in parentheses). In particular, the lower limit was calculated by identifying the lowest mean from Examples 4, 5 and 6 above for each parameter identified above in Table 25 and then subtracting two standard errors from that mean to generate a lower limit. Standard error was calculated by dividing the standard deviation by the square root of the sample size (For example, 1430/√44=326). Likewise, the upper limit represents the highest mean from example 4, 5 and 6 for each parameter plus two standard errors.

TABLE 25a
Upper and Lower Limits of Selected Bioavailability Parameters

| Parameter | Lower Limit (Std Er) | Upper Limit (Std Er) |
|---|---|---|
| NUA Cmax (ng/ml) | 2111.0 (326) | 3253 (431) |
| Total Rec. (%) | 49.24 (4.4) | 70.23 (2.53) |
| Niacin Cmax (ng/ml) | 3096 (1126) | 6750 (1462) |
| Niacin AUC | 6723 (3484) | 18643 (4747) |

Accordingly, a further embodiment of the invention comprises a 1000 mg extended-release niacin pharmaceutical composition which when administered to a patient in need thereof as a single dose of two 1000 mg tablets, provides an in vivo plasma profile with a 90% CI for a natural-log transformed ratio within a 80% to 125% interval for at least one of the following bioavailability parameters:

(a) NUA Cₘₐₓ of about 2111.0 ng/mL to about 3253 ng/mL;

(b) total recovery of urinary niacin of about 49.24% to about 70.23%;

(c) niacin Cₘₐₓ of about 3096 ng/mL to about 6750 ng/mL; and

(d) niacin AUC of about 6723 ng/mL to about 18643 ng/mL.

### EXAMPLE 8

### Comparative Incidence of Flushing Induced by 2000 mg Dose of Extended-Release Niacin When Pretreated or Co-Administered with Aspirin

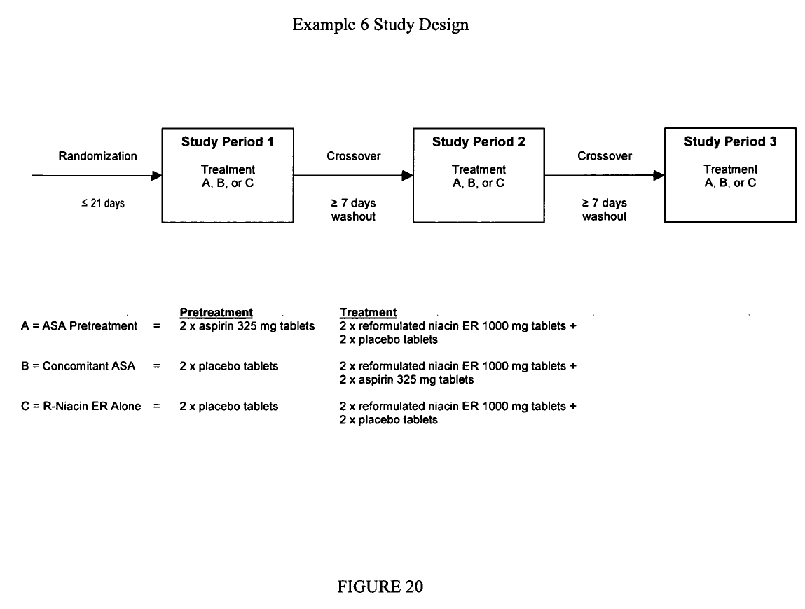

This study was a randomized, double-blind, double-dummy, single-dose, three-way crossover study, conducted at a single center and designed to study the effect of aspirin pretreatment and aspirin co-administration on flushing reactions resulting from oral administration of extended-release niacin tablets of the present invention. The study design and treatments are shown in FIG. 20. Subjects also abstained from using non-study-related aspirin or other NSAIDS at any time during the study. The study was approved by the clinic's Institutional Review Board, and each subject provided written informed consent prior to participation.

The study included healthy adult males 19 to 70 years old with a body mass index (BMI) of 22 to 31 kg/m². Females were excluded from the study to avoid confusing niacin-induced flush events with peri-menopausal flushing. Subjects were confirmed as healthy by a complete physical exam, medical history, electrocardiogram, and results from clinical laboratory testing conducted at the screening visit and at clinic admission for the first study period. Subjects were excluded if they used any tobacco or nicotine product within 4 months of entering the study; had allergy or hypersensitivity to niacin, aspirin, or related derivatives; substance abuse or dependency within the last 3 years; or history of migraine headaches, diabetes, gallbladder disease, liver disease, severe hyper- or hypotension, cardiac abnormality, renal disease, or drug-induced myopathy. Subjects abstained from any prescription medication within 21 days before entering and during the study, and from any over-the-counter medication, vitamin, or herbal preparation within 10 days before entering and during the study.

Screening procedures were completed within 21 days prior to clinic admission for Period 1. For each of the three study periods, subjects remained sequestered for approximately 24 hours, treatments were administered at least 7 days apart, and subjects received meals according to specific menus that controlled niacin and fat content. Meal composition and start time were the same for each study period.

Study Treatments

Study medication was administered orally in a crossover manner according to the randomization schedule. Although the aspirin (ASA) and placebo dosing were different in each study period, the dose of coated, extended-release niacin tablets of the invention (also referred to herein as a “reformulated niacin ER tablet” or “rNER”)—two 1000 mg tablets—were the same. In one period, subjects received two aspirin 325 mg tablets 30 minutes prior to reformulated niacin ER 2000 mg coadministered with two placebo tablets (“ASA Pretreatment”). In another period, subjects received two placebo tablets 30 minutes prior to reformulated niacin ER 2000 mg coadministered with two aspirin 325 mg tablets (“Concomitant ASA”). In a third period, subjects received a control treatment consisting of two placebo tablets 30 minutes prior to reformulated niacin ER 2000 mg coadministered with two placebo tablets (“R-Niacin ER Alone”).

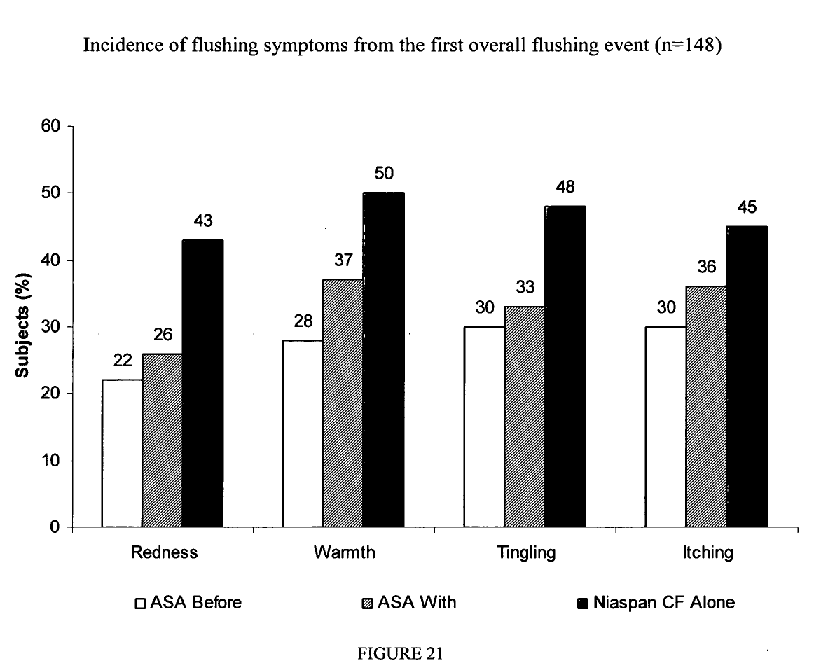

Since evaluating flush events is subjective, study personnel and subjects were blinded by several methods to the identity of the medications administered. In each dosing period, subjects received the same number of tablets for each dose (see FIG. 21). While the placebo and aspirin tablets were similar in appearance, they were not identical; thus, study medication was administered from opaque dosing cups and subjects were blindfolded during study drug administration. The control treatment, R-Niacin ER Alone, was included in the study to assess flushing reactions in the absence of aspirin. Only the study sponsor and the person(s) at the clinical site preparing doses for each period had knowledge of the treatment randomization assignment during the study. Investigators, site personnel, and the study monitor were blinded to the treatment assignment scheme, and any site personnel involved in treatment preparation or administration were prohibited from collecting or assessing flushing events or treatment-emergent adverse events.

Each subject received pretreatment medication and a snack prior to reformulated niacin ER dosing. Subjects received the assigned pretreatment medication (aspirin or placebo) orally with 180 mL of water at approximately 21:30, followed by a low-fat snack starting at approximately 21:45. The snack was consumed in its entirety before the subject received the remainder of the assigned treatment at approximately 22:00 with 240 mL of water. Each medication dose required multiple tablets and was consumed within one minute, as tablets were taken either together at one time or one immediately following another. If needed to swallow tablets, additional water was provided in increments of 120 mL; chewing or biting a tablet was prohibited. Each subject's mouth was inspected after administration of the study dose to verify that the entire dose was consumed.

Flushing Assessments

A flushing event was defined as the subject reporting one or more of the following flushing symptoms: redness, warmth, tingling, and itching; these symptoms could occur individually or concurrently. During each study period, subjects were prompted to assess the presence or absence of flushing symptoms at hourly intervals, for up to 8 hours after reformulated niacin ER administration. Subjects were prompted to record the start time, stop time, and intensity for each flushing symptom in an electronic diary.

Each subject rated their perception of symptom intensity by both continuous and categorical measures. Subjects marked intensity with a vertical line on an electronic horizontal visual analog scale (VAS), anchored from “none” on the left to “intolerable” on the right, and also rated the symptom as mild, moderate, or severe. Symptoms which were easily tolerable and did not limit activities were defined as mild; symptoms causing difficulty in conducting activities were severe.

Each subject similarly rated the first overall flushing event, defined as the first of one or more concurrent flushing symptoms to occur after niacin ER dosing. The start time for the first symptom was also the start time for the first overall flushing event; the overall event ended when the last symptom in that event resolved and at least 30 minutes elapsed without any additional flushing symptom occurring.

Statistical Analysis

A total of 164 subjects were planned for enrollment to assure at least 144 subjects would complete all three treatments. Subjects that discontinued early were not replaced.

All comparisons were conducted as two-tailed with alpha (α)=0.05. The primary endpoint was the number of subjects who experienced at least one flushing event during the study. Flushing incidences were compared between “ASA Pretreatment” and the control treatment, “R-Niacin ER Alone”, using McNemar's test. This test requires subjects to react (in this case, flush) after both treatments to be included in the comparison. Comparisons of flushing incidence were similarly made between the “Concomitant ASA” and “R-Niacin ER Alone” treatments, and between the “ASA Pretreatment” and “Concomitant ASA” treatments.

Secondary endpoints included the number of flushing events, and the intensity, time of onset, and duration of first overall flushing events as well as individual flushing symptoms. The number of events was summarized by frequency count and compared using McNemar's test. VAS intensity assessments were converted from graphic to numerical data by expressing the subject's vertical mark as the distance from the left end of the VAS line (standardized to 100 mm). Intensity measured by VAS and duration were compared between treatments using paired t-tests for means and Wilcoxon signed-rank tests for medians, while intensity measured by categorical scale was compared using Bowker's test of symmetry, a generalization of McNemar's test that also requires subjects to have data for both treatments in the comparison. Comparisons between treatments for secondary endpoints were made for the same treatment pairs as for the primary endpoint.

Adverse events (excluding flushing) were coded using the Medical Dictionary for Regulatory Activities (MedDRA, Version 7.0). Adverse events were not compared between treatments.

Results

A total of 164 men, with mean age 29 years and BMI of 26.5 kg/m², were enrolled and received at least one dose of study medication. Subject demographics are summarized in Table 26. Of the 164 subjects, 148 (90%) received all three treatments and were evaluable for flushing responses. Sixteen subjects (10%) terminated early: 4 (2%) withdrew consent, 4 (2%) were lost to follow-up, 1 (1%) had an adverse event, 3 (2%) had protocol violations, 3 (2%) had a positive drug screen and 1 (1%) was dropped due to dosing error.

TABLE 26
BASELINE SUBJECT DEMOGRAPHICS

| Parameter | | | Subjects |
|---|---|---|---|
| Gender | Male | N (%) | 164 (100%) |
| Race/Ethnicity | Caucasian | N (%) | 140 (85%) |
| | Black | N (%) | 6 (4%) |
| | Hispanic | N (%) | 9 (5%) |
| | Asian | N (%) | 6 (4%) |
| | Other | N (%) | 3 (2%) |
| Age (y) | | Mean (SD) | 29 (12) |
| Height (in) | | Mean (SD) | 71.2 (2.7) |
| Weight (lbs) | | Mean (SD) | 191.6 (21.6) |
| BMI (kg/m²) | | Mean (SD) | 26.5 (2.4) |

BMI = Body mass index

Flushing

Among the 148 subjects that received all three treatments, flushing incidence was significantly higher after “R-Niacin ER Alone” (77%) than after “Concomitant ASA” (61%, p<0.001) or “ASA Pretreatment” (53%, p<0.001; Table 27).

TABLE 27
EFFECT OF ASPIRIN ON FLUSHING INCIDENCE

| | Treatment | | |
|---|---|---|---|
| | ASA | Concomitant | R-Niacin |
| | Pretreatment | ASA | ER Alone |
| N Subjects Dosed | 148 | 148 | 148 |
| N (%) Flushing | 79 (53%)*† | 91 (61%)* | 114 (77%) |
| N (%) Not Flushing | 69 (47%) | 57 (39%) | 34 (23%) |

*p < 0.001 versus R-Niacin ER Alone
†p = 0.090 versus Concomitant ASA

Neither aspirin-containing treatment was significantly different than the other in flushing incidence. As illustrated in FIG. 21, the incidence of the individual symptoms (redness, warmth, tingling, and itching) in the first overall flushing event was reduced by 30% to 50% after “ASA Pretreatment” compared with “R-Niacin ER Alone”. The least number of subjects reported all four symptoms after “ASA Pretreatment”, and by the most subjects after “R-Niacin ER Alone”. The individual symptoms were not compared between treatments. The number of flushing events followed the same trend as flushing incidence, with the highest number reported after “R-Niacin ER Alone” and the least number reported after “ASA Pretreatment” (data not shown).

In subjects who flushed after both “ASA Pretreatment” and “R-Niacin ER Alone” treatments (Table 28 below), “ASA Pretreatment” significantly decreased the intensity of first overall flushing events, measured either by categorical assessment or VAS (each p<0.001).

TABLE 28
EFFECT OF ASPIRIN PRETREATMENT ON THE FIRST OVERALL
FLUSHING EVENT

| | Treatment | | | |
|---|---|---|---|---|
| | ASA | R-Niacin ER | | |
| | Pretreatment | Alone | Difference* | P value^{†} |
| Incidence | | | | |
| N (%) Flushing | 71 (48%) | | — | — |
| after both | | | | |
| treatments | | | | |
| Intensity | | | | |
| (Categorical)^{‡} | | | | |
| N (%) Mild | 61 (86%) | 45 (63%) | 36% | <0.001 |
| N (%) Mod./ | 10 (14%) | 26 (37%) | −62% | |
| Severe^{§} | | | | |
| Intensity | | | | |
| (VAS, mm)^{‡} | | | | |
| Mean (SD) | 20.3 (15.2) | 30.8 (19.2) | −34.1% | <0.001 |
| Median | 18 | 33 | −45% | <0.001 |
| Min, Max | 0, 71 | 0, 90 | | |
| Duration (min)^{‡} | | | | |
| Mean (SD) | 82.7 (100.5) | 99.3 (91.1) | −16.7% | 0.171 |
| Median | 37 | 65 | −43% | 0.008 |
| Min, Max | 2, 393 | 5, 400 | | |

*Percent difference is relative to Niacin ER Alone.
^{†}From McNemar's test for incidence and intensity (categorical); for intensity (VAS) and duration, from paired t-test or Wilcoxon signed-rank test (mean or median data, respectively).
^{‡}Denominator is the number of subjects flushing after both treatments.
^{§}Moderate and severe categories were combined to allow 2 × 2 comparisons. No subject reported a severe event after ASA Pretreatment treatment; one subject reported a severe event after R-Niacin ER Alone treatment.

For both treatments, most of the flushing events were rated as mild, and only one (after “R-Niacin ER Alone”) was severe. The number of subjects with events rated as mild was 36% greater after “ASA Pretreatment” compared with “R-Niacin ER Alone”; correspondingly, the number of subjects with flushing rated as moderate or severe was 62% less. VAS ratings were more than 30% lower after “ASA Pretreatment” than after “R-Niacin ER Alone”. For duration of the first overall flushing event, the mean and median data were not consistent, suggesting non-normal distribution. Median duration for “ASA Pretreatment” was 43% less than for “R-Niacin ER Alone” (p=0.008). For the individual symptoms, redness, warmth, and tingling were significantly less intense after “ASA Pretreatment” (p<0.025, data not shown); there was no significant difference between treatments for the duration of any symptom.

In subjects who flushed after both “Concomitant ASA” and “R-Niacin ER Alone” treatments (Table 29 below), intensity of the first overall flushing event was significantly different for the categorical data (p=0.028), though not for the VAS data.

TABLE 29
EFFECT OF ASPIRIN COADMINISTRATION ON
THE FIRST OVERALL FLUSHING EVENT

| | Treatment | | | |
|---|---|---|---|---|
| | Concomitant | R-Niacin | | P |
| | ASA | ER Alone | Difference* | value^{†} |
| Incidence | | | | |
| N (%) Flushing | 80 (54%) | | — | — |
| after both | | | | |
| treatments | | | | |
| Intensity | | | | |
| (Categorical)^{‡} | | | | |
| N (%) Mild | 62 (78%) | 51 (64%) | 22% | 0.028 |
| N (%) Mod./ | 18 (23%) | 29 (36%) | −38% | |
| Severe^{§} | | | | |
| Intensity | | | | |
| (VAS, mm)^{‡} | | | | |
| Mean (SD) | 27.1 (19.4) | 31.0 (18.4) | −12.6% | 0.107 |
| Median | 23 | 33 | −30% | 0.213 |
| Min, Max | 0, 85 | 0, 90 | | |
| Duration | | | | |
| (min)^{‡} | | | | |
| Mean (SD) | 90.6 (109.6) | 100.6 (96.8) | −9.9% | 0.428 |
| Median | 43 | 68 | −37% | 0.354 |
| Min, Max | 3, 432 | 5, 400 | | |

*Percent difference is relative to Niacin ER Alone.
^{†}From McNemar's test for incidence and intensity (categorical); for intensity (VAS) and duration, from paired t-test or Wilcoxon signed-rank test (mean or median data, respectively).
^{‡}Denominator is the number of subjects flushing after both treatments.
^{§}Moderate and severe categories were combined to allow 2 × 2 comparisons. No subject reported a severe event after ASA Pretreatment treatment; one subject reported a severe event after R-Niacin ER Alone treatment.

Here, the number of subjects with mild flushing events after “Concomitant ASA” was 22% higher than after “R-Niacin ER Alone”, and the moderate or severe events were 38% less. The difference in duration of first overall flushing events was not significant. For the individual symptoms, the intensity of redness and warmth was significantly less after “Concomitant ASA” treatment (p<0.024, data not shown); there was no significant difference in the duration of any symptom.

In subjects who flushed after both “ASA Pretreatment” and “Concomitant ASA” treatments (see Table 30 below), differences in intensity of the first overall flushing events were not significant by categorical measure, but the 20% lower VAS scores for “ASA Pretreatment” was statistically significant.

TABLE 30
EFFECT OF ASPIRIN (BEFORE OR WITH REFORMULATED
NIACIN ER) ON THE FIRST OVERALL FLUSHING EVENT

| | Treatment | | | |
|---|---|---|---|---|
| | ASA | Concomitant | | |
| | Pretreatment | ASA | Difference* | P value^{†} |
| Incidence | | | | |
| N (%) Flushing | 60 (41%) | | — | — |
| after both | | | | |
| treatments | | | | |
| Intensity | | | | |
| (Categorical)^{‡} | | | | |
| N (%) Mild | 51 (85%) | 46 (76%) | 11% | 0.197 |
| N (%) Mod./ | 9 (15%) | 14 (24%) | −36% | |
| Severe^{§} | | | | |
| Intensity | | | | |
| (VAS, mm)^{‡} | | | | |
| Mean (SD) | 21.1 (15.3) | 27.1 (19.2) | −22.1% | 0.031 |
| Median | 19 | 23 | −17% | 0.048 |
| Min, Max | 0, 71 | 0, 85 | | |
| Duration | | | | |
| (min)^{‡} | | | | |
| Mean (SD) | 86.9 (105.6) | 99.1 (114.3) | −12.3% | 0.354 |
| Median | 35 | 48 | −27% | 0.226 |
| Min, Max | 2, 393 | 4, 432 | | |

*Percent difference is relative to Concomitant ASA.
^{†}From McNemar's test for incidence and intensity (categorical); for intensity (VAS) and duration, from paired t-test or Wilcoxon signed-rank test (mean or median data, respectively).
^{‡}Denominator is the number of subjects flushing after both treatments.
^{§}No severe events were reported for these treatments.

Duration of the first overall flushing events was not significantly different between these treatments. For the individual symptoms, neither intensity nor duration was significantly different between the two treatments.

The results above demonstrate that 650 mg (2×325 mg tablets) of aspirin taken 30 minutes before the extended-release tablets of the invention, significantly reduce the incidence, intensity, and duration of subject-reported flushing compared with the use of the tablets of the invention alone. Concomitant administration of aspirin 650 mg and the tablets of the invention reduced flushing incidence, intensity, and duration to a lesser extent.

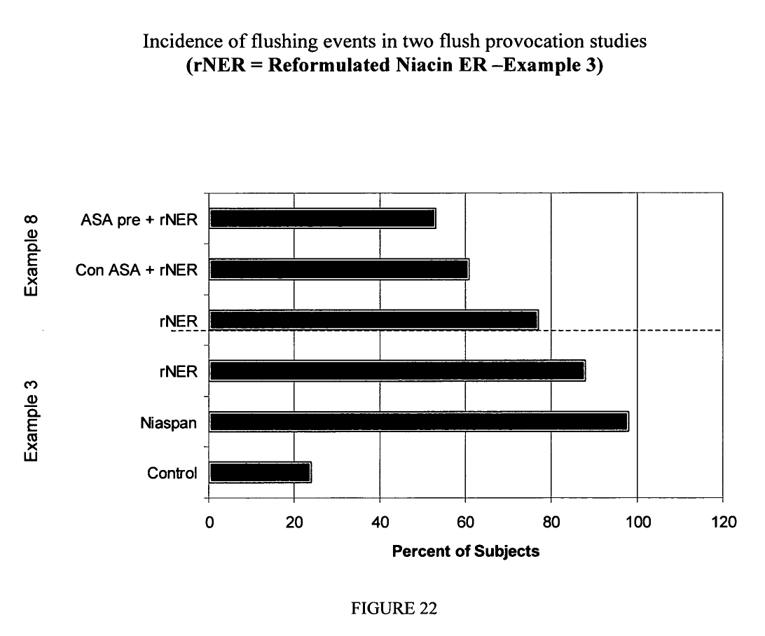

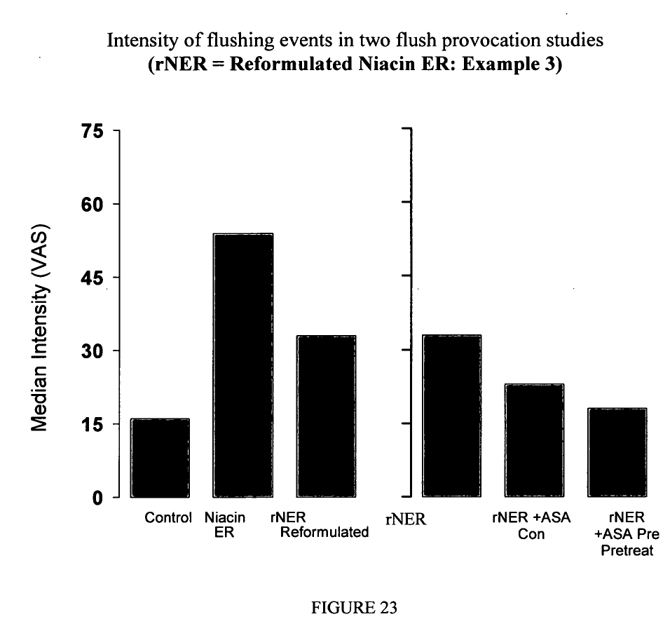

Flushing incidence and intensity results from Example 3 and Example 8 are summarized and illustrated together in FIGS. 22 and 23. These Figures show that the extended-release pharmaceutical compositions of the invention decrease flushing intensity and duration (˜40%) compared with the original 1000 mg tablet (Nisapan®)—see Example 3, although there is a small reduction in flushing incidence. Example 8 demonstrates that aspirin taken 30 minutes prior to or with the extended-release pharmaceutical compositions of the invention can reduce the incidence of flushing and further provide reductions in flushing intensity and duration. In Example 3, nearly all patients (98%) reported flushing (incidence) with the single 2000 mg dose of original 1000 mg tablet (2 tablet dose). In Example 8, only 50-60% of subjects flushed with a single 2000 mg dose of the extended-release tablets of the invention (2 tablet dose) plus aspirin. Median intensity with original 1000 mg tablet in the previous study was 54 mm on the VAS. In the current study, median intensity was only 19-23 mm with the extended-release tablets of the invention plus aspirin, and the vast majority (about 80% or more) reporting flushing to be ‘mild.’

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

We claim:

1. A 1000 mg niacin pharmaceutical composition comprising:
(a) about 78% to about 82% w/w of niacin;
(b) about 14% to about 18% w/w of hydroxypropyl methylcellulose having a methoxyl degree of substitution of about 1.39 to about 1.41 and a hydroxypropoxyl molar substitution of about 0.20 to about 0.22.;
(c) about 2.5% to about 3.0% w/w polyvinyl pyrrolidone, and
(d) about 0.95% to about 1.05% w/w stearic acid.

2. A pharmaceutical composition comprising:
(a) about 70% to about 92% w/w of niacin;
(b) about 7% to about 25% w/w of a release-retarding agent;
(c) about 0.1% to about 4.3% w/w of a binder, and
(d) about 0.5% to about 1.5% w/w of a lubricant;
wherein following administration to a patient, the composition results in reduced flushing compared to administration of a comparable dose of NIASPAN® tablets.

3. The pharmaceutical composition of claim 2 wherein said composition is a 1000 mg extended-release niacin tablet formulation.

4. The pharmaceutical composition of claim 3 wherein said composition is effective in reducing a serum lipid without causing treatment-limiting (i) hepatotoxicity and (ii) elevations in uric acid levels or glucose levels or both, following administration to said patient that would require such treatment to be discontinued when said composition is ingested by said patient once per day.

5. The pharmaceutical composition of claim 4 wherein administration to said patient is patient once per day during the evening or at night.

6. The pharmaceutical composition of claim 2 wherein the release-retarding agent is selected from the group consisting of hydroxypropyl cellulose (HPC), hydroxypropyl methyl cellulose (HPMC or hypromellose), methylcellulose (MC), hydroxyethyl cellulose (HEC), polyvinyl pyrrolidone (PVP) and xanthan gum, and a mixture thereof.

7. The pharmaceutical composition of claim 6 wherein the release-retarding agent is hydroxypropyl methylcellulose.

8. The pharmaceutical composition of claim 7 wherein the hydroxypropyl methylcellulose has a methoxyl degree of substitution of about 1.2 to about 2.0 and a hydroxypropoxyl molar substitution of about 0.1 to about 0.3.

9. The pharmaceutical composition of claim 8 wherein the hydroxypropyl methylcellulose has a methoxyl degree of substitution of about 1.4 to about 1.9 and a hydroxypropoxyl molar substitution of about 0.19 to about 0.24.

10. The pharmaceutical composition of claim 8 wherein the hydroxypropyl methylcellulose has a methoxyl degree of substitution of about 1.4 and a hydroxypropoxyl molar substitution of about 0.21.

11. The pharmaceutical composition of claim 8 wherein the hydroxypropyl methylcellulose has a viscosity of about 11,000 to about 22,000 mPas.

12. The pharmaceutical composition of claim 11 wherein the hydroxypropyl methylcellulose has a viscosity of about 13,000 to about 18,000 mPas.

13. The pharmaceutical composition of claim 2 further comprising a coating.

14. The pharmaceutical composition of claim 13 wherein said coating is a color coating having from about 1.5 to about 8.0% weight gain.

15. The pharmaceutical composition of claim 14 wherein said coating is a color coating applied to provide about 1.75 to about 5.0% weight gain to the tablet.

16. The pharmaceutical composition of 7 wherein said binder is selected from the group consisting of polyvinylpyrrolidone, hydroxypropyl cellulose, hydroxyethyl cellulose, ethylcellulose, polymethacrylate and waxes, or a mixture thereof.

17. The pharmaceutical composition of claim 16 wherein said binder is polyvinylpyrrolidone.

18. The pharmaceutical composition of claim 7 wherein said lubricant is selected from the group consisting of talc, magnesium stearate, calcium stearate, stearic acid and hydrogenated vegetable oils, and a mixture thereof.

19. The pharmaceutical composition of claim 18 wherein said lubricant is stearic acid.

20. The pharmaceutical composition of claim 2 comprising:
(a) about 76% to about 88% w/w of niacin;
(b) about 11.0% to about 20.0% w/w of a release-retarding agent;
(c) about 0.2% to about 3.25% w/w of a binder, and
(d) about 0.75% to about 1.25% w/w of a lubricant.

21. The pharmaceutical composition of claim 20 comprising:
(a) about 78% to about 82% w/w of niacin;
(b) about 14% to about 18% w/w of a release-retarding agent;
(c) about 2.5% to about 3.0% w/w of a binder, and
(d) about 0.85% to about 1.05% w/w of a lubricant.

22. The pharmaceutical composition of claim 21 comprising about 0.95% to about 1.05% w/w of a lubricant.

23. A method of reducing flushing associated with niacin treatment therapy, said method comprising administering a once daily pharmaceutical dosage form comprising
(a) about 70% to about 92% w/w of niacin;
(b) about 7% to about 25% w/w of a release-retarding agent;
(c) about 0.1% to about 4.3% w/w of a binder, and
(d) about 0.5% to about 1.5% w/w of a lubricant.

24. The method of claim 23 where said once daily dosage form comprises two 1000 mg tablets.

25. The method of claim 23 wherein said tablet is a 1000 mg tablet comprising
(a) about 76% to about 88% w/w of niacin;
(b) about 11.0% to about 20.0% w/w of a release-retarding agent;
(c) about 0.2% to about 3.25% w/w of a binder, and
(d) about 0.75% to about 1.25% w/w of a lubricant.

26. The method of claim 25 wherein said 1000 mg tablet comprises
(a) about 78% to about 82% w/w of niacin;
(b) about 14% to about 18% w/w of a release-retarding agent;
(c) about 2.5% to about 3.0% w/w of a binder, and
(d) about 0.85% to about 1.05% w/w of a lubricant.

27. The method of claim 26 wherein said 1000 mg tablet comprises about 0.95% to about 1.05% w/w of a lubricant.

28. The method of claim 23 wherein the release-retarding agent is selected from the group consisting of hydroxypropyl cellulose (HPC), hydroxypropyl methyl cellulose (HPMC or hypromellose), methylcellulose (MC), hydroxyethyl cellulose (HEC), polyvinyl pyrrolidone (PVP), methacrylate copolymers with trimethyl ammonioethylmethacrylate (EUDRAGIT RS®, EUDRAGIT RL®), and xanthan gum, and a mixture thereof.

29. The method of claim 28 wherein the release-retarding agent is hydroxypropyl methylcellulose and the hydroxypropyl methylcellulose has a methoxyl degree of substitution of about 1.2 to about 2.0 and a hydroxypropoxyl molar substitution of about 0.1 to about 0.3.

30. The method of claim 29 wherein the hydroxypropyl methylcellulose has a methoxyl degree of substitution of about 1.4 to about 1.9 and a hydroxypropoxyl molar substitution of about 0.19 to about 0.24.

31. The method of claim 30 wherein the hydroxypropyl methylcellulose is a has a methoxyl degree of substitution of about 1.4 and a hydroxypropoxyl molar substitution of about 0.21.

32. The method of claim 29 wherein the hydroxypropyl methylcellulose has a viscosity of about 11,000 to about 22,000 mPas.

33. The method of claim 32 wherein the hydroxypropyl methylcellulose has a viscosity of about 13,000 to about 18,000 mPas.

34. The method of claim 23 wherein the pharmaceutical dosage form further comprises a coating.

35. The method of claim 34 wherein said coating is a color coating applied to provide about 1.5 to about 8.0% weight gain to the pharmaceutical dosage form.

36. The method of claim 35 wherein said coating is a color coating applied to provide about 1.75 to about 5.0% weight gain to the pharmaceutical dosage form.

37. A method of preparing a direct compression niacin tablet comprising the steps of
(a) blending a mixture of about 70% to about 92% w/w of niacin, about 7% to about 25% w/w of a release-retarding agent, about 0.1% to about 4.3% w/w of a binder, and about 0.5% to about 1.5% w/w of a lubricant;
(b) compressing the mixture of step (a) into a tablet.

38. The method of claim 37 wherein said niacin tablet is a 1000 mg niacin dosage formulation.

39. The method of claim 37 further comprising coating the tablet.

40. The method of claim 37 further comprising coating the tablet with a color coating to provide about 1.5 to 8.0% weight gain to the tablet.

41. The method of claim 40 wherein said color coating has from about 1.75 to 5.0% weight gain.

42. The method of claim 40 wherein said release-retarding agent is selected from the group consisting of hydroxypropyl cellulose (HPC), hydroxypropyl methyl cellulose (HPMC or hypromellose), methylcellulose (MC), hydroxyethyl cellulose (HEC), polyvinyl pyrrolidone (PVP), methacrylate copolymers with trimethyl ammonioethylmethacrylate (EUDRAGIT RS®, EUDRAGIT RL®), and xanthan gum, or a mixture thereof.

43. The method of claim 40 wherein said binder is selected from the group consisting of polyvinylpyrrolidone, hydroxypropyl cellulose, hydroxyethyl cellulose, ethylcellulose, polymethacrylate and waxes, or a mixture thereof.

44. The method of claim 40 wherein said lubricant is selected from the group consisting of talc, magnesium stearate, calcium stearate, stearic acid and hydrogenated vegetable oils, or a mixture thereof.

45. The method of claim 40 wherein said tablet comprises about 76% to about 88% w/w of niacin, about 11.0% to about 20% w/w of a release-retarding agent, about 0.2% to about 3.25% w/w of a binder, and about 0.75% to about 1.25% w/w of a lubricant.

46. The method of claim 45 wherein said tablet comprises about 78% to about 82% w/w of niacin, about 14% to about 18% w/w of a release-retarding agent, about 2.5% to about 3.0% w/w of a binder, and about 0.95% to about 1.05% w/w of a lubricant.

47. The method of claim 37 wherein said release-retarding agent is hydroxypropyl methyl cellulose, said binder is polyvinylpyrrolidone, said lubricant is stearic acid and wherein hydroxypropyl methylcellulose has a methoxyl degree of substitution of about 1.2 to about 2.0 and a hydroxypropoxyl molar substitution of about 0.1 to about 0.3.

48. A direct compression 500 mg niacin extended-release tablet formulation comprising:
(a) about 65% to about 85% w/w of niacin;
(b) about 20% to about 32% w/w of a release-retarding agent;
(c) about 2% to about 3% w/w of a binder, and
(d) about 0.75% to about 1.25% w/w of a lubricant.

49. The direct compression 500 mg niacin extended-release tablet formulation of claim 48 comprising:
(a) about 68% to about 75% w/w of niacin;
(b) about 24% to about 29% w/w of a release-retarding agent;
(c) about 2.25% to about 2.75% w/w of a binder, and
(d) about 0.95% to about 1.05% w/w of a lubricant.

50. The direct compression 500 mg niacin extended-release tablet formulation of claim 48 further comprising a coating wherein said coating has from about 1.5 to about 8.0% weight gain.

51. A direct compression 750 mg niacin extended-release tablet formulation comprising:
(a) about 74% to about 80% w/w of niacin;
(b) about 16% to about 22% w/w of a release-retarding agent;
(c) about 2.5% to about 2.75% w/w of a binder, and
(d) about 0.75% to about 1.25% w/w of a lubricant.

52. The direct compression 750 mg niacin extended-release tablet formulation of claim 51 comprising:
(a) about 76% to about 79% w/w of niacin;
(b) about 18% to about 21% w/w of a release-retarding agent;
(c) about 2.5% to about 2.7% w/w of a binder, and
(d) about 0.95% to about 1.05% w/w of a lubricant.

53. The direct compression 750 mg niacin extended-release tablet formulation of claim 52 further comprising a coating wherein said coating has from about 1.5 to about 8.0% weight gain.

54. The pharmaceutical composition of claim 2 further comprising an anti-lipidemic agent.

55. The pharmaceutical composition of claim 54 wherein the anti-lipidemic agent is an HMG-CoA reductase inhibitor.

56. The pharmaceutical composition of claim 55 further comprising a flush-inhibiting agent.

57. The pharmaceutical composition of claim 2 further comprising a flush-inhibiting agent.

58. The pharmaceutical composition of claim 57 wherein the flush-inhibiting agent is a non-steroidal anti-inflammatory drug (NSAID).

59. The pharmaceutical composition of claim 58 wherein the flush-inhibiting agent is aspirin (ASA).

60. The pharmaceutical composition of claim 57 wherein the flush-inhibiting agent is a prostaglandin D2 receptor antagonist.

61. The pharmaceutical composition of claim 60 wherein the prostaglandin D2 receptor antagonist is MK-0524.

62. The method of any one of claims 23, 37, 48 or 51 wherein the niacin is granular niacin.

63. The method of claim 62 wherein the granular niacin particle size for is NLT 85% (w/w) for sieve fraction 100-425 μm and NMT 10% (w/w) for dust <100 μm.

64. A 1000 mg extended-release niacin pharmaceutical composition which when administered to a patient in need thereof as a single dose of two 1000 mg tablets, provides an in vivo plasma profile with a 90% CI for a natural-log transformed ratio within 80% to 125% for at least one of the following bioavailability parameters:
(a) NUA Cₘₐₓ of 2601.8 ng/mL;
(b) total recovery of urinary niacin 60.5%;
(c) niacin Cmax of 4958.9 ng/mL; and
(d) niacin AUC of 12414.5 ng/mL.

65. The 1000 mg extended-release niacin pharmaceutical composition of claim 64 wherein the natural-log transformed ratio is within 90% to 115%.

66. The 1000 mg extended-release niacin pharmaceutical composition of claim 64 wherein the natural-log transformed ratio is within 95% to 110%.

67. The 1000 mg extended-release niacin pharmaceutical composition of claim 64 further comprising at least on additional therapeutic agent selected from the group consisting of a flush-inhibiting agent and an anti-lipidemic agent.

68. The 1000 mg extended-release niacin pharmaceutical composition of claim 77 wherein said composition is effective in reducing a serum lipid without causing treatment-limiting (i) hepatotoxicity and (ii) elevations in uric acid levels or glucose levels or both, that would require such treatment to be discontinued when said composition is ingested by said patient once per day.

69. A 1000 mg extended-release niacin pharmaceutical composition which when administered to subjects in a bioequivalence study comparing a single dose of four 500 mg NIASPAN® tablets to a single dose of to of said 1000 mg extended-release niacin compositions provides 90% CI's for a natural-log transformed ratio of the appropriate bioavailability parameters within a 80% to 125% interval.

70. The 1000 mg extended-release niacin pharmaceutical composition of claim 69 wherein said bioavailability parameters are NUA Cₘₐₓ (ng/ml) and Total Recovery, or Niacin Cmax (ng/ml) and Niacin AUC.

71. A 1000 mg extended-release niacin pharmaceutical composition which when administered to a patient in need thereof as a single dose of two 1000 mg tablets, provides an in vivo plasma profile with a 90% CI for a natural-log transformed ratio within a 80% to 125% interval for at least one of the following bioavailability parameters:
(a) NUA Cₘₐₓ of about 2111.0 ng/mL to about 3253 ng/mL;
(b) total recovery of urinary niacin of about 49.24% to about 70.23%;
(c) niacin Cₘₐₓ of about 3096 ng/mL to about 6750 ng/mL; and
(d) niacin AUC of about 6723 ng/mL to about 18643 ng/mL.

72. The 1000 mg extended-release niacin pharmaceutical composition of claim 71 wherein said composition is effective in reducing a serum lipid without causing treatment-limiting (i) hepatotoxicity and (ii) elevations in uric acid levels or glucose levels or both, that would require such treatment to be discontinued when said composition is ingested by said patient once per day.

73. The pharmaceutical composition of claim 3, wherein niacin release is delayed.

74. The pharmaceutical composition of claim 73 further comprising an immediate-release flush inhibiting agent.

75. The pharmaceutical composition of claim 74 wherein the flush inhibiting agent is a prostaglandin D2 receptor.

76. The pharmaceutical composition of claim 75 wherein the prostaglandin D2 receptor is MK-0524.

77. The pharmaceutical composition of claim 74 wherein the flush inhibiting agent is a non-steroidal anti-inflammatory drug (NSAID).
